(19) United States
(12) Patent Application Publication
(10) Pub. No.: US 2025/0213708 A1
(43) Pub. Date: Jul. 3, 2025

(54) CHIMERIC TRANSMEMBRANE PROTEINS AND USES THEREOF

(71) Applicant: KITE PHARMA, INC., Santa Monica, CA (US)

(72) Inventors: Peter EMTAGE, Lafayette, CA (US); Rosa VINCENT, New York, NY (US)

(21) Appl. No.: 19/082,896

(22) Filed: Mar. 18, 2025

Related U.S. Application Data
(63) Continuation of application No. 17/040,009, filed on Sep. 21, 2020, now Pat. No. 12,280,119.
(60) Provisional application No. 62/647,498, filed on Mar. 23, 2018.

(51) Int. Cl. A61K 47/65; A61K 40/11; A61K 40/31; A61K 40/42; C07K 14/55; C07K 14/705; C07K 14/715; C07K 14/725

(52) U.S. Cl. CPC A61K 47/65; A61K 40/11; A61K 40/31; A61K 40/4211; C07K 14/55; C07K 14/7051; C07K 14/70578; C07K 14/7155

(57) ABSTRACT

Provided herein are chimeric transmembrane proteins, nucleic acids encoding these chimeric transmembrane proteins, and mammalian cells containing these nucleic acids, and methods of making and using these mammalian cells.

### CROSS-REFERENCE TO RELATED APPLICATIONS

This application is a continuation of U.S. application Ser. No. 17/040,009, filed Sep. 21, 2020, the entire contents of which are hereby incorporated herein by reference. U.S. application Ser. No. 17/040,009, is a U.S. national stage of International Application No. PCT/US2019/023424, filed Mar. 21, 2019, and claims benefit of U.S. Provisional Patent Application Ser. No. 62/647,498, filed on Mar. 23, 2018, the entire contents of which are herein incorporated by reference.

### REFERENCE TO SEQUENCE LISTING

The Sequence Listing submitted herewith as an .xml file named “82352-422057_SeqListing” created on Mar. 17, 2025, and having a size of 102,300 bytes is hereby incorporated by reference pursuant to 37 C.F.R. § 1.52 (c) (5).

### TECHNICAL FIELD

The present disclosure relates to the field of biotechnology, and more specifically, to single-chain and multi-chain chimeric antigen receptors.

### BACKGROUND

Interleukin-2 receptor is expressed on the cell surface (either temporary or permanent) in almost all hematopoietic cells including lymphoid linages T, B, and NK cells, as well as myeloid ones like macrophages, monocytes, and neutrophils. Upon binding to IL-2, the interleukin-2 receptor transduces a signal into the cell via activation of the Janus kinases—JAK1 and JAK3. The phosphorylation of the cytoplasmic domain of the ϑ-chain of the interleukin-2 receptor enables homodimer formation of STAT-3 and STAT-5 factors. Homodimers of STAT-3 and STAT-5 migrate to the nucleus and enhance transcription of IL-2-dependent genes (Zlatko Dembic, Chapter 6, Cytokines of the Immune System, Academic Press, 2015, pages 143-239).

While many improvements have been made to generate a secondary proliferative signal in second generation chimeric antigen receptor (CAR) T-cells (through the inclusion of co-stimulatory signaling domains such as CD28 and 4-1 BB in chimeric antigen receptors), this signal remains antigen-dependent often does not have sufficient co-stimulatory activity in solid tumors that have low tumor associated antigen levels.

In another attempt to provide sufficient co-stimulatory activity in CAR T-cells, co-administration of cytokines belonging to the common gamma (γc) chain receptor family have been used. However, prolonged usage of recombinant, pro-proliferative cytokines, such as IL-2, is often associated with severe side effects, limiting the duration and dosage of administration. In view of the above, alternative, improved methods of providing a co-stimulatory signal to CAR T-cells are desired.

### SUMMARY

The present disclosure is based, at least in part, on the discovery that a chimeric transmembrane protein including an extracellular IL-2 domain, an extracellular sushi domain of an alpha chain of an interleukin-2 receptor, and a transmembrane domain of an alpha chain of an interleukin-2 receptor provide for antigen-independent co-stimulation of a chimeric antigen receptor (CAR) T-cell. In view of this discovery, provided herein are chimeric antigen receptors that include an extracellular IL-2 domain, an extracellular sushi domain from an alpha chain of interleukin-2 receptor, and a transmembrane domain of an alpha chain of interleukin-2 receptor. Also provided are nucleic acids encoding these chimeric transmembrane proteins, vectors including any of these nucleic acids, and mammalian cells that include any of these nucleic acids or vectors. Also provided herein are methods of treating a cancer, methods of inducing cell death in a cancer cell (e.g., apoptosis and/or necrosis), and methods of decreasing the risk of developing a metastasis or an additional metastasis in a subject in need thereof that include administering any of the mammalian cells described herein to the subject.

Provided herein are chimeric transmembrane proteins that include: an extracellular IL-2 domain; an extracellular sushi domain from an alpha chain of interleukin-2 receptor; and a transmembrane domain of an alpha chain of interleukin-2 receptor. In some embodiments of any of the chimeric transmembrane proteins described herein, the extracellular IL-2 domain includes a sequence of a wildtype IL-2 protein. In some embodiments of any of the chimeric transmembrane proteins described herein, the wildtype IL-2 protein is a wildtype human IL-2 protein. In some embodiments of any of the chimeric transmembrane proteins described herein, the wildtype human IL-2 protein includes a sequence of SEQ ID NO: 1. In some embodiments of any of the chimeric transmembrane proteins described herein, the extracellular IL-2 domain includes a sequence that is at least 80% identical to the sequence of a wildtype IL-2 protein. In some embodiments of any of the chimeric transmembrane proteins described herein, the extracellular IL-2 domain includes a sequence that is at least 95% identical to the sequence of a wildtype IL-2 protein. In some embodiments of any of the chimeric transmembrane proteins described herein, the wildtype IL-2 protein is a wildtype human IL-2 protein. In some embodiments of any of the chimeric transmembrane proteins described herein, the wildtype human IL-2 protein is SEQ ID NO: 1. In some embodiments of any of the chimeric transmembrane proteins described herein, the extracellular IL-2 domain is a sequence of a wildtype IL-2 protein having one or both of (i) one to ten amino acids removed from the N-terminus of the sequence of the wildtype IL-2 protein, and (ii) one to ten amino acids removed from the C-terminus of the sequence of the wildtype IL-2 protein. In some embodiments of any of the chimeric transmembrane proteins described herein, the wildtype IL-2 protein is a human wildtype IL-2 protein. In some embodiments of any of the chimeric transmembrane proteins described herein, the human wildtype IL-2 protein is SEQ ID NO: 1.

In some embodiments of any of the chimeric transmembrane proteins described herein, the chimeric transmembrane protein further includes a linker sequence positioned between the extracellular IL-2 domain and the extracellular sushi domain. In some embodiments of any of the chimeric transmembrane proteins described herein, the linker sequence is 2 amino acids to about 50 amino acids (e.g., 4 amino acids to about 40 amino acids). In some embodiments of any of the chimeric transmembrane proteins described herein, the linker sequence is a naturally-occurring amino acid sequence. In some embodiments of any of the chimeric transmembrane proteins described herein, the linker sequence is not a naturally-occurring amino acid sequence. In some embodiments of any of the chimeric transmembrane proteins described herein, the linker sequence includes a sequence of SEQ ID NO: 2. In some embodiments of any of the chimeric transmembrane proteins described herein, the linker sequence consists of a sequence of SEQ ID NO: 2.

Some embodiments of any of the chimeric transmembrane proteins described herein further include an additional linker sequence positioned between the extracellular sushi domain and the transmembrane domain. In some embodiments of any of the chimeric transmembrane proteins described herein, the additional linker sequence is 2 amino acids to about 50 amino acids (e.g., 4 amino acids to about 40 amino acids). In some embodiments of any of the chimeric transmembrane proteins described herein, the additional linker sequence is a naturally-occurring amino acid sequence. In some embodiments of any of the chimeric transmembrane proteins described herein, the additional linker sequence is not a naturally-occurring amino acid sequence. In some embodiments of any of the chimeric transmembrane proteins described herein, the linker sequence includes a sequence of SEQ ID NO: 2. In some embodiments of any of the chimeric transmembrane proteins described herein, the linker sequence consists of a sequence of SEQ ID NO: 2.

In some embodiments of any of the chimeric transmembrane proteins described herein, the extracellular sushi domain includes a sushi domain from a wildtype alpha chain of interleukin-2 receptor. In some embodiments of any of the chimeric transmembrane proteins described herein, the wildtype alpha chain of interleukin-2 receptor is wildtype human alpha chain of interleukin-2 receptor. In some embodiments of any of the chimeric transmembrane proteins described herein, the extracellular sushi domain of the wildtype human alpha chain of interleukin-2 receptor includes one or both of the sequence of SEQ ID NO: 3 and SEQ ID NO: 4. In some embodiments of any of the chimeric transmembrane proteins described herein, the extracellular sushi domain is at least 80% identical to a sequence of an extracellular sushi domain of a wildtype alpha chain of interleukin-2 receptor. In some embodiments of any of the chimeric transmembrane proteins described herein, the extracellular sushi domain is at least 95% identical to the sequence of the extracellular sushi domain of a wildtype alpha chain of interleukin-2 receptor. In some embodiments of any of the chimeric transmembrane proteins described herein, the wildtype alpha chain of interleukin-2 receptor is a wildtype human alpha chain of interleukin-2 receptor. In some embodiments of any of the chimeric transmembrane proteins described herein, the extracellular sushi domain of the wildtype human alpha chain of interleukin-2 receptor includes one or both of the sequences of SEQ ID NO: 3 and SEQ ID NO: 4. In some embodiments of any of the chimeric transmembrane proteins described herein, the extracellular sushi domain is a sequence of an extracellular sushi domain of a wildtype alpha chain of interleukin-2 receptor having one or both of (i) one to five amino acids removed from the N-terminus of the sequence of the extracellular sushi domain of the wildtype alpha chain of interleukin-2 receptor, and (ii) one to five amino acids removed from the C-terminus of the sequence of the extracellular sushi domain of the wildtype alpha chain of interleukin-2 receptor. In some embodiments of any of the chimeric transmembrane proteins described herein, the wildtype alpha chain of interleukin-2 receptor is a wildtype human alpha chain of interleukin-2 receptor. In some embodiments of any of the chimeric transmembrane proteins described herein, the sequence of the extracellular sushi domain of the wildtype human alpha chain of interleukin-2 receptor includes one or both of the sequence of SEQ ID NO: 3 or SEQ ID NO: 4.

In some embodiments of any of the chimeric transmembrane proteins described herein, the transmembrane domain includes a transmembrane domain from a wildtype alpha chain of interleukin-2 receptor. In some embodiments of any of the chimeric transmembrane proteins described herein, the wildtype alpha chain of interleukin-2 receptor is wildtype human alpha chain of interleukin-2 receptor. In some embodiments of any of the chimeric transmembrane proteins described herein, the transmembrane domain of the wildtype human alpha chain of interleukin-2 receptor includes a sequence of SEQ ID NO: 5. In some embodiments of any of the chimeric transmembrane proteins described herein, the transmembrane domain is at least 80% identical to a sequence of a transmembrane domain of a wildtype alpha chain of interleukin-2 receptor. In some embodiments of any of the chimeric transmembrane proteins described herein, the transmembrane domain is at least 95% identical to the sequence of the transmembrane domain of a wildtype alpha chain of interleukin-2 receptor. In some embodiments of any of the chimeric transmembrane proteins described herein, the wildtype alpha chain of interleukin-2 receptor is a wildtype human alpha chain of interleukin-2 receptor. In some embodiments of any of the chimeric transmembrane proteins described herein, the transmembrane domain of the wildtype human alpha chain of interleukin-2 receptor is SEQ ID NO: 5. In some embodiments of any of the chimeric transmembrane proteins described herein, the transmembrane domain is a sequence of a transmembrane domain of a wildtype alpha chain of interleukin-2 receptor having one or both of (i) one to ten amino acids removed from the N-terminus of the sequence of the transmembrane domain of the wildtype alpha chain of interleukin-2 receptor, and (ii) one to ten amino acids removed from the C-terminus of the sequence of the transmembrane domain of the wildtype alpha chain of interleukin-2 receptor. In some embodiments of any of the chimeric transmembrane proteins described herein, the wildtype alpha chain of interleukin-2 receptor is a wildtype human alpha chain of interleukin-2 receptor. In some embodiments of any of the chimeric transmembrane proteins described herein, the sequence of the transmembrane domain of the wildtype human alpha chain of interleukin-2 receptor is SEQ ID NO: 5.

In some embodiments of any of the chimeric transmembrane proteins described herein, the chimeric transmembrane protein further includes an intracellular domain of an alpha chain of interleukin-2 receptor. In some embodiments of any of the chimeric transmembrane proteins described herein, the intracellular domain includes a sequence of an intracellular domain of a wildtype alpha chain of interleukin-2 receptor. In some embodiments of any of the chimeric transmembrane proteins described herein, the wildtype alpha chain of interleukin-2 receptor is a wildtype human alpha chain of interleukin-2 receptor. In some embodiments of any of the chimeric transmembrane proteins described herein, the intracellular domain of the wildtype alpha chain of interleukin-2 receptor includes a sequence of SEQ ID NO: 6. In some embodiments of any of the chimeric transmembrane proteins described herein, the intracellular domain includes a sequence that is at least 80% identical to a sequence of an intracellular domain of a wildtype alpha chain of interleukin-2 receptor. In some embodiments of any of the chimeric transmembrane proteins described herein, the intracellular domain includes a sequence that is at least 95% identical to the sequence of the intracellular domain of the wildtype alpha chain of interleukin-2 receptor. In some embodiments of any of the chimeric transmembrane proteins described herein, the wildtype alpha chain of interleukin-2 receptor is a wildtype human alpha chain of interleukin-2 receptor. In some embodiments of any of the chimeric transmembrane proteins described herein, the sequence of the intracellular domain of the wildtype human alpha chain of interleukin-2 receptor is SEQ ID NO: 6. In some embodiments of any of the chimeric transmembrane proteins described herein, the intracellular domain is a sequence of an intracellular domain of a wildtype alpha chain of interleukin-2 receptor having one or both of (i) one to ten amino acids removed from the N-terminus of the sequence of the intracellular domain of the wildtype alpha chain of interleukin-2 receptor, and (ii) one to ten amino acids removed from the C-terminus of the sequence of the intracellular domain of the wildtype alpha chain of interleukin-2 receptor. In some embodiments of any of the chimeric transmembrane proteins described herein, the wildtype alpha chain of interleukin-2 receptor is a wildtype human alpha chain of interleukin-2 receptor. In some embodiments of any of the chimeric transmembrane proteins described herein, the sequence of the intracellular domain of the wildtype human alpha chain of interleukin-2 receptor is SEQ ID NO: 6.

Also provided are nucleic acids encoding any of the chimeric transmembrane proteins described herein. Also provided are vectors that include any of the nucleic acids encoding any of the chimeric transmembrane proteins described herein. Some embodiments of any of the vectors described herein, further include a promoter sequence operably linked to the nucleic acid encoding any of the chimeric transmembrane proteins described herein, and optionally, an enhancer sequence operably linked to the nucleic acid encoding any of the chimeric transmembrane proteins described herein. Some embodiments of any of the vectors described herein further include a poly(A) sequence operably linked to the nucleic acid encoding any of the chimeric transmembrane proteins described herein. Some embodiments of any of the vectors described herein further include a sequence encoding a chimeric antigen receptor. In some embodiments of any of the vectors described herein, the chimeric antigen receptor binds specifically to a tumor antigen. In some embodiments of any of the vectors described herein, the tumor antigen is selected from the group of: glypican-3, BCMA, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, IL-13, IL-6, insulin-like growth factor I receptor, integrin a51, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. In some embodiments of any of the vectors described herein, the tumor antigen is glypican-3. In includes one or more co-stimulatory signaling domains selected from the group consisting of: 4-1BB, CD27, OX40, CD40, CD28, GITR, CD2, CD5, ICAM-1, CD1la, Lck, TNFR-I, TNFR-II, FasR, CD30, ICOS, LIGHT, NKG2C, B7-H3, DAP-10, and DAP-12. In some embodiments of any of the vectors described herein, the vector is a lentiviral or adenoviral vector.

Also provided herein are mammalian cells that include any of the nucleic acids encoding any of the chimeric transmembrane proteins described herein or any of the vectors described herein. In some embodiments of any of the mammalian cells described herein, the mammalian cell is an immune cell. In some embodiments of any of the mammalian cells described herein, the immune cell is selected from the group of: a CD4+ T cell, a CD8+ T cell, a B cell, a monocyte, a natural killer cell, a dendritic cell, a macrophage, a regulatory T cell, and a helper T cell. In some embodiments of any of the mammalian cells described herein, the mammalian cell was previously obtained from a subject or is a daughter cell of a mammalian cell that was previously obtained from the subject. In some embodiments of any of the mammalian cells described herein, the mammalian cell is a human cell.

Also provided herein are pharmaceutical compositions that include any of the vectors described herein and a pharmaceutically acceptable carrier. Also provided herein are pharmaceutical compositions that include any of the mammalian cells described herein.

Also provided herein are sets of vectors that include: a first vector that is any of the vectors described herein that include any of the nucleic acids encoding any of the chimeric transmembrane proteins described herein; and a second vector that includes a sequence encoding a chimeric antigen receptor. In some embodiments of any of the sets of vectors described herein, the chimeric antigen receptor binds specifically to a tumor antigen. In some embodiments of any of the sets of vectors described herein, the tumor antigen is selected from the group of: glypican-3, BCMA, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1receptor, IGF-I, IgG1, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-RI, TRAIL-R2, tumor antigen CTAA16.88, VEGF-A, VEGFR-1, VEGFR2, and vimentin. In some embodiments of any of the sets of vectors described herein, the tumor antigen is glypican-3. In some embodiments of any of the sets of vectors described herein, the chimeric antigen receptor includes one or more co-stimulatory signaling domains selected from the group consisting of: 4-1BB, CD27, OX40, CD40, CD28, GITR, CD2, CD5, ICAM-1, CD11a, Lck, TNFR-I, TNFR-II, FasR, CD30, ICOS, LIGHT, NKG2C, B7-H3, DAP-10, and DAP-12. In some embodiments of any of the sets of vectors described herein, one or both of the first vector and the second vector is a lentiviral or adenoviral vector. In some embodiments of any of the sets of vectors described herein, the second vector further includes a promoter operably linked to the sequence encoding the chimeric antigen receptor, and optionally, an enhancer operably linked to the sequence encoding the chimeric antigen receptor. In some embodiments of any of the sets of vectors described herein, the second vector further includes a poly(A) sequence operably linked to the sequence encoding the chimeric antigen receptor.

Also provided herein are mammalian cells that include any of the sets of vectors described herein. In some embodiments of any of the mammalian cells described herein, the mammalian cell is an immune cell. In some embodiments of any of the mammalian cells described herein, the immune cell is selected from the group of: a CD4+ T cell, a CD8+ T cell, a B cell, a monocyte, a natural killer cell, a dendritic cell, a macrophage, a regulatory T cell, and a helper T cell. In some embodiments of any of the mammalian cells described herein, the mammalian cell was previously obtained from a subject or is a daughter cell of a mammalian cell that was previously obtained from the subject. In some embodiments of any of the mammalian cells described herein, the mammalian cell is a human cell.

Also provided herein are pharmaceutical compositions that include any of the sets of vectors described herein and a pharmaceutically acceptable carrier. Also provided herein are pharmaceutical compositions that include a mammalian cell including any of the sets of vectors described herein. Also provided herein are kits that include any of the pharmaceutical compositions described herein.

Also provided herein are methods of treating a cancer in a subject in need thereof that include administering a therapeutically effective amount of any of the mammalian cells described herein to the subject. In some embodiments of any of the methods described herein, the subject is a human, and the mammalian cell is human. In some embodiments of any of the methods described herein, the mammalian cell was previously obtained from the subject or is a daughter cell of a cell previously obtained from the subject.

Also provided herein are methods of reducing the volume of a solid tumor in a subject in need thereof that include administering a therapeutically effective amount of any of the mammalian cells described herein to the subject. In some embodiments of any of the methods described herein, the subject is a human, and the mammalian cell is human. In some embodiments of any of the methods described herein, the mammalian cell was previously obtained from the subject or is a daughter cell of a cell previously obtained from the subject.

Also provided herein are methods of inducing cell death in a cancer cell in a subject in need thereof that include administering a therapeutically effective amount of any of the mammalian cells described herein to the subject. In some embodiments of any of the methods described herein, the subject is a human, and the mammalian cell is human. In some embodiments of any of the methods described herein, the mammalian cell was previously obtained from the subject or is a daughter cell of a cell previously obtained from the subject.

Also provided herein are methods of decreasing the risk of developing a metastasis or an additional metastasis in a subject having a cancer that include administering a therapeutically effective amount of any of the mammalian cells described herein to the subject. In some embodiments of any of the methods described herein, the subject is a human, and the mammalian cell is human. In some embodiments of any of the methods described herein, the mammalian cell was previously obtained from the subject or is a daughter cell of a cell previously obtained from the subject.

The use of the term “a” before a noun is meant “one or more” of the particular noun. For example, the phrase “a mammalian cell” means “one or more mammalian cell.”

The terms “chimeric antigen receptor” and “CAR” are used interchangeably herein, and refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. CAR molecules and derivatives thereof (e.g., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2): 141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. A CAR can be a single-chain chimeric antigen receptor or a multi-chain chimeric antigen receptor.

The term “transmembrane domain” means a domain of a polypeptide that includes at least one contiguous amino acid sequence that traverses a lipid bilayer when present in the corresponding endogenous polypeptide when expressed in a mammalian cell. For example, a transmembrane domain can include one, two, three, four, five, six, seven, eight, nine, or ten contiguous amino acid sequences that each traverse a lipid bilayer when present in the corresponding endogenous polypeptide when expressed in a mammalian cell. As is known in the art, a transmembrane domain can, e.g., include at least one (e.g., two, three, four, five, six, seven, eight, nine, or ten) contiguous amino acid sequence (that traverses a lipid bilayer when present in the corresponding endogenous polypeptide when expressed in a mammalian cell) that has α-helical secondary structure in the lipid bilayer. In some embodiments, a transmembrane domain can include two or more contiguous amino acid sequences (that each traverse a lipid bilayer when present in the corresponding endogenous polypeptide when expressed in a mammalian cell) that form a B-barrel secondary structure in the lipid bilayer. Non-limiting examples of transmembrane domains are described herein. Additional examples of transmembrane domains are known in the art.

The term “antigen-binding domain” means a domain that binds specifically to a target antigen. In some examples, an antigen-binding domain can be formed from the amino acids present within a single-chain polypeptide. In other examples, an antigen-binding domain can be formed from amino acids present within a first single-chain polypeptide and the amino acids present in one or more additional single-chain polypeptides (e.g., a second single-chain polypeptide). Non-limiting examples of antigen-binding domains are described herein, including, without limitation, scFvs, or LBDs of growth factors. Additional examples of antigen-binding domains are known in the art.

As used herein, the term “antigen” refers generally to a binding partner specifically recognized by an antigen-binding domain described herein. Exemplary antigens include different classes of molecules, such as, but not limited to, polypeptides and peptide fragments thereof, small molecules, lipids, carbohydrates, and nucleic acids. Non-limiting examples of antigen or antigens that can be specifically bound by any of the antigen-binding domains are described herein. Additional examples of antigen or antigens that can be specifically bound by any of the antigen-binding domains are known in the art.

The term “intracellular signaling domain” means an intracellular signaling domain from an endogenous signaling transmembrane polypeptide expressed in an immune cell (e.g., a T lymphocyte) that promotes downstream immune cell signaling (e.g., T-cell receptor signaling) and/or immune cell activation (e.g., T cell activation). Non-limiting examples of intracellular signaling domains are described herein. Additional examples of intracellular signaling domains are known in the art. See, e.g., Chen et al., Nature Reviews Immunol. 13:227-242, 2013.

The term “immunoreceptor tyrosine-based activation motif” or “ITAM)” means an amino acid motif that includes a four amino-acid consensus sequence of a tyrosine separated from a leucine or an isoleucine by two other amino acids (YxxL/I). The tyrosine residue in the four-amino acid consensus sequence becomes phosphorylated following interaction of a signaling pathway kinase (e.g., a lymphocyte signaling pathway kinase). Non-limiting examples of ITAMs are described herein. Additional examples of ITAMs are known in the art.

The phrase “treatment of cancer” means a reduction in the number, frequency, or severity of one or more (e.g., two, three, four, or five) symptoms of a cancer in a subject having a cancer.

As used herein, “extracellular domain” describes a portion of a polypeptide (e.g., a domain) that is present in the extracellular space when the polypeptide is expressed in a mammalian cell (e.g., a human cell).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 is a schematic drawing of an exemplary chimeric transmembrane protein comprising an extracellular interleukin-2 (IL-2) domain, an extracellular sushi domain from the alpha chain of interleukin-2 receptor (IL-2Rα), a transmembrane domain of an alpha chain of interleukin-2 receptor, and a linker sequence positioned between the extracellular IL-2 domain and the extracellular sushi domain.

FIG. 2 is a schematic drawing of a vector encoding an exemplary chimeric antigen receptor used to transduce a T cell. The vector contains a SFFV promoter; a sequence encoding an exemplary chimeric antigen receptor comprising a FMC63 antigen-binding domain (anti-CD19), a 4-1BB costimulatory signaling domain, and a CD3ζ ITAM; and a sequence encoding an exemplary chimeric transmembrane shown in FIG. 1.

FIG. 3 shows results from flow cytometry experiments using primary human T cells transduced with the vector as shown in FIG. 2. An antibody against His-tag (APC-A::HIS) was used to detect the His-tagged chimeric transmembrane protein of SEQ ID NO: 7. An antibody against c-Myc (PE-A::Myc) was used to detect the chimeric antigen receptor (FMC63-41BB-CD3z CAR). Upon transduction of the vector, primary human T cells expressing both the CAR molecule and the chimeric transmembrane protein were detected.

FIG. 4 shows results from flow cytometry experiments using primary human T cells untransduced, or transduced with the chimeric transmembrane protein of SEQ ID NO: 7 alone (“mbIL2”), the chimeric antigen receptor including a FMC63 antigen-binding domain, a 4-1BB costimulatory signaling domain, and a CD3 ITAM alone (FMC63-41BB-CD3z CAR)(“FMC63”), or the vector shown in FIG. 2 encoding both FMC63-41BB-CD3z CAR and the chimeric transmembrane protein of SEQ ID NO: 7 (“mbIL2-FMC63”). The antibodies used include an antibody against CD4 (Comp-BV785-A::CD4), an antibody against CD8 (Comp-PE-A::CD8), an antibody against CD45RO (Comp-APC-A::C45RO), and an antibody against CD62L (Comp-BV605-A::CD62L). An asterisk indicates that the cells were cultured in absence of soluble IL-2.

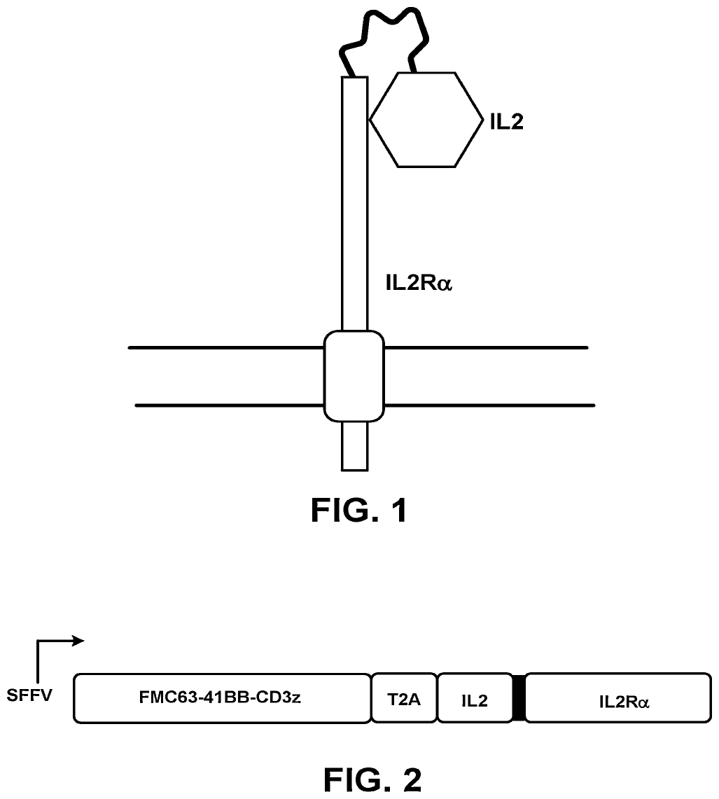

### DETAILED DESCRIPTION

Provided herein are chimeric antigen receptors that include an extracellular IL-2 domain, an extracellular sushi domain from an alpha chain of interleukin-2 receptor, and a transmembrane domain of an alpha chain of interleukin-2 receptor. Also provided are nucleic acids encoding these chimeric transmembrane proteins, vectors including any of these nucleic acids, and mammalian cells that include any of these nucleic acids or vectors. Also provided herein are methods of treating a cancer, methods of inducing cell death in a cancer cell (e.g., apoptosis and/or necrosis), and methods of decreasing the risk of developing a metastasis or an additional metastasis in a subject in need thereof that include administering any of the mammalian cells described herein to the subject.

The chimeric transmembrane proteins provided herein can be used to maintain CAR T-cells in the absence of exogenous cytokine support or antigen stimulation, thus providing for the T-cell stimulatory effects of IL-2 without the dose-limiting toxicities associated with prolonged administration of soluble, recombinant IL-2.

Non-limiting aspects of the chimeric transmembrane proteins, nucleic acids, vectors, mammalian cells, and methods provided herein are described below, and can be used in any combination without limitation. Additional aspects of these chimeric transmembrane proteins, nucleic acids, vectors, mammalian cells, and methods are known in the art.

### Chimeric Transmembrane Proteins

Provided herein are chimeric transmembrane proteins that include an extracellular IL-2 domain (e.g., any of the exemplary extracellular IL-2 domains described herein or known in the art), an extracellular sushi domain from an alpha chain of interleukin-2 receptor (e.g., one or more of any of the exemplary extracellular sushi domains from an alpha chain of interleukin-2 receptor described herein or known in the art), and a transmembrane domain of an alpha chain of interleukin-2 receptor (e.g., any of the exemplary transmembrane domains of an alpha chain of interleukin-2 receptor described herein or known in the art). In some embodiments, the chimeric transmembrane protein comprises a linker sequence positioned between the extracecullar IL-2 domain and the extracecullar sushi domain (e.g., any of the exemplary linker sequences described herein or known in the art). In some embodiments, the chimeric transmembrane protein further comprises an additional linker positioned between the extracecullar sushi domain and the transmembrane domain of the alpha chain of the interleukin-2 receptor (e.g., any of the exemplary linker sequences described herein or known in the art). In some embodiments, the chimeric transmembrane protein can further include an intracellular domain of an alpha chain of interleukin-2 receptor (e.g., any of the exemplary intracellular domains of an alpha chain of interleukin-2 receptor described herein or known in the art).

In some embodiments, the chimeric transmembrane protein comprises or is SEQ ID NO: 7 (shown below).

Exemplary Chimeric Transmembrane Protein
(SEQ ID NO: 7)
MLLLVTSLLLCELPHPAFLLIPHHHHHHAPTSSSTKKTQLQLEHLLLDLQ
MILNGINNYKNPKLTRMLTFKFYMPKKATELKHLQCLEEELKPLEEVLNL
AQSKNFHLRPRDLISNINVIVLELKGSETTFMCEYADETATIVEFLNRWI
TFCQSllSTLTSGGGSGGGGSGGGGSGGGGSGGGSLQELCDDDPPEIPHA
TFKAMAYKEGTMLNCECKRGFRRIKSGSLYMLCTGNSSHSSWDNQCQCTS
SATRNTTKQVTPQPEEQKERKTTEMQSPMQPVDQASLPGHCREPPPWENE
ATERIYHFVVGQMVYYQCVQGYRALHRGPAESVCKMTHGKTRWTQPQLIC
TGEMETSQFPGEEKPQASPEGRPESETSCLVTTTDFQIQTEMAATMETSI
FTTEYQVAVAGCVFLLISVLLLSGLTWQRRQRKSRRTI

In some embodiments, the chimeric transmembrane protein comprises or is a sequence that is at least 60% identical, at least 65% identical, at least 70% identical, at least 75% identical, at least 80% identical, at least 82% identical, at least 84% identical, at least 86% identical, at least 88% identical, at least 90% identical, at least 91% identical, at least 92% identical, at least 94% identical, at least 95% identical, at least 96% identical, at least 97% identical, at least 98% identical, at least 99% identical, or 100% identical to SEQ ID NO: 7.

In some embodiments, the chimeric transmembrane protein comprises or is SEQ ID NO: 8 (shown below).

Exemplary Chimeric Transmembrane Protein
(SEQ ID NO: 8)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSllSTLTSGGGSGGGGSGGGGSGG
GGSGGGSLQELCDDDPPEIPHATFKAMAYKEGTMLNCECKRGFRRIKSGS
LYMLCTGNSSHSSWDNQCQCTSSATRNTTKQVTPQPEEQKERKTTEMQSP
MQPVDQASLPGHCREPPPWENEATERIYHFVVGQMVYYQCVQGYRALHRG
PAESVCKMTHGKTRWTQPQLICTGEMETSQFPGEEKPQASPEGRPESETS
CLVTTTDFQIQTEMAATMETSIFTTEYQVAVAGCVFLLISVLLLSGLTWQ
RRQRKSRRTI

In some embodiments, the chimeric transmembrane protein comprises or is a sequence that is at least 60% identical, at least 65% identical, at least 70% identical, at least 75% identical, at least 80% identical, at least 82% identical, at least 84% identical, at least 86% identical, at least 88% identical, at least 90% identical, at least 91% identical, at least 92% identical, at least 94% identical, at least 95% identical, at least 96% identical, at least 97% identical, at least 98% identical, at least 99% identical, or 100% identical to SEQ ID NO: 8.

In some embodiments, the chimeric transmembrane protein (e.g., mature or precursor protein) can be about 210 amino acids to about 650 amino acids, about 210 amino acids to about 640 amino acids, about 210 amino acids to about 620 amino acids, about 210 amino acids to about 600 amino acids, about 210 amino acids to about 580 amino acids, about 210 amino acids to about 560 amino acids, about 210 amino acids to about 540 amino acids, about 210 amino acids to about 520 amino acids, about 210 amino acids to about 500 amino acids, about 210 amino acids to about 480 amino acids, about 210 amino acids to about 460 amino acids, about 210 amino acids to about 440 amino acids, about 210 amino acids to about 420 amino acids, about 210 amino acids to about 400 amino acids, about 210 amino acids to about 380 amino acids, about 210 amino acids to about 360 amino acids, about 210 amino acids to about 340 amino acids, about 210 amino acids to about 320 amino acids, about 210 amino acids to about 300 amino acids, about 210 amino acids to about 280 amino acids, about 210 amino acids to about 260 amino acids, about 210 amino acids to about 240 amino acids, about 220 amino acids to about 650 amino acids, about 220 amino acids to about 640 amino acids, about 220 amino acids to about 620 amino acids, about 220 amino acids to about 600 amino acids, about 220 amino acids to about 580 amino acids, about 220 amino acids to about 560 amino acids, about 220 amino acids to about 540 amino acids, about 220 amino acids to about 520 amino acids, about 220 amino acids to about 500 amino acids, about 220 amino acids to about 480 amino acids, about 220 amino acids to about 460 amino acids, about 220 amino acids to about 440 amino acids, about 220 amino acids to about 420 amino acids, about 220 amino acids to about 400 amino acids, about 220 amino acids to about 380 amino acids, about 220 amino acids to about 360 amino acids, about 220 amino acids to about 340 amino acids, about 220 amino acids to about 320 amino acids, about 220 amino acids to about 300 amino acids, about 220 amino acids to about 280 amino acids, about 220 amino acids to about 260 amino acids, about 220 amino acids to about 240 amino acids, about 240 amino acids to about 650 amino acids, about 240 amino acids to about 640 amino acids, about 240 amino acids to about 620 amino acids, about 240 amino acids to about 600 amino acids, about 240 amino acids to about 580 amino acids, about 240 amino acids to about 560 amino acids, about 240 amino acids to about 540 amino acids, about 240 amino acids to about 520 amino acids, about 240 amino acids to about 500 amino acids, about 240 amino acids to about 480 amino acids, about 240 amino acids to about 460 amino acids, about 240 amino acids to about 440 amino acids, about 240 amino acids to about 420 amino acids, about 240 amino acids to about 400 amino acids, about 240 amino acids to about 380 amino acids, about 240 amino acids to about 360 amino acids, about 240 amino acids to about 340 amino acids, about 240 amino acids to about 320 amino acids, about 240 amino acids to about 300 amino acids, about 240 amino acids to about 280 amino acids, about 240 amino acids to about 260 amino acids, about 260 amino acids to about 650 amino acids, about 260 amino acids to about 640 amino acids, about 260 amino acids to about 620 amino acids, about 260 amino acids to about 600 amino acids, about 260 amino acids to about 580 amino acids, about 260 amino acids to about 560 amino acids, about 260 amino acids to about 540 amino acids, about 260 amino acids to about 520 amino acids, about 260 amino acids to about 500 amino acids, about 260 amino acids to about 480 amino acids, about 260 amino acids to about 460 amino acids, about 260 amino acids to about 440 amino acids, about 260 amino acids to about 420 amino acids, about 260 amino acids to about 400 amino acids, about 260 amino acids to about 380 amino acids, about 260 amino acids to about 360 amino acids, about 260 amino acids to about 340 amino acids, about 260 amino acids to about 320 amino acids, about 260 amino acids to about 300 amino acids, about 260 amino acids to about 280 amino acids, about 280 amino acids to about 650 amino acids, about 280 amino acids to about 640 amino acids, about 280 amino acids to about 620 amino acids, about 280 amino acids to about 600 amino acids, about 280 amino acids to about 580 amino acids, about 280 amino acids to about 560 amino acids, about 280 amino acids to about 540 amino acids, about 280 amino acids to about 520 amino acids, about 280 amino acids to about 500 amino acids, about 280 amino acids to about 480 amino acids, about 280 amino acids to about 460 amino acids, about 280 amino acids to about 440 amino acids, about 280 amino acids to about 420 amino acids, about 280 amino acids to about 400 amino acids, about 280 amino acids to about 380 amino acids, about 280 amino acids to about 360 amino acids, about 280 amino acids to about 340 amino acids, about 280 amino acids to about 320 amino acids, about 280 amino acids to about 300 amino acids, about 300 amino acids to about 650 amino acids, about 300 amino acids to about 640 amino acids, about 300 amino acids to about 620 amino acids, about 300 amino acids to about 600 amino acids, about 300 amino acids to about 580 amino acids, about 300 amino acids to about 560 amino acids, about 300 amino acids to about 540 amino acids, about 300 amino acids to about 520 amino acids, about 300 amino acids to about 500 amino acids, about 300 amino acids to about 480 amino acids, about 300 amino acids to about 460 amino acids, about 300 amino acids to about 440 amino acids, about 300 amino acids to about 420 amino acids, about 300 amino acids to about 400 amino acids, about 300 amino acids to about 380 amino acids, about 300 amino acids to about 360 amino acids, about 300 amino acids to about 340 amino acids, about 300 amino acids to about 320 amino acids, about 320 amino acids to about 650 amino acids, about 320 amino acids to about 640 amino acids, about 320 amino acids to about 620 amino acids, about 320 amino acids to about 600 amino acids, about 320 amino acids to about 580 amino acids, about 320 amino acids to about 560 amino acids, about 320 amino acids to about 540 amino acids, about 320 amino acids to about 520 amino acids, about 320 amino acids to about 500 amino acids, about 320 amino acids to about 480 amino acids, about 320 amino acids to about 460 amino acids, about 320 amino acids to about 440 amino acids, about 320 amino acids to about 420 amino acids, about 320 amino acids to about 400 amino acids, about 320 amino acids to about 380 amino acids, about 320 amino acids to about 360 amino acids, about 320 amino acids to about 340 amino acids, about 340 amino acids to about 650 amino acids, about 340 amino acids to about 640 amino acids, about 340 amino acids to about 620 amino acids, about 340 amino acids to about 600 amino acids, about 340 amino acids to about 580 amino acids, about 340 amino acids to about 560 amino acids, about 340 amino acids to about 540 amino acids, about 340 amino acids to about 520 amino acids, about 340 amino acids to about 500 amino acids, about 340 amino acids to about 480 amino acids, about 340 amino acids to about 460 amino acids, about 340 amino acids to about 440 amino acids, about 340 amino acids to about 420 amino acids, about 340 amino acids to about 400 amino acids, about 340 amino acids to about 380 amino acids, about 340 amino acids to about 360 amino acids, about 360 amino acids to about 650 amino acids, about 360 amino acids to about 640 amino acids, about 360 amino acids to about 620 amino acids, about 360 amino acids to about 600 amino acids, about 360 amino acids to about 580 amino acids, about 360 amino acids to about 560 amino acids, about 360 amino acids to about 540 amino acids, about 360 amino acids to about 520 amino acids, about 360 amino acids to about 500 amino acids, about 360 amino acids to about 480 amino acids, about 360 amino acids to about 460 amino acids, about 360 amino acids to about 440 amino acids, about 360 amino acids to about 420 amino acids, about 360 amino acids to about 400 amino acids, about 360 amino acids to about 380 amino acids, about 380 amino acids to about 650 amino acids, about 380 amino acids to about 640 amino acids, about 380 amino acids to about 620 amino acids, about 380 amino acids to about 600 amino acids, about 380 amino acids to about 580 amino acids, about 380 amino acids to about 560 amino acids, about 380 amino acids to about 540 amino acids, about 380 amino acids to about 520 amino acids, about 380 amino acids to about 500 amino acids, about 380 amino acids to about 480 amino acids, about 380 amino acids to about 460 amino acids, about 380 amino acids to about 440 amino acids, about 380 amino acids to about 420 amino acids, about 380 amino acids to about 400 amino acids, about 400 amino acids to about 650 amino acids, about 400 amino acids to about 640 amino acids, about 400 amino acids to about 620 amino acids, about 400 amino acids to about 600 amino acids, about 400 amino acids to about 580 amino acids, about 400 amino acids to about 560 amino acids, about 400 amino acids to about 540 amino acids, about 400 amino acids to about 520 amino acids, about 400 amino acids to about 500 amino acids, about 400 amino acids to about 480 amino acids, about 400 amino acids to about 460 amino acids, about 400 amino acids to about 440 amino acids, about 400 amino acids to about 420 amino acids, about 420 amino acids to about 650 amino acids, about 420 amino acids to about 640 amino acids, about 420 amino acids to about 620 amino acids, about 420 amino acids to about 600 amino acids, about 420 amino acids to about 580 amino acids, about 420 amino acids to about 560 amino acids, about 420 amino acids to about 540 amino acids, about 420 amino acids to about 520 amino acids, about 420 amino acids to about 500 amino acids, about 420 amino acids to about 480 amino acids, about 420 amino acids to about 460 amino acids, about 420 amino acids to about 440 amino acids, about 440 amino acids to about 650 amino acids, about 440 amino acids to about 640 amino acids, about 440 amino acids to about 620 amino acids, about 440 amino acids to about 600 amino acids, about 440 amino acids to about 580 amino acids, about 440 amino acids to about 560 amino acids, about 440 amino acids to about 540 amino acids, about 440 amino acids to about 520 amino acids, about 440 amino acids to about 500 amino acids, about 440 amino acids to about 480 amino acids, about 440 amino acids to about 460 amino acids, about 460 amino acids to about 650 amino acids, about 460 amino acids to about 640 amino acids, about 460 amino acids to about 620 amino acids, about 460 amino acids to about 600 amino acids, about 460 amino acids to about 580 amino acids, about 460 amino acids to about 560 amino acids, about 460 amino acids to about 540 amino acids, about 460 amino acids to about 520 amino acids, about 460 amino acids to about 500 amino acids, about 460 amino acids to about 480 amino acids, about 480 amino acids to about 650 amino acids, about 480 amino acids to about 640 amino acids, about 480 amino acids to about 620 amino acids, about 480 amino acids to about 600 amino acids, about 480 amino acids to about 580 amino acids, about 480 amino acids to about 560 amino acids, about 480 amino acids to about 540 amino acids, about 480 amino acids to about 520 amino acids, about 480 amino acids to about 500 amino acids, about 500 amino acids to about 650 amino acids, about 500 amino acids to about 640 amino acids, about 500 amino acids to about 620 amino acids, about 500 amino acids to about 600 amino acids, about 500 amino acids to about 580 amino acids, about 500 amino acids to about 560 amino acids, about 500 amino acids to about 540 amino acids, about 500 amino acids to about 520 amino acids, about 520 amino acids to about 650 amino acids, about 520 amino acids to about 640 amino acids, about 520 amino acids to about 620 amino acids, about 520 amino acids to about 600 amino acids, about 520 amino acids to about 580 amino acids, about 520 amino acids to about 560 amino acids, about 520 amino acids to about 540 amino acids, about 540 amino acids to about 650 amino acids, about 540 amino acids to about 640 amino acids, about 540 amino acids to about 620 amino acids, about 540 amino acids to about 600 amino acids, about 540 amino acids to about 580 amino acids, about 540 amino acids to about 560 amino acids, about 560 amino acids to about 650 amino acids, about 560 amino acids to about 640 amino acids, about 560 amino acids to about 620 amino acids, about 560 amino acids to about 600 amino acids, about 560 amino acids to about 580 amino acids, about 580 amino acids to about 650 amino acids, about 580 amino acids to about 640 amino acids, about 580 amino acids to about 620 amino acids, about 580 amino acids to about 600 amino acids, about 600 amino acids to about 650 amino acids, about 600 amino acids to about 640 amino acids, about 600 amino acids to about 620 amino acids, about 620 amino acids to about 650 amino acids, about 620 amino acids to about 640 amino acids, or about 630 amino acids to about 650 amino acids, in length.

In some embodiments, the chimeric transmembrane protein further comprises a signal sequence at its N-terminus. In some embodiments, the signal sequence comprises or is the sequence of MLLLVTSLLLCELPHPAFLLIP (SEQ ID NO: 9). Additional examples of signal sequences are known in the art. For example, a signal sequence can be about 5 amino acids to about 30 amino acids, about 5 amino acids to about 28 amino acids, about 5 amino acids to about 26 amino acids, about 5 amino acids to about 24 amino acids, about 5 amino acids to about 22 amino acids, about 5 amino acids to about 20 amino acids, about 5 amino acids to about 18 amino acids, about 5 amino acids to about 16 amino acids, about 5 amino acids to about 14 amino acids, about 5 amino acids to about 12 amino acids, about 5 amino acids to about 10 amino acids, about 5 amino acids to about 8 amino acids, about 6 amino acids to about 30 amino acids, about 6 amino acids to about 28 amino acids, about 6 amino acids to about 26 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 14 amino acids, about 6 amino acids to about 12 amino acids, about 6 amino acids to about 10 amino acids, about 6 amino acids to about 8 amino acids, about 8 amino acids to about 30 amino acids, about 8 amino acids to about 28 amino acids, about 8 amino acids to about 26 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 14 amino acids, about 8 amino acids to about 12 amino acids, about 8 amino acids to about 10 amino acids, about 10 amino acids to about 30 amino acids, about 10 amino acids to about 28 amino acids, about 10 amino acids to about 26 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 14 amino acids, about 10 amino acids to about 12 amino acids, about 12 amino acids to about 30 amino acids, about 12 amino acids to about 28 amino acids, about 12 amino acids to about 26 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 14 amino acids, about 14 amino acids to about 30 amino acids, about 14 amino acids to about 28 amino acids, about 14 amino acids to about 26 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 16 amino acids to about 30 amino acids, about 16 amino acids to about 28 amino acids, about 16 amino acids to about 26 amino acids, about 16 amino acids to about 24 amino acids, about 16 amino acids to about 22 amino acids, about 16 amino acids to about 20 amino acids, about 16 amino acids to about 18 amino acids, about 18 amino acids to about 30 amino acids, about 18 amino acids to about 28 amino acids, about 18 amino acids to about 26 amino acids, about 18 amino acids to about 24 amino acids, about 18 amino acids to about 22 amino acids, about 18 amino acids to about 20 amino acids, about 20 amino acids to about 30 amino acids, about 20 amino acids to about 28 amino acids, about 20 amino acids to about 26 amino acids, about 20 amino acids to about 24 amino acids, about 20 amino acids to about 22 amino acids, about 22 amino acids to about 30 amino acids, about 22 amino acids to about 28 amino acids, about 22 amino acids to about 26 amino acids, about 22 amino acids to about 24 amino acids, about 24 amino acids to about 30 amino acids, about 24 amino acids to about 28 amino acids, about 24 amino acids to about 26 amino acids, about 26 amino acids to about 30 amino acids, about 26 amino acids to about 28 amino acids, or about 28 amino acids to about 30 amino acids, in length.

In some embodiments, the chimeric transmembrane protein can further include a peptide tag. For example, a tag can be used to help facilitate purification, production, and/or identification of the chimeric transmembrane protein. In some embodiments, the tag is a histidine tag comprising at least six histidine residues. Additional examples of tags are known in the art.

Non-limiting aspects of any of the chimeric transmembrane proteins provided herein are described below.

### Extracellular IL-2 Domains

In some embodiments of any of the chimeric transmembrane proteins described herein, the extracellular IL-2 domain comprises or is a sequence of a wildtype IL-2 protein (e.g., a mature wildtype IL-2 protein). For example, the wildtype IL-2 protein can be a wildtype human IL-2 protein (e.g., a mature wildtype human IL-2 protein), a wildtype mouse IL-2 protein (e.g., a mature wildtype mouse IL-2 protein), a wildtype chimpanzee IL-2 protein (e.g., a mature wildtype chimpanzee IL-2 protein), or a wildtype monkey IL-2 protein (e.g., a mature wildtype monkey IL-2 protein). Non-limiting examples of wildtype IL-2 protein sequences and nucleic acids encoding these exemplary IL-2 protein sequences are provided below.

```
Mature Wildtype Human IL-2
(SEQ ID NO: 1)
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLT
cDNA encoding Mature Wildtype Human IL-2 Protein (mature protein
encoded by nucleotides 116 to 514 of SEQ ID NO: 10)
(SEQ ID NO: 10)
    1 agttccctat cactctcttt aatcactact cacagtaacc tcaactcctg ccacaatgta
  61 caggatgcaa ctcctgtctt gcattgcact aagtcttgca cttgtcacaa acagtgcacc
121 tacttcaagt tctacaaaga aaacacagct acaactggag catttactgc tggatttaca
181 gatgattttg aatggaatta ataattacaa gaatcccaaa ctcaccagga tgctcacatt
241 taagttttac atgcccaaga aggccacaga actgaaacat cttcagtgtc tagaagaaga
301 actcaaacct ctggaggaag tgctaaattt agctcaaagc aaaaactttc acttaagacc
361 cagggactta atcagcaata tcaacgtaat agttctggaa ctaaagggat ctgaaacaac
421 attcatgtgt gaatatgctg atgagacagc aaccattgta gaatttctga acagatggat
481 taccttttgt caaagcatca tctcaacact gacttgataa ttaagtgctt cccacttaaa
541 acatatcagg ccttctattt atttaaatat ttaaatttta tatttattgt tgaatgtatg
601 gtttgctacc tattgtaact attattctta atcttaaaac tataaatatg gatcttttat
661 gattcttttt gtaagcccta ggggctctaa aatggtttca cttatttatc ccaaaatatt
721 tattattatg ttgaatgtta aatatagtat ctatgtagat tggttagtaa aactatttaa
781 taaatttgat aaatataaaa aaaaaaaaaa aaaaaaaaaa aa
Mature Wildtype Monkey IL-2 Protein
(SEQ ID NO: 11)
aptssstkkt qlqlehllld lqmilnginn yknpkltrml tfkfympkka telkhlqcle eelkpleevl
nlaqsknfhl rdtkdlisni nvivlelkgs ettlmceyad etativefln rwitfcgsli stlt
cDNA encoding Mature Wildtype Monkey IL-2 Protein (mature protein
encoded by nucleotides 61 to 462 of SEQ ID NO: 12)
(SEQ ID NO: 12)
    1 atgtacagga tgcaactcct gtcttgcatt gcactaagtc ttgcacttgt cacaaacagt
  61 gcacctactt caagttctac aaagaaaaca cagctacaac tggagcattt actgctggat
121 ttacagatga ttttgaatgg aattaataat tacaagaatc ccaaactcac caggatgctc
181 acatttaagt tttacatgcc caagaaggcc acagaattga aacatcttca gtgtctagaa
241 gaagaactca aacctctgga ggaagtgcta aatttagctc aaagcaaaaa ctttcactta
301 agagatacca aggacttaat cagcaatatc aacgtaatag ttctggaact aaagggatct
361 gaaacaacac tgatgtgtga atatgctgat gagacagcaa ccattgtaga atttctgaac
421 agatggatta ccttttgtca aagcatcatc tcaacactga cctgataatt aagtgcttcc
481 cacttaaaac atatcag
Mature Wildtype Chicken IL-2 Protein
(SEQ ID NO: 13)
aslssekw ktlqtlikdl eileniknki hlelytptet qectqqtlqc ylgevvtlkk eteddtelke
efvtaignie knlksltgln htgseckice annkkkfpdf lheltnfvry lqk
cDNA encoding Mature Wildtype Chicken IL-2 Protein (mature protein
encoded by nucleotides 127 to 489 of SEQ ID NO: 14)
(SEQ ID NO: 14)
    1 atcacctcac attgtacatc acaatttgaa taccagcata cagataactg ggacactgcc
  61 atgatgtgca aagtactgat ctttggctgt atttcggtag caatgctaat gactacagct
121 tatggagcat ctctatcatc agaaaaatgg aaaactcttc aaacattaat aaaggattta
181 gaaatattgg aaaatatcaa gaataagatt catctcgagc tctacacacc aactgagacc
241 caggagtgca cccagcaaac tctgcagtgt tacctgggag aagtggttac tctgaagaaa
301 gaaactgaag atgacactga aattaaagaa gaatttgtaa ctgctattca aaatatcgaa
361 aagaacctca agagtcttac gggtctaaat cacaccggaa gtgaatgcaa gatctgtgaa
421 gctaacaaca agaaaaaatt tcctgatttt ctccatgaac tgaccaactt tgtgagatat
481 ctgcaaaaat aagcaactaa tcatttttat tttactgcta tgttatttat ttaattattt
541 aattacagat aatttatata ttttatcccg tggctaacta atctgctgtc cattctggga
601 ccactgtatg ctcttagtct gggtgatatg acgtctgttc taagatcata tttgatcctt
661 tctgtaagcc ctacgggctc aaaatgtacg ttggaaaact gattgattct cactttgtcg
721 gtaaagtgat atgtgtttac tgaaagaatt tttaaaagtc acttctagat gacatttaat
781 aaatttcagt aatatatg
Mature Wildtype Mouse IL-2 Protein
(SEQ ID NO: 15)
aptssstsss taeaqqqqqq qqqqqqhleq 11mdlgells rmenyrnlkl prmltfkfyl pkqatelkdl
qcledelgpl rhvldltqsk sfqledaenf isnirvtvvk lkgsdntfec qfddesatvy dflrrwiafc
qsiistspq
cDNA encoding Mature Wildtype Mouse IL-2 Protein (mature protein
encoded by nucleotides 109 to 555 of SEQ ID NO: 16)
(SEQ ID NO: 16)
    1 tatcaccctt gctaatcact cctcacagtg acctcaagtc ctgcaggcat gtacagcatg
  61 cagctcgcat cctgtgtcac attgacactt gtgctccttg tcaacagcgc acccacttca
121 agctccactt caagctctac agcggaagca cagcagcagc agcagcagca gcagcagcag
181 cagcagcacc tggagcagct gttgatggac ctacaggagc tcctgagcag gatggagaat
241 tacaggaacc tgaaactccc caggatgctc accttcaaat tttacttgcc caagcaggcc
301 acagaattga aagatcttca gtgcctagaa gatgaacttg gacctctgcg gcatgttctg
361 gatttgactc aaagcaaaag ctttcaattg gaagatgctg agaatttcat cagcaatatc
421 agagtaactg ttgtaaaact aaagggctct gacaacacat ttgagtgcca attcgatgat
481 gagtcagcaa ctgtggtgga ctttctgagg agatggatag ccttctgtca aagcatcatc
541 tcaacaagcc ctcaataact atgtacctcc tgcttacaac acataaggct ctctatttat
601 ttaaatattt aactttaatt tatttttgga tgtattgttt actatctttt gtaactacta
661 gtcttcagat gataaatatg gatctttaaa gattcttttt gtaagcccca agggctcaaa
721 aatgttttaa actatttatc tgaaattatt tattatattg aattgttaaa tatcatgtgt
781 aggtagactc attaataaaa gtatttagat gattcaaata taaataagct cagatgtctg
841 tcatttttag gacagcacaa agtaagcgct aaaataactt ctcagttatt cctgtgaact
901 ctatgttaat cagtgttttc aagaaataaa gctctcctct aaaaaaaaaa aaaaa
```

In some embodiments, the extracellular IL-2 domain comprises or is a sequence that is at least 60% identical, at least 65% identical, at least 70% identical, at least 75% identical, at least 80% identical, at least 82% identical, at least 84% identical, at least 85%, at least 86% identical, at least 88% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to a wildtype IL-2 protein sequence (e.g., any of the exemplary wildtype IL-2 protein sequences described herein or known in the art, e.g., SEQ ID NO: 1, 11, 13, or 15). In some embodiments, the wildtype IL-2 protein is a wildtype human IL-2 protein (e.g., a mature wildtype human IL-2 protein, e.g., SEQ ID NO: 1).

As one skilled in the art can appreciate, when amino acids that are not conserved between wildtype IL-2 proteins from different species are mutated (e.g., substituted with a different amino acid) they are less likely to cause a decrease in the level of one or more activities of a IL-2 protein. In contrast, when amino acids that are conserved between wildtype IL-2 proteins from different species are mutated (e.g., substituted with a different amino acid) they are more likely to cause a decrease in the level of one or more activities of a IL-2 protein. In view of this knowledge, one skilled in the art can select which amino acid positions in a wildtype IL-2 protein (e.g., the non-conserved amino acids) can be substituted without decreasing the activity of the IL-2 protein.

In some embodiments, the extracellular IL-2 domain is a sequence of a wildtype IL-2 protein (e.g., a mature wildtype IL-2 protein, e.g., any of the mature wildtype IL-2 proteins described herein, e.g., mature wildtype human IL-2 protein, e.g., SEQ ID NO: 1) having one to ten (e.g., one, two, three, four, five, six, seven, eight, nine, or ten) amino acids removed from the N-terminus of the sequence of the wildtype IL-2 protein. In some embodiments, the extracellular IL-2 domain is a sequence of a wildtype IL-2 protein (e.g., a mature wildtype IL-2 protein, e.g., any of the mature wildtype IL-2 proteins described herein, e.g., mature wildtype human IL-2 protein, e.g., SEQ ID NO: 1) having one to ten (e.g., one, two, three, four, five, six, seven, eight, nine, or ten) amino acids removed from the C-terminus of the sequence of the wildtype IL-2 protein. In some embodiments, the extracellular IL-2 domain is a sequence of a wildtype IL-2 protein (e.g., a mature wildtype IL-2 protein, e.g., any of the mature wildtype IL-2 proteins described herein, e.g., mature wildtype human IL-2 protein, e.g., SEQ ID NO: 1) having both one to ten amino (e.g., one, two, three, four, five, six, seven, eight, nine, or ten) acids removed from the N-terminus of the sequence of the wildtype IL-2 protein and one to ten (e.g., one, two, three, four, five, six, seven, eight, nine, or ten) amino acids removed from the C-terminus of the sequence of the wildtype IL-2 protein.

### Alpha Chain of an IL-2 Receptor

The IL-2 receptor is a heterotrimeric protein generated by different combinations of three different proteins-the alpha chain (IL-2Rα, also known as CD25), the beta chain (IL-2Rβ, also known as CD122), and the gamma chain (IL-2Rγc, also known as CD132).

The alpha chain (IL-2Rα) of human IL-2 receptor is encoded on chromosome 10p14-15 by the gene IL2RA. Non-limiting examples of mature wildtype alpha chains of IL-2 receptor are shown below. In some embodiments of any of the chimeric transmembrane proteins provided herein, the portion of the chimeric transmembrane protein that comprises the extracellular sushi domain and the transmembrane domain comprises or is a sequence of a mature wildtype alpha chain of IL-2 receptor (e.g., any of the exemplary mature wildtype alpha chain of IL-2 receptor protein sequences shown below, e.g., SEQ ID NO: 17).

| Mature Wildtype Human Alpha Chain of IL-2 Receptor | |
|---|---|
| (SEQ ID NO: 17) | |
| elcdddppe iphatfkama ykegtmlnce ckrgfrriks gslymlctgn sshsswdnqc qctssatrnt | |
| tkqvtpqpee qkerkttemq spmqpvdqas lpghcreppp weneateriy hfvvgqmvyy qcvqgyralh | |
| rgpaesvckm thgktrwtqp qlictgemet sqfpgeekpq aspegrpese tsclvtttdf qiqtemaatm | |
| etsiftteyq vavagcvfll isvlllsglt wqrrqrksrr ti | |
| cDNA Encoding Mature Wildtype Human Alpha Chain of IL-2 Receptor | |
| (mature protein encoded by nucleotides 283 to 1035 of SEQ ID NO: 18) | |
| (SEQ ID NO: 18) | |
| 1 | ggcagtttcc tggctgaaca cgccagccca atacttaaag agagcaactc ctgactccga |
| 601 | tagagactgg atggacccac aagggtgaca gcccaggcgg accgatcttc ccatcccaca |
| 121 | tcctccggcg cgatgccaaa aagaggctga cggcaactgg gccttctgca gagaaagacc |
| 181 | tccgcttcac tgccccggct ggtcccaagg gtcaggaaga tggattcata cctgctgatg |
| 241 | tggggactgc tcacgttcat catggtgcct ggctgccagg cagagctctg tgacgatgac |
| 301 | ccgccagaga tcccacacgc cacattcaaa gccatggcct acaaggaagg aaccatgttg |
| 361 | aactgtgaat gcaagagagg tttccgcaga ataaaaagcg ggtcactcta tatgctctgt |
| 421 | acaggaaact ctagccactc gtcctgggac aaccaatgtc aatgcacaag ctctgccact |
| 481 | cggaacacaa cgaaacaagt gacacctcaa cctgaagaac agaaagaaag gaaaaccaca |
| 541 | gaaatgcaaa gtccaatgca gccagtggac caagcgagcc ttccaggtca ctgcagggaa |
| 601 | cctccaccat gggaaaatga agccacagag agaatttatc atttcgtggt ggggcagatg |
| 661 | gtttattatc agtgcgtcca gggatacagg gctctacaca gaggtcctgc tgagagcgtc |
| 721 | tgcaaaatga cccacgggaa gacaaggtgg acccagcccc agctcatatg cacaggtgaa |
| 781 | atggagacca gtcagtttcc aggtgaagag aagcctcagg caagccccga aggccgtcct |
| 841 | gagagtgaga cttcctgcct cgtcacaaca acagattttc aaatacagac agaaatggct |
| 901 | gcaaccatgg agacgtccat atttacaaca gagtaccagg tagcagtggc cggctgtgtt |
| 961 | ttcctgctga tcagcgtcct cctcctgagt gggctcacct ggcagcggag acagaggaag |
| 1021 | agtagaagaa caatctagaa aaccaaaaga acaagaattt cttggtaaga agccgggaac |
| 1081 | agacaacaga agtcatgaag cccaagtgaa atcaaaggtg ctaaatggtc gcccaggaga |
| 1141 | catccgttgt gcttgcctgc gttttggaag ctctgaagtc acatcacagg acacggggca |
| 1201 | gtggcaacct tgtctctatg ccagctcagt cccatcagag agcgagcgct acccacttct |
| 1261 | aaatagcaat ttcgccgttg aagaggaagg gcaaaaccac tagaactctc catcttattt |
| 1321 | tcatgtatat gtgttcatta aagcatgaat ggtatggaac tctctccacc ctatatgtag |
| 1381 | tataaagaaa agtaggttta cattcatctc attccaactt cccagttcag gagtcccaag |
| 1441 | gaaagcccca gcactaacgt aaatacacaa cacacacact ctaccctata caactggaca |
| 1501 | ttgtctgcgt ggttcctttc tcagccgctt ctgactgctg attctcccgt tcacgttgcc |
| 1561 | taataaacat ccttcaagaa ctctgggctg ctacccagaa atcattttac ccttggctca |
| 1621 | atcctctaag ctaaccccct tctactgagc cttcagtctt gaatttctaa aaaacagagg |
| 1681 | ccatggcaga ataatctttg ggtaacttca aaacggggca gccaaaccca tgaggcaatg |
| 1741 | tcaggaacag aaggatgaat gaggtcccag gcagagaatc atacttagca aagttttacc |
| 1801 | tgtgcgttac taattggcct ctttaagagt tagtttcttt gggattgcta tgaatgatac |
| 1861 | cctgaatttg gcctgcacta atttgatgtt tacaggtgga cacacaaggt gcaaatcaat |
| 1921 | gcgtacgttt cctgagaagt gtctaaaaac accaaaaagg gatccgtaca ttcaatgttt |
| 1981 | atgcaaggaa ggaaagaaag aaggaagtga agagggagaa gggatggagg tcacactggt |
| 2041 | agaacgtaac cacggaaaag agcgcatcag gcctggcacg gtggctcagg cctataaccc |
| 2101 | cagctcccta ggagaccaag gcgggagcat ctcttgaggc caggagtttg agaccagcct |
| 2161 | gggcagcata gcaagacaca tccctacaaa aaattagaaa ttggctggat gtggtggcat |
| 2221 | acgcctgtag tcctagccac tcaggaggct gaggcaggag gattgcttga gcccaggagt |
| 2281 | tcgaggctgc agtcagtcat gatggcacca ctgcactcca gcctgggcaa cagagcaaga |
| 2341 | tcctgtcttt aaggaaaaaa agacaagatg agcataccag cagtccttga acattatcaa |
| 2401 | aaagttcagc atattagaat caccgggagg ccttgttaaa agagttcgct gggcccatct |
| 2461 | tcagagtctc tgagttgttg gtctggaata gagccaaatg ttttgtgtgt ctaacaattc |
| 2521 | ccaggtgctg ttgctgctgc tactattcca ggaacacact ttgagaacca ttgtgttatt |
| 2581 | gctctgcacg cccacccact ctcaactccc acgaaaaaaa tcaacttcca gagctaagat |
| 2641 | ttcggtggaa gtcctggttc catatctggt gcaagatctc ccctcacgaa tcagttgagt |
| 2701 | caacattcta gctcaacaac atcacacgat taacattaac gaaaattatt catttgggaa |
| 2761 | actatcagcc agttttcact tctgaagggg caggagagtg ttatgagaaa tcacggcagt |
| 2821 | tttcagcagg gtccagattc agattaaata actattttct gtcatttctg tgaccaacca |
| 2881 | catacaaaca gactcatctg tgcactctcc ccctccccct tcaggtatat gttttctgag |
| 2941 | taaagttgaa aagaatctca gaccagaaaa tatagatata tatttaaatc ttacttgagt |
| 3001 | agaactgatt acgacttttg ggtgttgagg ggtctataag atcaaaactt ttccatgata |
| 3061 | atactaagat gttatcgacc atttatctgt ccttctctca aaagtgtatg gtggaatttt |
| 3121 | ccagaagcta tgtgatacgt gatgatgtca tcactctgct gttaacatat aataaattta |
| 3181 | ttgctattgt ttataaaaga ataaatgata tttttt |
| Mature Wildtype Chimpanzee Alpha Chain of IL-2 Receptor | |
| (SEQ ID NO: 19) | |
| elcdddppe ithatfkama ykegtmlnce ckrgfrriks gslymlctgn sshsswdnqc qctssatrnt | |
| tkqvtpqpee qkerkttemq spmqpvdqas lpghcreppp weneateriy hfvvgqtvyy qcvqgyralh | |
| rgpaesvckm thgktrwtqp qlictgemet sqfpgeekpq aspegrpese tsclitttdf qiqtemaatm | |
| etfiftteyq vavagcvfll isvlllsglt wqrrq | |
| cDNA Encoding Mature Wildtype Chimpanzee Alpha Chain of IL-2 Receptor | |
| (mature protein encoded by nucleotides 64 to 798 of SEQ ID NO: 20) | |
| (SEQ ID NO: 20) | |
| 1 | atggattcat acctgctgat gtggggactg ctcacgctca tcatggtgcc tggctgcttt |
| 61 | gcagagctct gtgacgatga cccgccagag atcacacacg ccacattcaa agccatggcc |
| 121 | tacaaggaag gaaccatgtt gaactgtgaa tgcaagagag gtttccgcag aataaaaagc |
| 181 | gggtcactct atatgctctg tacaggaaac tctagccact cgtcctggga caaccaatgt |
| 241 | caatgcacaa gctctgccac tcggaacaca acgaaacaag tgacacctca acctgaagaa |
| 301 | cagaaagaaa ggaaaaccac agaaatgcaa agtccaatgc agccagtgga ccaagcgagc |
| 361 | cttccaggtc actgcaggga acctccaccg tgggaaaatg aagccacaga gagaatttat |
| 421 | catttcgtgg tggggcagac ggtttactac cagtgcgtcc agggatacag ggctctacac |
| 481 | agaggtcctg ctgagagcgt ctgcaaaatg acccatggga agacaaggtg gacccagccc |
| 541 | cagctcatat gcacaggtga aatggagacc agtcagtttc caggtgaaga gaagcctcag |
| 601 | gcaagccccg aaggccgtcc tgagagtgag acttcctgcc tcatcacaac aacagatttt |
| 661 | caaatacaga cagaaatggc tgcaaccatg gagacgttca tatttacaac agagtaccag |
| 721 | gtagcagtgg ccggctgtgt tttcctgctg atcagcgtcc tcctcctgag tgggctcacc |
| 781 | tggcagcgga gacagtaa |
| Mature Wildtype Monkey Alpha Chain of IL-2 Receptor | |
| (SEQ ID NO: 21) | |
| elcdddppk ithatfkava ykegtmlnce ckrgfrriks gspymlctgn sshsswdnqc qctssaarnt | |
| tkqvtpqpee qkerkttemq sqmqladqvs lpghcreppp weneateriy hfvvgqmvyy qcvqgyralh | |
| rgpaesickm thgktrwtqp qlictgetep sqfpgeeepq aspdglpese tsrlvtttdf riqtevaatm | |
| etfiftteyq vavagcvfll isvlllsglt wqrrqrknrr ti | |
| cDNA Encoding Mature Wildtype Monkey Alpha Chain of IL-2 Receptor | |
| (mature protein encoded by nucleotides 64 to 816 of SEQ ID NO: 22) | |
| (SEQ ID NO: 22) | |
| 1 | atggatccat acctgctcat gtggggactg ctcacgttca tcacggtgcc tggctgccag |
| 61 | gcagagctct gtgacgatga cccgccaaaa atcacacatg ccacattcaa agccgtggcc |
| 121 | tacaaggaag gaaccatgtt gaactgtgaa tgcaagagag gtttccgcag aataaaaagc |
| 181 | gggtcaccct atatgctctg tacaggaaac tctagccact cgtcctggga caaccaatgt |
| 241 | caatgcacaa gctctgctgc tcggaacaca acaaaacaag tgacacctca acctgaagaa |
| 301 | cagaaagaaa gaaaaaccac agaaatgcaa agtcaaatgc agctggcgga ccaagtgagc |
| 361 | cttccaggtc actgcaggga acctccaccg tgggaaaatg aagccacaga aagaatttat |
| 421 | catttcgtgg tggggcagat ggtttactac cagtgcgtcc agggatacag ggctctacac |
| 481 | agaggtcctg ctgagagcat ctgcaaaatg acccacggga agacaagatg gacccagccc |
| 541 | cagctcatat gcacaggtga aacggagccc agtcagtttc caggtgaaga ggagcctcag |
| 601 | gcaagccccg acggccttcc tgagagtgag acttcccgcc tcgtcacaac aacagatttt |
| 661 | cgaatacaga cagaagtggc tgcaaccatg gaaacgttca tatttacaac agagtaccaa |
| 721 | gtagcagtgg ccggctgtgt tttcctgctg atcagcgtcc tcctgctgag tgggctcacc |
| 781 | tggcagcgga gacagaggaa gaatagaaga acaatctaga aaaccaaaag aacaagaact |
| 841 | tcttggtaag aagccgagaa cagacaacag aagtcatgaa gcccaagcga aatcaaaggt |
| 901 | gctaaatgct tgcccaggag acatccgttg tgct |
| Mature Wildtype Mouse Alpha Chain of IL-2 Receptor | |
| (SEQ ID NO: 23) | |
| elclydppe vpnatfkals ykngtilnce ckrgfrrlke lvymrclgns wssncqctsn shdksrkqvt | |
| aqlehqkeqq tttdmqkptq smhqenltgh crepppwkhe dskriyhfve gqsvhyecip gykalqrgpa | |
| isickmkcgk tgwtqpqltc vderehhrfl aseesqgsrn sspesetscp itttdfpqpt ettamtetfv | |
| ltmeykvava sclfllisil llsgltwqhr wrksrrti | |
| eDNA Encoding Mature Wildtype Mouse Alpha Chain of IL-2 Receptor | |
| (mature protein encoded by nucleotides 273 to 1013 of SEQ ID NO: 24) | |
| (SEQ ID NO: 24) | |
| 1 | aagttcctgc tgagcagatc agcctaatgc ttaaatagaa caactcctgg ctgtcattga |
| 61 | cattgtctaa aagccaagat gacagactga gaggcctgag cccttgttct ggcattctcc |
| 121 | caggaagatg cagtaaaggg gttgacccaa tatactgcag agaatttcat ccagttccct |
| 181 | cctccatcct gatcccatgt gccaggaaga tggagccacg cttgctgatg ttggggtttc |
| 241 | tctcattaac catagtaccc agttgtcggg cagaactgtg tctgtatgac ccacccgagg |
| 301 | tccccaatgc cacattcaaa gccctctcct acaagaacgg caccatccta aactgtgaat |
| 361 | gcaagagagg tttccgaaga ctaaaggaat tggtctatat gcgttgctta ggaaactcct |
| 421 | ggagcagcaa ctgccagtgc accagcaact cccatgacaa atcgagaaag caagttacag |
| 481 | ctcaacttga acaccagaaa gagcaacaaa ccacaacaga catgcagaag ccaacacagt |
| 541 | ctatgcacca agagaacctt acaggtcact gcagggagcc acctccttgg aaacatgaag |
| 601 | attccaagag aatctatcat ttcgtggaag gacagagtgt tcactacgag tgtattccgg |
| 661 | gatacaaggc tctacagaga ggtcctgcta ttagcatctg caagatgaag tgtgggaaaa |
| 721 | cggggtggac tcagccccag ctcacatgtg tagatgaaag agaacaccac cgatttctgg |
| 781 | ctagtgagga atctcaagga agcagaaatt cttctcccga gagtgagact tcctgcccca |
| 841 | taaccaccac agacttccca caacccacag aaacaactgc aatgacggag acatttgtgc |
| 901 | tcacaatgga gtataaggta gcagtggcca gctgcctctt cctgctcatc agcatcctcc |
| 961 | tcctgagcgg gctcacctgg caacacagat ggaggaagag cagaagaacc atctagcaag |
| 1021 | ctagaaaagt cagagcccag gcaagcggat gggaatcaca aagctcaagc caaatctgag |
| 1081 | acgccaagca ttcacctaac ggctgtttcc ttctgatccc tgggtttcta gaacattctg |
| 1141 | aagtcacagg acataacagc aactctatca ctaaactgga ctttgccatt gaagaatagg |
| 1201 | atctaaccac ttcagcacag cagttctaaa gctttaatgg gagagagggc ccaacagtgc |
| 1261 | tctgtgtgtt ttgtttttgt gtatatctgt tgatgggagc tgagatggtg tggtcacttt |
| 1321 | tcatgtaaca tatagtatag aaaaagtagc tttaggttga cttcattgtt acaacccagt |
| 1381 | ttggaaagcc caagtaaaac tcagcactaa tgtaaataat tcctcctcct cctcctctct |
| 1441 | cttttcatcc tccgctccat cttcctcttc ttcctcctcc ttttccacct cctctgtccc |
| 1501 | tacccacccc cacccatcca ctttccttct tcctttctgc tctcacaagc tcatcctagc |
| 1561 | tacacgtgca tggctggctc ctttttcaac ctctgtttgc ctaactggct cttctgattt |
| 1621 | catcacttac tgatcagcct ttaaaactct gagctggcaa agatgactct atctatgttc |
| 1681 | ttggctcagt cccagaagga aacccccttt tcatgaagct tcagttttga catcctgaag |
| 1741 | aacagaaact gtggcagaac aatcttcaga taacatcaaa acaaagtgga gaagccacgg |
| 1801 | gaactgtgga gctctggtat tcagaagcct gtgtctaggg tctgcgccag gagcagaagg |
| 1861 | ctgaaggaag tcccaggacg tggacttaga tgctttccca gcaggccact ctaagcgctg |
| 1921 | gtttctttgg gacagctgtc aattgtacgc tcaatttagc ctgcactaat ctgatgctta |
| 1981 | caggtgaaca ctcaaggcac aggtatggac ttggtacata ccgtgaaaac actggaaaga |
| 2041 | aaagaatact ttcaagttta cagaaggaag gaaggaaaaa ggaagcagag gtggtgatta |
| 2101 | tacaaaagat tagctgtaga ctggatatcc caggcatcct cggataatgc ccccgcccca |
| 2161 | gcaccctgat ccaggtcacc aaagccttgt gagatcagac tgcagagcca gtctgtctct |
| 2221 | gagtcagtaa atgtagaatt tggatttctc acaagttcct ggcggtgtct tttttttttt |
| 2281 | taatattttt tattaggtat tttcctcatt tacatttcca atgctatccc aaaagtcccc |
| 2341 | cataccctcc cccacactcc cctacccacc cactcccact ttttggccct ggtgttcccc |
| 2401 | tgtactgggg catataaagt ttgcaagtcc tggtggtgtc tttatgctga tctctagccc |
| 2461 | acactttgtg aggcactggg ctatcccagt gtgctctcct cttccacaga taccaaaagc |
| 2521 | acctgggttt gatgctcaga cttctgagca cgttcttgtt caatctcttg cgtaagattt |
| 2581 | cctctcagat gagttgagtc agattctcat gtttaacagt gttttagggg attcacagaa |
| 2641 | gcccaaacta tcagttttca tttctgaaaa ggctggaaaa ttttatgaaa aactttcaaa |
| 2701 | ggtcagacag agccattttg agtcttttat gtgaccaagt atgaaccaga tctttcctat |
| 2761 | ctatggtctc ccctttccaa aatatatctt ttgtggggac acggcaagga ggaaagttaa |
| 2821 | atagaatctc aagctactaa ttttagaaaa gaaaaaaata ttaaactctt actaaagagc |
| 2881 | tgtgggtagt ggtacacacc tgtaagccca gctctcagga gactgaggca ggaggattgc |
| 2941 | agtgagtcag agatcagctt cacctacaag caaaaccctg ccttacccct caacctttcc |
| 3001 | ataaaaacag tcttacttgt gtaaaattaa tttttaatac atatttgtgc acgatgtgtg |
| 3061 | tgcctggtgc ccagggaagc caaaaaaata tgttcaatgt ccctggaata ggtggttatg |
| 3121 | agctgccatg gaggtgatgg gatttaaacc cctgttctct gaaaaagcag ctagttctct |
| 3181 | taaccactga gccatctcta cagccccatt aaattgaatt ttattgtcat tactcaatat |
| 3241 | gggagatggg gtaatgataa caattttttt tttataatac taagatgttt agctatttta |
| 3301 | ctctccttca caagtgtaga gtagaatttt ctagaagcta catgcatgat attatcgctc |
| 3361 | tggtgttaac acataatgga tttatcttgt taataaaaga attagtaaat aatattttaa |
| 3421 | attttttctt tgttttagtt ttaagatgat taatatctat agatactagt gtacattaag |
| 3481 | aaagcctttg ggatcctcaa tcattttgca tggttttagt aattttttaa taacataaag |
| 3541 | aaggtctgac agattatgct aaagagctat tgtggtatgg attagaaatg gcccccacag |
| 3601 | gctcctgtgt tcaaacatca gctagcagtg ctgctttggg ttttggaacc tttaagaggt |
| 3661 | ggggccttgc ttaaggagat aggtcactgg aggtgaacca gacttgcttc cagtctccct |
| 3721 | ctctgctgtc ccaggaagcc atcatatgag gagtttcaca acatacttct gctgccacag |
| 3781 | agtccctcca gtaaggcacg tactggccac tgtgtagtgt accctctaaa actgagctag |
| 3841 | agctcccctc tcctccctac actatctctg ttgatgcttt gtcacggtga tgaagaacat |
| 3901 | aagtaaggca aagacaagac atagtttgga gactcacgtg agcatctcag ccagactcag |
| 3961 | gcacagctgc gatgtgggaa ttatcaagca tgagatgcaa agcaatggaa aatgaatcgt |
| 4021 | tatgacagaa gcctacatct agtcttccct tcttcccatt agtaataata gcccgtgttt |
| 4081 | tagaagaaca catctttttg gtgttctagg tagcttatat tgcaaatgtg gcacaatcta |
| 4141 | agagaaatct gggatgaggg aacctcagtg aaagattctc cttgatcaga ttgtcccact |
| 4201 | gatgattgat atgggaaggc ccaacccact gtgaaaggca ccacccattg gcaggatgac |
| 4261 | tggggttata gaagcaaaca ggctgagcat gagccagtga gcaagccagt aagcagtggc |
| 4321 | ttctctttgg aagaagcatg gctttcttcc gtggcttctg tttgatctct ctcaatgata |
| 4381 | gattgtgacc tagaagtata agctaaaata aaccatttct tacccata |

In some embodiments of any of the chimeric transmembrane proteins provided herein, the portion of the chimeric transmembrane protein that comprises the extracellular sushi domain and the transmembrane domain comprises or is a sequence that is at least 60% identical, at least 65% identical, at least 70% identical, at least 75% identical, at least 80% identical, at least 82% identical, at least 84% identical, at least 85%, at least 86% identical, at least 88% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to a wildtype alpha chain of IL-2 receptor protein sequence (e.g., any of the exemplary mature wildtype alpha chain of IL-2 receptor protein sequences described herein or known in the art, e.g., SEQ ID NO: 17, 19, 21, or 23). In some embodiments of any of the chimeric transmembrane proteins described herein, the portion of the chimeric transmembrane protein that comprises the extracellular sushi domain and the transmembrane domain comprises or is a wildtype human alpha chain of IL-2 receptor protein (e.g., a mature wildtype human alpha chain of IL-2 receptor protein, e.g., SEQ ID NO: 17).

As one skilled in the art can appreciate, when amino acids that are not conserved between wildtype alpha chains of IL-2 receptor protein from different species are mutated (e.g., substituted with a different amino acid) they are less likely to cause a decrease in the level of one or more activities of an alpha chain of IL-2 receptor protein. In contrast, when amino acids that are conserved between wildtype alpha chains of IL-2 receptor protein from different species are mutated (e.g., substituted with a different amino acid) they are more likely to cause a decrease in the level of one or more activities of an alpha chain of IL-2 receptor protein. In view of this knowledge, one skilled in the art can select which amino acid positions in a wildtype alpha chain of an IL-2 receptor protein (e.g., the non-conserved amino acids) can be substituted without decreasing the activity of the alpha chain of an IL-2 receptor protein.

In some embodiments, the portion of the chimeric transmembrane protein that comprises the extracellular sushi domain and the transmembrane domain comprises or is a sequence of a wildtype alpha chain of IL-2 receptor protein (e.g., a mature wildtype alpha chain of IL-2 receptor protein, e.g., any of the mature wildtype alpha chains of IL-2 receptor described herein, e.g., mature wildtype human alpha chain of IL-2 receptor protein, e.g., SEQ ID NO: 17) having one to ten (e.g., one, two, three, four, five, six, seven, eight, nine, or ten) amino acids removed from the N-terminus of the sequence of the wildtype alpha chain of IL-2 receptor protein. In some embodiments, the portion of the chimeric transmembrane protein that comprises the extracellular sushi domain and the transmembrane domain comprises or is a sequence of a wildtype alpha chain of IL-2 receptor protein (e.g., a mature wildtype alpha chain of IL-2 receptor protein, e.g., any of the mature wildtype alpha chains of IL-2 receptor described herein, e.g., mature wildtype human alpha chain of IL-2 receptor protein, e.g., SEQ ID NO: 17) having one to ten (e.g., one, two, three, four, five, six, seven, eight, nine, or ten) amino acids removed from the C-terminus of the sequence of the wildtype alpha chain of IL-2 receptor protein. In some embodiments, the portion of the chimeric transmembrane protein that comprises the extracellular sushi domain and the transmembrane domain comprises or is a sequence of a wildtype alpha chain of IL-2 receptor protein (e.g., a mature wildtype alpha chain of IL-2 receptor protein, e.g., any of the mature wildtype alpha chains of IL-2 receptor described herein, e.g., mature wildtype human alpha chain of IL-2 receptor protein, e.g., SEQ ID NO: 17) having both one to ten amino (e.g., one, two, three, four, five, six, seven, eight, nine, or ten) acids removed from the N-terminus of the sequence of the wildtype alpha chain of IL-2 receptor protein and one to ten (e.g., one, two, three, four, five, six, seven, eight, nine, or ten) amino acids removed from the C-terminus of the sequence of the wildtype alpha chain of IL-2 receptor protein.

### Extracellular Sushi Domain of an Alpha Chain of IL-2 Receptor

In some embodiments, the chimeric transmembrane receptors described herein can include one or more (e.g., one, two, three, or four) sushi domains from an alpha chain of IL-2 receptor. A sushi domain, also known as a short consensus repeat or type 1 glycoprotein motif, is a common motif in protein-protein interaction. Sushi domains have been identified on a number of protein-binding molecules, including complement components C1r, C1s, factor H, and C2m, as well as the nonimmunologic molecules factor XIII and B2-glycoprotein. A typical Sushi domain has approximately 60 amino acid residues and contains four cysteines (Ranganathan, Pac. Symp Biocomput. 2000:155-67). The first cysteine can form a disulfide bond with the third cysteine, and the second cysteine can form a disulfide bridge with the fourth cysteine.

Non-limiting examples of sushi domains are described herein. For example, a sushi domain can be a sushi domain from a wildtype alpha chain of IL-2 receptor (e.g., a sushi domain from a wildtype human alpha chain of IL-2 receptor).

For example, a sushi domain can be about 25 amino acids to about 90 amino acids, about 25 amino acids to about 85 amino acids, about 25 amino acids to about 80 amino acids, about 25 amino acids to about 75 amino acids, about 25 amino acids to about 70 amino acids, about 25 amino acids to about 65 amino acids, about 25 amino acids to about 60 amino acids, about 25 amino acids to about 55 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 35 amino acids to about 90 amino acids, about 35 amino acids to about 85 amino acids, about 35 amino acids to about 80 amino acids, about 35 amino acids to about 75 amino acids, about 35 amino acids to about 70 amino acids, about 35 amino acids to about 65 amino acids, about 35 amino acids to about 60 amino acids, about 35 amino acids to about 55 amino acids, about 35 amino acids to about 50 amino acids, about 35 amino acids to about 45 amino acids, about 35 amino acids to about 40 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 45 amino acids to about 90 amino acids, about 45 amino acids to about 85 amino acids, about 45 amino acids to about 80 amino acids, about 45 amino acids to about 75 amino acids, about 45 amino acids to about 70 amino acids, about 45 amino acids to about 65 amino acids, about 45 amino acids to about 60 amino acids, about 45 amino acids to about 55 amino acids, about 45 amino acids to about 50 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 55 amino acids to about 90 amino acids, about 55 amino acids to about 85 amino acids, about 55 amino acids to about 80 amino acids, about 55 amino acids to about 75 amino acids, about 55 amino acids to about 70 amino acids, about 55 amino acids to about 65 amino acids, about 55 amino acids to about 60 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 65 amino acids to about 90 amino acids, about 65 amino acids to about 85 amino acids, about 65 amino acids to about 80 amino acids, about 65 amino acids to about 75 amino acids, about 65 amino acids to about 70 amino acids, about 70 amino acids to about 90 amino acids, about 70 amino acids to about 85 amino acids, about 70 amino acids to about 80 amino acids, about 70 amino acids to about 75 amino acids, about 75 amino acids to about 90 amino acids, about 75 amino acids to about 85 amino acids, about 75 amino acids to about 80 amino acids, about 80 amino acids to about 90 amino acids, about 80 amino acids to about 85 amino acids, or about 85 amino acids to about 90 amino acids, in length.

For example, an extracellular sushi domain comprises or is an extracellular portion of a mature wildtype alpha chain of IL-2 receptor, e.g., a mature wildtype human alpha chain of IL-2 receptor, e.g., SEQ ID NO: 25 (shown below).

Extracellular Portion of Mature Wildtype Human
Alpha Chain of IL-2 Receptor
(SEQ ID NO: 25)
elcdddppe iphatfkama ykegtmlnce ckrgfrriks
gslymlctgn sshsswdnqc qctssatrnt tkqvtpqpee
qkerkttemq spmqpvdqas lpghcreppp weneateriy
hfvvgqmvyy qcvqgyralh rgpaesvckm thgktrwtqp
qlictgemet sqfpgeekpq aspegrpese tsclvtttdf
qiqtemaatm etsiftteyq

In some embodiments, an extracellular sushi domain comprises or is a sequence that is at least 60% identical, at least 65% identical, at least 70% identical, at least 75% identical, at least 80% identical, at least 82% identical, at least 84% identical, at least 85%, at least 86% identical, at least 88% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to an extracellular portion of a mature wildtype alpha chain of IL-2 receptor, e.g., a mature wildtype human alpha chain of IL-2 receptor, e.g., SEQ ID NO: 25.

In some embodiments, an extracellular sushi domain is an extracellular portion of a mature wildtype alpha chain of IL-2 receptor (e.g., a mature wildtype human alpha chain of IL-2 receptor, e.g., SEQ ID NO: 25) having one to twenty (e.g., 1, 2, 3, 4, 5, 6, 7, 8, 9, 10, 11, 12, 13, 14, 15, 16, 17, 18, 19, or 20) amino acids removed from the N-terminus of the sequence of the extracellular portion of the mature wildtype alpha chain of IL-2 receptor. In some embodiments, an extracellular sushi domain is an extracellular portion of a mature wildtype alpha chain of IL-2 receptor (e.g., a mature wildtype human alpha chain of IL-2 receptor, e.g., SEQ ID NO: 25) having one to twenty (e.g., 1, 2, 3, 4, 5, 6, 7, 8, 9, 10, 11, 12, 13, 14, 15, 16, 17, 18, 19, or 20) amino acids removed from the C-terminus of the sequence of the extracellular portion of the mature wildtype alpha chain of IL-2 receptor. In some embodiments, an extracellular sushi domain is an extracellular portion of a mature wildtype alpha chain of IL-2 receptor (e.g., a mature wildtype human alpha chain of IL-2 receptor, e.g., SEQ ID NO: 25) having one to twenty (e.g., 1, 2, 3, 4, 5, 6, 7, 8, 9, 10, 11, 12, 13, 14, 15, 16, 17, 18, 19, or 20) amino acids removed from the N-terminus of the sequence of the extracellular portion of the mature wildtype alpha chain of IL-2 receptor and one to twenty (e.g., 1, 2, 3, 4, 5, 6, 7, 8, 9, 10, 11, 12, 13, 14, 15, 16, 17, 18, 19, or 20) amino acids removed from the C-terminus of the sequence of the extracellular portion of the mature wildtype alpha chain of IL-2 receptor.

In some embodiments, an extracellular sushi domain comprises or is a sequence of a sushi domain from a wildtype alpha-chain of IL-2 receptor (e.g., any of the sushi domains from wildtype alpha-chains of IL-2 receptor listed below, e.g., a sushi domain from wildtype human alpha-chain of IL-2 receptor, e.g., a sequence comprising one or both of SEQ ID NO: 3 and 4 below).

Exemplary Sushi Domain from Wildtype Human Alpha
Chain of IL-2 Receptor
(SEQ ID NO: 3)
elcdddppeiphatfkamaykegtmlnceckrgfrriksgslymlctgnss
hsswdnqc qcts
Exemplary Sushi Domain from Wildtype Human Alpha
Chain of IL-2 Receptor
(SEQ ID NO: 4)
ghcrepppweneateriyhfvvgqmvyyqcvqgyralhrgpaesvckmthg
ktrwtqpqlictg
Exemplary Sushi Domain from Wildtype Chimpanzee
Alpha Chain of IL-2 Receptor
(SEQ ID NO: 26)
elcdddppeithatfkamaykegtmlnceckrgfrriksgslymlctgnss
hsswdnqc qcts
Exemplary Sushi Domain from Wildtype Chimpanzee
Alpha Chain of IL-2 Receptor
(SEQ ID NO: 27)
ghcrepppweneateriyhfvvgqtvyyqcvqgyralhrgpaesvckmthg
ktrwtqpqlictg
Exemplary Sushi Domain from Wildtype Monkey Alpha
Chain of IL-2 Receptor
(SEQ ID NO: 28)
elcdddppkithatfkavaykegtmlnceckrgfrriksgspymlctgnss
hsswdnqc qcts
Exemplary Sushi Domain from Wildtype Monkey Alpha
Chain of IL-2 Receptor
(SEQ ID NO: 29)
ghcrepppweneateriyhfvvgqmvyyqcvqgyralhrgpaesickmthg
ktrwtqpqlictg
Exemplary Sushi Domain from Wildtype Mouse Alpha
Chain of IL-2 Receptor
(SEQ ID NO: 30)
elclydppevpnatfkalsykngtilnceckrgfrrlkelvymrclgnsws
sncqcts
Exemplary Sushi Domain from Wildtype Mouse Alpha
Chain of IL-2 Receptor
(SEQ ID NO: 31)
ghcrepppwkhedskriyhfveggsvhyecipgykalqrgpaisickmkcg
ktgwtqpqltcvd

In some embodiments, the extracellular sushi domain of the chimeric transmembrane protein comprises a sequence that is at least 60% identical, at least 65% identical, at least 70% identical, at least 75% identical, at least 80% identical, at least 82% identical, at least 84% identical, at least 85%, at least 86% identical, at least 88% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to a sushi domain of a wildtype alpha chain of IL-2 receptor (e.g., SEQ ID NO: 3, 4, 26, 27, 28, 29, 30, or 31).

As one skilled in the art can appreciate, when amino acids that are not conserved between sushi domains from wildtype alpha chains of IL-2 receptor protein from different species are mutated (e.g., substituted with a different amino acid) they are less likely to cause a decrease in the level of IL-2 binding activity of the sushi domain from an alpha chain of IL-2 receptor protein. In contrast, when amino acids that are conserved between the sushi domains from wildtype alpha chains of IL-2 receptor protein from different species are mutated (e.g., substituted with a different amino acid) they are more likely to cause a decrease in the level of IL-2 binding activity of the sushi domain from an alpha chain of IL-2 receptor protein. In view of this knowledge, one skilled in the art can select which amino acid positions in a sushi domain of an alpha chain of an IL-2 receptor protein (e.g., the non-conserved amino acids) can be substituted without decreasing the activity of the sushi domain of the alpha chain of an IL-2 receptor protein.

In some embodiments, the extracellular sushi domain of the chimeric transmembrane protein is a sequence of a sushi domain from a wildtype alpha chain of an IL-2 receptor (e.g., any of SEQ ID NO: 3, 4, 26, 27, 28, 29, 30, or 31) having one to five (e.g., one, two, three, four, or five) amino acids removed from the N-terminus of the sushi domain from the wildtype alpha chain of an IL-2 receptor. In some embodiments, the extracellular sushi domain of the chimeric transmembrane protein is a sequence of a sushi domain from a wildtype alpha chain of an IL-2 receptor (e.g., any of SEQ ID NO: 3, 4, 26, 27, 28, 29, 30, or 31) having one to five (e.g., one, two, three, four, or five) amino acids removed from the C-terminus of the sushi domain from the wildtype alpha chain of an IL-2 receptor. In some embodiments, the extracellular sushi domain of the chimeric transmembrane protein is a sequence of a sushi domain from a wildtype alpha chain of an IL-2 receptor (e.g., any of SEQ ID NO: 3, 4, 26, 27, 28, 29, 30, or 31) having one to five (e.g., one, two, three, four, or five) amino acids removed from the N-terminus of the sushi domain from the wildtype alpha chain of an IL-2 receptor, and one to five (e.g., one, two, three, four, or five) amino acids removed from the C-terminus of the sushi domain from the wildtype alpha chain of an IL-2 receptor.

In some embodiments, the chimeric tansmembrane protein comprises two sushi domains. In some embodiments, each of the sushi domains can independently be any of the sushi domains described herein. In some embodiments, one of the two sushi domains comprises or is SEQ ID NO: 3, and the other of the two sushi domains comprises or is SEQ ID NO: 4.

### Transmembrane Domains of an Alpha-Chain of an IL-2 Receptor

In some embodiments, the transmembrane domain from an alpha chain of an IL-2 receptor protein can be about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 22 amino acids, about 10 amino acids to about 20 amino acids, about about 10 amino acids to about 18 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 22 amino acids, about 15 amino acids to about 20 amino acids, about about 15 amino acids to about 18 amino acids, about 18 amino acids to about 50 amino acids, about 18 amino acids to about 45 amino acids, about 18 amino acids to about 40 amino acids, about 18 amino acids to about 35 amino acids, about 18 amino acids to about 30 amino acids, about 18 amino acids to about 25 amino acids, about 18 amino acids to about 22 amino acids, about 18 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 20 amino acids to about 22 amino acids, about 22 amino acids to about 50 amino acids, about 22 amino acids to about 45 amino acids, about 22 amino acids to about 40 amino acids, about 22 amino acids to about 35 amino acids, about 22 amino acids to about 30 amino acids, about 22 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 35 amino acids to about 50 amino acids, about 35 amino acids to about 45 amino acids, about 35 amino acids to about 40 amino acids, about 40 amino acids to about 50 amino acids, about 40 amino acids to about 45 amino acids, or about 45 amino acids to about 50 amino acids, in length.

In some embodiments of any of the chimeric transmembrane proteins described herein, the transmembrane domain of an alpha chain of an IL-2 receptor can be a transmembrane domain from a wildtype alpha chain of an IL-2 receptor (e.g., a transmembrane domain of a wildtype human alpha chain of an IL-2 receptor, e.g., SEQ ID NO: 5) (e.g., any of the exemplary transmembrane domains of a wildtype alpha chain of an IL-2 receptor listed below or known in the art, e.g., SEQ ID NO: 32, 33, or 34).

Exemplary Transmembrane Domain of Wildtype Human
Alpha Chain of IL-2 Receptor
(SEQ ID NO: 5)
VAVAGCVFLLISVLLLSGL
Exemplary Transmembrane Domain of Wildtype
Chimpanzee Alpha Chain of IL-2 Receptor
(SEQ ID NO: 32)
Vavagcvfllisvillsgl
Exemplary Transmembrane Domain of Wildtype Monkey
Alpha Chain of IL-2 Receptor
(SEQ ID NO: 33)
vavagcvfllisvlllsgl
Exemplary Transmembrane Domain of Wildtype Mouse
Alpha Chain of IL-2 Receptor
(SEQ ID NO: 34)
vavasclfllisilllsgltw

In some embodiments, the transmembrane domain of an alpha chain of IL-2 receptor comprises or is a sequence that is at least 60% identical, at least 65% identical, at least 70% identical, at least 75% identical, at least 80% identical, at least 82% identical, at least 84% identical, at least 85%, at least 86% identical, at least 88% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to a transmembrane domain of a wildtype alpha chain of IL-2 receptor (e.g., SEQ ID NO: 5, 32, 33, or 34).

As one skilled in the art can appreciate, when amino acids that are not conserved between transmembrane domains from wildtype alpha chains of IL-2 receptor protein from different species are mutated (e.g., substituted with a different amino acid) they are less likely to cause a decrease in the activity of the transmembrane domain from an alpha chain of IL-2 receptor protein. In contrast, when amino acids that are conserved between the transmembrane domains from wildtype alpha chains of IL-2 receptor protein from different species are mutated (e.g., substituted with a different amino acid) they are more likely to cause a decrease in the activity of the transmembrane domain from an alpha chain of IL-2 receptor protein. In view of this knowledge, one skilled in the art can select which amino acid positions in a transmembrane domain of an alpha chain of an IL-2 receptor protein (e.g., the non-conserved amino acids) can be substituted without decreasing the activity of the transmembrane domain from the alpha chain of an IL-2 receptor protein.

In some embodiments, the transmembrane domain of an alpha chain of an IL-2 receptor protein is a sequence of a transmembrane domain of a wildtype alpha chain of IL-2 receptor protein (e.g., a transmembrane domain of a wildtype human alpha chain of IL-2 receptor protein, e.g., SEQ ID NO: 5) (e.g., SEQ ID NO: 32, 33, or 34) having one to five (e.g., one, two, three, four, or five) amino acids removed from the N-terminus of the transmembrane domain of the alpha chain of the IL-2 receptor protein. In some embodiments, the transmembrane domain of an alpha chain of an IL-2 receptor protein is a sequence of a transmembrane domain of a wildtype alpha chain of an IL-2 receptor protein (e.g., a transmembrane domain of a wildtype human alpha chain of IL-2 receptor protein, e.g., SEQ ID NO: 5) (e.g., SEQ ID NO: 32, 33, or 34) having one to five (e.g., one, two, three, four, or five) amino acids removed from the C-terminus of the transmembrane domain of the alpha chain of the IL-2 receptor protein. In some embodiments, the transmembrane domain of an alpha chain of an IL-2 receptor protein is a sequence of a transmembrane domain of a wildtype alpha chain of IL-2 receptor protein (e.g., a transmembrane domain of a wildtype human alpha chain of IL-2 receptor protein, e.g., SEQ ID NO: 5) (e.g., SEQ ID NO: 32, 33, or 34) having one to five (e.g., one, two, three, four, or five) amino acids removed from the N-terminus of the transmembrane domain of the alpha chain of the IL-2 receptor protein, and one to five (e.g., one, two, three, four, or five) amino acids removed from the C-terminus of the transmembrane domain of the alpha chain of the IL-2receptor protein.

Additional examples of transmembrane domains from an alpha chain of an IL-2 receptor protein are known in the art.

### Intracellular Domains of an Alpha-chain of IL-2 Receptor

Any of the chimeric transmembrane proteins described herein can comprise an intracellular domain of an alpha chain of an interleukin-2 receptor (e.g., any of the alpha chains of an interleukin-2 receptor described herein). In some embodiments, the intracellular domain of an alpha chain of an interleukin-2 receptor can be about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 20 amino acids, about 8 amino acids to about 18 amino acids, about 8 amino acids to about 16 amino acids, about 8 amino acids to about 14 amino acids, about 8 amino acids to about 12 amino acids, about 8 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 18 amino acids, about 10 amino acids to about 16 amino acids, about 10 amino acids to about 14 amino acids, about 10 amino acids to about 12 amino acids, about 12 amino acids to about 50 amino acids, about 12 amino acids to about 45 amino acids, about 12 amino acids to about 40 amino acids, about 12 amino acids to about 35 amino acids, about 12 amino acids to about 30 amino acids, about 12 amino acids to about 25 amino acids, about 12 amino acids to about 20 amino acids, about 12 amino acids to about 18 amino acids, about 12 amino acids to about 16 amino acids, about 12 amino acids to about 14 amino acids, about 14 amino acids to about 50 amino acids, about 14 amino acids to about 45 amino acids, about 14 amino acids to about 40 amino acids, about 14 amino acids to about 35 amino acids, about 14 amino acids to about 30 amino acids, about 14 amino acids to about 25 amino acids, about 14 amino acids to about 20 amino acids, about 14 amino acids to about 18 amino acids, about 14 amino acids to about 16 amino acids, about 16 amino acids to about 50 amino acids, about 16 amino acids to about 45 amino acids, about 16 amino acids to about 40 amino acids, about 16 amino acids to about 35 amino acids, about 16 amino acids to about 30 amino acids, about 16 amino acids to about 25 amino acids, about 16 amino acids to about 20 amino acids, about 16 amino acids to about 18 amino acids, about 18 amino acids to about 50 amino acids, about 18 amino acids to about 45 amino acids, about 18 amino acids to about 40 amino acids, about 18 amino acids to about 35 amino acids, about 18 amino acids to about 30 amino acids, about 18 amino acids to about 25 amino acids, about 18 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 35 amino acids to about 50 amino acids, about 35 amino acids to about 45 amino acids, about 35 amino acids to about 40 amino acids, about 40 amino acids to about 50 amino acids, about 40 amino acids to about 45 amino acids, or about 45 amino acids to about 50 amino acids, in length.

Any of the chimeric transmembrane proteins described herein can comprise an intracellular domain of an alpha chain of interleukin-2 receptor (e.g., an intracellular domain of a wildtype alpha chain of interleukin-2 receptor, e.g., an intracellular domain of a wildtype human alpha chain of interleukin-2 receptor, e.g., SEQ ID NO: 6) (e.g., SEQ ID NO: 35, 36, or 37).

Exemplary Intracellular Domain of Wildtype Human
Alpha Chain of IL-2 Receptor
(SEQ ID NO: 6)
TWQRRQRKSRRTI
Exemplary Intracellular Domain of Wildtype
Chimpanzee Alpha Chain of IL-2 Receptor
(SEQ ID NO: 35)
twqrrq
Exemplary Intracellular Domain of Wildtype Monkey
Alpha Chain of IL-2 Receptor
(SEQ ID NO: 36)
twqrrqrknrrti
Exemplary Intracellular Domain of Wildtype Mouse
Alpha Chain of IL-2 Receptor
(SEQ ID NO: 37)
qhrwrksrrti

In some embodiments, the intracellular domain of an alpha chain of IL-2 receptor comprises or is a sequence that is at least 60% identical, at least 65% identical, at least 70% identical, at least 75% identical, at least 80% identical, at least 82% identical, at least 84% identical, at least 85%, at least 86% identical, at least 88% identical, at least 90% identical, at least 91% identical, at least 92% identical, at least 93% identical, at least 94% identical, at least 95% identical, at least 96% identical, at least 97% identical, at least 98% identical, at least 99% identical, or 100% identical to an intracellular domain of a wildtype alpha chain of IL-2 receptor (e.g., an intracellular domain of a wildtype human alpha chain of IL-2 receptor, e.g., SEQ ID NO: 6) (e.g., SEQ ID NO: 35, 36, or 37).

As one skilled in the art can appreciate, when amino acids that are not conserved between intracellular domains from wildtype alpha chains of IL-2 receptor protein from different species are mutated (e.g., substituted with a different amino acid) they are less likely to cause a decrease in the activity of the intracellular domain from an alpha chain of IL-2 receptor protein. In contrast, when amino acids that are conserved between the intracellular domains from wildtype alpha chains of IL-2 receptor protein from different species are mutated (e.g., substituted with a different amino acid) they are more likely to cause a decrease in the activity of the intracellular domain from an alpha chain of IL-2 receptor protein. In view of this knowledge, one skilled in the art can select which amino acid positions in an intracellular domain of an alpha chain of an IL-2 receptor protein (e.g., the non-conserved amino acids) can be substituted without decreasing the activity of the intracellular domain from the alpha chain of an IL-2 receptor protein.

In some embodiments, the intracellular domain of an alpha chain of an IL-2 receptor protein is a sequence of an intracellular domain of a wildtype alpha chain of IL-2 receptor protein (e.g., an intracellular domain of a wildtype human alpha chain of IL-2 receptor protein, e.g., SEQ ID NO: 6) (e.g., SEQ ID NO: 35, 36, or 37) having one to three (e.g., one, two, or three) amino acids removed from the N-terminus of the intracellular domain of the alpha chain of the IL-2 receptor protein. In some embodiments, the intracellular domain of an alpha chain of an IL-2 receptor protein is a sequence of an intracellular domain of a wildtype alpha chain of an IL-2 receptor protein (e.g., an intracellular domain of a wildtype human alpha chain of IL-2 receptor protein, e.g., SEQ ID NO: 5) (e.g., SEQ ID NO: 32, 33, or 34) having one to three (e.g., one, two, or three) amino acids removed from the C-terminus of the intracellular domain of the alpha chain of the IL-2 receptor protein. In some embodiments, the intracellular domain of an alpha chain of an IL-2 receptor protein is a sequence of an intracellular domain of a wildtype alpha chain of IL-2 receptor protein (e.g., an intracellular domain of a wildtype human alpha chain of IL-2 receptor protein, e.g., SEQ ID NO: 6) (e.g., SEQ ID NO: 35, 36, or 37) having one to three (e.g., one, two, or three) amino acids removed from the N-terminus of the intracellular domain of the alpha chain of the IL-2 receptor protein, and one to three (e.g., one, two, or three) amino acids removed from the C-terminus of the intracellular domain of the alpha chain of the IL-2 receptor protein.

Additional examples of intracellular domains from an alpha chain of an IL-2 receptor protein are known in the art.

### Linker Sequences

In some embodiments, the chimeric transmembrane protein comprises a linker sequence positioned between the extracellular IL-2 domain and the extracellular sushi domain. In some embodiments, the chimeric transmembrane protein comprises an additional linker sequence positioned between extracellular sushi domain and the transmembrane domain of an alpha chain of an IL-2 receptor. When the chimeric transmembrane protein comprises a linker sequence and an additional linker sequence, each the linker sequences can be the same or different from each other.

In some embodiments, the linker sequence or the additional linker sequence is 1 amino acid to about 50 amino acids, 1 amino acid to about 48 amino acids, 1 amino acid to about 46 amino acids, 1 amino acid to about 44 amino acids, 1 amino acid to about 42 amino acids, 1 amino acid to about 40 amino acids, 1 amino acid to about 38 amino acids, 1 amino acid to about 36 amino acids, 1 amino acid to about 34 amino acids, 1 amino acid to about 32 amino acids, 1 amino acid to about 30 amino acids, 1 amino acid to about 28 amino acids, 1 amino acid to about 26 amino acids, 1 amino acid to about 24 amino acids, 1 amino acid to about 22 amino acids, 1 amino acid to about 20 amino acids, 1 amino acid to about 18 amino acids, 1 amino acid to about 16 amino acids, 1 amino acid to about 14 amino acids, 1 amino acid to about 12 amino acids, 1 amino acid to about 10 amino acids, 1 amino acid to about 8 amino acids, 1 amino acid to about 6 amino acids, 1 amino acid to about 4 amino acids, 1 amino acid to about 3 amino acids, about 2 amino acids to about 50 amino acids, about 2 amino acids to about 48 amino acids, about 2 amino acids to about 46 amino acids, about 2 amino acids to about 44 amino acids, about 2 amino acids to about 42 amino acids, about 2 amino acids to about 40 amino acids, about 2 amino acids to about 38 amino acids, about 2 amino acids to about 36 amino acids, about 2 amino acids to about 34 amino acids, about 2 amino acids to about 32 amino acids, about 2 amino acids to about 30 amino acids, about 2 amino acids to about 28 amino acids, about 2 amino acids to about 26 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 4 amino acids, about 4 amino acids to about 50 amino acids, about 4 amino acids to about 48 amino acids, about 4 amino acids to about 46 amino acids, about 4 amino acids to about 44 amino acids, about 4 amino acids to about 42 amino acids, about 4 amino acids to about 40 amino acids, about 4 amino acids to about 38 amino acids, about 4 amino acids to about 36 amino acids, about 4 amino acids to about 34 amino acids, about 4 amino acids to about 32 amino acids, about 4 amino acids to about 30 amino acids, about 4 amino acids to about 28 amino acids, about 4 amino acids to about 26 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 6 amino acids to about 50 amino acids, about 6 amino acids to about 48 amino acids, about 6 amino acids to about 46 amino acids, about 6 amino acids to about 44 amino acids, about 6 amino acids to about 42 amino acids, about 6 amino acids to about 40 amino acids, about 6 amino acids to about 38 amino acids, about 6 amino acids to about 36 amino acids, about 6 amino acids to about 34 amino acids, about 6 amino acids to about 32 amino acids, about 6 amino acids to about 30 amino acids, about 6 amino acids to about 28 amino acids, about 6 amino acids to about 26 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 14 amino acids, about 6 amino acids to about 12 amino acids, about 6 amino acids to about 10 amino acids, about 6 amino acids to about 8 amino acids, about 8 amino acids to about 50 amino acids, about 8 amino acids to about 48 amino acids, about 8 amino acids to about 46 amino acids, about 8 amino acids to about 44 amino acids, about 8 amino acids to about 42 amino acids, about 8 amino acids to about 40 amino acids, about 8 amino acids to about 38 amino acids, about 8 amino acids to about 36 amino acids, about 8 amino acids to about 34 amino acids, about 8 amino acids to about 32 amino acids, about 8 amino acids to about 30 amino acids, about 8 amino acids to about 28 amino acids, about 8 amino acids to about 26 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 14 amino acids, about 8 amino acids to about 12 amino acids, about 8 amino acids to about 10 amino acids, about 10 amino acids to about 50 amino acids, about 10 amino acids to about 48 amino acids, about 10 amino acids to about 46 amino acids, about 10 amino acids to about 44 amino acids, about 10 amino acids to about 42 amino acids, about 10 amino acids to about 40 amino acids, about 10 amino acids to about 38 amino acids, about 10 amino acids to about 36 amino acids, about 10 amino acids to about 34 amino acids, about 10 amino acids to about 32 amino acids, about 10 amino acids to about 30 amino acids, about 10 amino acids to about 28 amino acids, about 10 amino acids to about 26 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 14 amino acids, about 10 amino acids to about 12 amino acids, about 12 amino acids to about 50 amino acids, about 12 amino acids to about 48 amino acids, about 12 amino acids to about 46 amino acids, about 12 amino acids to about 44 amino acids, about 12 amino acids to about 42 amino acids, about 12 amino acids to about 40 amino acids, about 12 amino acids to about 38 amino acids, about 12 amino acids to about 36 amino acids, about 12 amino acids to about 34 amino acids, about 12 amino acids to about 32 amino acids, about 12 amino acids to about 30 amino acids, about 12 amino acids to about 28 amino acids, about 12 amino acids to about 26 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 14 amino acids, about 14 amino acids to about 50 amino acids, about 14 amino acids to about 48 amino acids, about 14 amino acids to about 46 amino acids, about 14 amino acids to about 44 amino acids, about 14 amino acids to about 42 amino acids, about 14 amino acids to about 40 amino acids, about 14 amino acids to about 38 amino acids, about 14 amino acids to about 36 amino acids, about 14 amino acids to about 34 amino acids, about 14 amino acids to about 32 amino acids, about 14 amino acids to about 30 amino acids, about 14 amino acids to about 28 amino acids, about 14 amino acids to about 26 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 16 amino acids to about 50 amino acids, about 16 amino acids to about 48 amino acids, about 16 amino acids to about 46 amino acids, about 16 amino acids to about 44 amino acids, about 16 amino acids to about 42 amino acids, about 16 amino acids to about 40 amino acids, about 16 amino acids to about 38 amino acids, about 16 amino acids to about 36 amino acids, about 16 amino acids to about 34 amino acids, about 16 amino acids to about 32 amino acids, about 16 amino acids to about 30 amino acids, about 16 amino acids to about 28 amino acids, about 16 amino acids to about 26 amino acids, about 16 amino acids to about 24 amino acids, about 16 amino acids to about 22 amino acids, about 16 amino acids to about 20 amino acids, about 16 amino acids to about 18 amino acids, about 18 amino acids to about 50 amino acids, about 18 amino acids to about 48 amino acids, about 18 amino acids to about 46 amino acids, about 18 amino acids to about 44 amino acids, about 18 amino acids to about 42 amino acids, about 18 amino acids to about 40 amino acids, about 18 amino acids to about 38 amino acids, about 18 amino acids to about 36 amino acids, about 18 amino acids to about 34 amino acids, about 18 amino acids to about 32 amino acids, about 18 amino acids to about 30 amino acids, about 18 amino acids to about 28 amino acids, about 18 amino acids to about 26 amino acids, about 18 amino acids to about 24 amino acids, about 18 amino acids to about 22 amino acids, about 18 amino acids to about 20 amino acids, about 20 amino acids to about 50 amino acids, about 20 amino acids to about 48 amino acids, about 20 amino acids to about 46 amino acids, about 20 amino acids to about 44 amino acids, about 20 amino acids to about 42 amino acids, about 20 amino acids to about 40 amino acids, about 20 amino acids to about 38 amino acids, about 20 amino acids to about 36 amino acids, about 20 amino acids to about 34 amino acids, about 20 amino acids to about 32 amino acids, about 20 amino acids to about 30 amino acids, about 20 amino acids to about 28 amino acids, about 20 amino acids to about 26 amino acids, about 20 amino acids to about 24 amino acids, about 20 amino acids to about 22 amino acids, about 22 amino acids to about 50 amino acids, about 22 amino acids to about 48 amino acids, about 22 amino acids to about 46 amino acids, about 22 amino acids to about 44 amino acids, about 22 amino acids to about 42 amino acids, about 22 amino acids to about 40 amino acids, about 22 amino acids to about 38 amino acids, about 22 amino acids to about 36 amino acids, about 22 amino acids to about 34 amino acids, about 22 amino acids to about 32 amino acids, about 22 amino acids to about 30 amino acids, about 22 amino acids to about 28 amino acids, about 22 amino acids to about 26 amino acids, about 22 amino acids to about 24 amino acids, about 24 amino acids to about 50 amino acids, about 24 amino acids to about 48 amino acids, about 24 amino acids to about 46 amino acids, about 24 amino acids to about 44 amino acids, about 24 amino acids to about 42 amino acids, about 24 amino acids to about 40 amino acids, about 24 amino acids to about 38 amino acids, about 24 amino acids to about 36 amino acids, about 24 amino acids to about 34 amino acids, about 24 amino acids to about 32 amino acids, about 24 amino acids to about 30 amino acids, about 24 amino acids to about 28 amino acids, about 24 amino acids to about 26 amino acids, about 26 amino acids to about 50 amino acids, about 26 amino acids to about 48 amino acids, about 26 amino acids to about 46 amino acids, about 26 amino acids to about 44 amino acids, about 26 amino acids to about 42 amino acids, about 26 amino acids to about 40 amino acids, about 26 amino acids to about 38 amino acids, about 26 amino acids to about 36 amino acids, about 26 amino acids to about 34 amino acids, about 26 amino acids to about 32 amino acids, about 26 amino acids to about 30 amino acids, about 26 amino acids to about 28 amino acids, about 28 amino acids to about 50 amino acids, about 28 amino acids to about 48 amino acids, about 28 amino acids to about 46 amino acids, about 28 amino acids to about 44 amino acids, about 28 amino acids to about 42 amino acids, about 28 amino acids to about 40 amino acids, about 28 amino acids to about 38 amino acids, about 28 amino acids to about 36 amino acids, about 28 amino acids to about 34 amino acids, about 28 amino acids to about 32 amino acids, about 28 amino acids to about 30 amino acids, about 30 amino acids to about 50 amino acids, about 30 amino acids to about 48 amino acids, about 30 amino acids to about 46 amino acids, about 30 amino acids to about 44 amino acids, about 30 amino acids to about 42 amino acids, about 30 amino acids to about 40 amino acids, about 30 amino acids to about 38 amino acids, about 30 amino acids to about 36 amino acids, about 30 amino acids to about 34 amino acids, about 30 amino acids to about 32 amino acids, about 32 amino acids to about 50 amino acids, about 32 amino acids to about 48 amino acids, about 32 amino acids to about 46 amino acids, about 32 amino acids to about 44 amino acids, about 32 amino acids to about 42 amino acids, about 32 amino acids to about 40 amino acids, about 32 amino acids to about 38 amino acids, about 32 amino acids to about 36 amino acids, about 32 amino acids to about 34 amino acids, about 34 amino acids to about 50 amino acids, about 34 amino acids to about 48 amino acids, about 34 amino acids to about 46 amino acids, about 34 amino acids to about 44 amino acids, about 34 amino acids to about 42 amino acids, about 34 amino acids to about 40 amino acids, about 34 amino acids to about 38 amino acids, about 34 amino acids to about 36 amino acids, about 36 amino acids to about 50 amino acids, about 36 amino acids to about 48 amino acids, about 36 amino acids to about 46 amino acids, about 36 amino acids to about 44 amino acids, about 36 amino acids to about 42 amino acids, about 36 amino acids to about 40 amino acids, about 36 amino acids to about 38 amino acids, about 38 amino acids to about 50 amino acids, about 38 amino acids to about 48 amino acids, about 38 amino acids to about 46 amino acids, about 38 amino acids to about 44 amino acids, about 38 amino acids to about 42 amino acids, about 38 amino acids to about 40 amino acids, about 40 amino acids to about 50 amino acids, about 40 amino acids to about 48 amino acids, about 40 amino acids to about 46 amino acids, about 40 amino acids to about 44 amino acids, about 40 amino acids to about 42 amino acids, about 42 amino acids to about 50 amino acids, about 42 amino acids to about 48 amino acids, about 42 amino acids to about 46 amino acids, about 42 amino acids to about 44 amino acids, about 44 amino acids to about 50 amino acids, about 44 amino acids to about 48 amino acids, about 44 amino acids to about 46 amino acids, about 46 amino acids to about 50 amino acids, about 46 amino acids to about 48 amino acids, or about 48 amino acids to about 50 amino acids.

In some embodiments, the linker sequence and/or the additional linker sequence comprises a sequence of (SG)ₙ, where n can be 1, 2, 3, 4, 5, 6, 7, 8, 9, 10, 11, 12, 13, 14, 15, 16, 17, 18, 19, 20, 21, 22, 23, 24, or 25. In some embodiments, the linker sequence and/or the additional linker sequence comprises a sequence of (GS)ₙ, where n can be 1, 2, 3, 4, 5, 6, 7, 8, 9, 10, 11, 12, 13, 14, 15, 16, 17, 18, 19, 20, 21, 22, 23, 24, or 25.

In some embodiments, the linker sequence and/or the additional linker sequence comprises a sequence of (SGGS)ₙ, where n can be 1, 2, 3, 4, 5, 6, 7, 8, 9, 10, 11, 12, or 13. In some embodiments, the linker sequence and/or the additional linker sequence comprises a sequence of (SGGGS)ₙ, where n can be 1, 2, 3, 4, 5, 6, 7, 8, 9, or 10. In some embodiments, the linker sequence and/or the additional linker sequence comprises a sequence of (SGGGGS)ₙ, where n can be 1, 2, 3, 4, 5, 6, 7, 8, or 9.

In some embodiments, the linker sequence and/or the additional linker sequence can be or include the sequence of SGGGGSGGGGSGGGG (SEQ ID NO: 38). In some embodiments, the linker sequence and/or the additional linker sequence can be or include the sequence of ASTKGPSVFPLAPSSSGSG (SEQ ID NO: 39). In some embodiments, the linker sequence and/or the additional linker sequence can be or include the sequence of GGGGSGGGGSGGGGS (SEQ ID NO: 40). In some embodiments, the linker sequence and/or the additional linker sequence can be or include the sequence of GGGSGGGGSGGGGSGGGGSGGGS (SEQ ID NO: 2).

In some embodiments, the linker sequence and/or the additional linker sequence can be a Whitlow linker. In some embodiments, the Whitlow linker has the amino acid sequence of GSTSGSGKPGSGEGSTKG (SEQ ID NO: 41) or the nucleotide sequence encoding the Whitlow linker sequence is ggcagcaccagcggcagcggcaaaccgggcagcggcgaa ggcagcaccaaaggc (SEQ ID NO: 42).

In some embodiments, the linker sequence and/or the additional linker sequence can be a (G₄S)₅ linker. In some embodiments, the (G₄S)₅ linker has the amino acid sequence of GGGGSGGGGSGGGGSGGGGSGGGGS (SEQ ID NO: 43) or the nucleotide sequence encoding the (G4S) 5 linker sequence is ggcggtggtggttctggaggcggtgg cagcggtggaggtggctcaggaggaggaggtagcggcggcggagggagt (SEQ ID NO: 44).

In some embodiments, the linker sequence and/or the additional linker sequence can be or can include one or more of an IgG1, IgG2, IgG3, or IgG4 CH1, CH2, and CH3 domain. In some embodiments, the linker sequence and/or the additional linker sequence can be or can include CH2-CH3 human IgG1 domains. In some embodiments, the CH2-CH3 human IgG1 domains have a sequence of:

(SEQ ID NO: 45)
AEPKSPDKTHTCPPCPAPPVAGPSVFLFPPKPKDTLMIARTPEVTCWVDVS
HEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQG
NVFSCSVMHEALHNHYTQKSLSLSPGKKD.

In some embodiments, the linker sequence and/or the additional linker sequence can be or include a portion of the human CD8 extracellular sequence that is proximal to the human CD8 transmembrane domain. For example, the linker sequence and/or the additional linker sequence can be or include human CD8 sequence of:

(SEQ ID NO: 46)
TTTPAPRPPTPAPTIASQPLSLRPEACRPAAGGAVHTRGLDFACDI.

In some embodiments, the linker sequence and/or the additional linker sequence can be or include a human IgG1 hinge sequence. In some embodiments, the human IgG1 hinge sequence is AEPKSPDKTHTCPPCPKDPK (SEQ ID NO: 47).

In some embodiments, the linker sequence and/or the additional linker sequence has an alpha helix structure. In some embodiments, the linker sequence and/or the additional linker sequence is a coiled coil domain.

In some embodiments, the linker sequence and/or the additional linker sequence is a naturally-occurring amino acid sequence. In some embodiments, the linker sequence and/or the additional linker sequence is not a naturally-occurring amino acid sequence. In some embodiments, the linker sequence and/or the additional linker sequence comprises a sequence of SEQ ID NO: 2. In some embodiments, the linker sequence and/or the additional linker sequence consists of a sequence of SEQ ID NO: 2. Additional aspects and examples of linkers are known in the art.

### Chimeric Antigen Receptors

A chimeric antigen receptor (CAR) is a protein that includes an extracellular antigen-binding domain (e.g., any of the antigen-binding domains described herein or known in the art), a transmembrane domain (e.g., any of the transmembrane domains described herein or known in the art), a costimulatory domain (e.g., any of the costimulatory domains described herein or known in the art), and an immunoreceptor tyrosine-based activation motif (ITAM). Non-limiting aspects of chimeric antigen receptors are described in, e.g., Kershaw et al., Nature Reviews Immunol. 5(12):928-940, 2005; Eshhar et al., Proc. Natl. Acad. Sci. U.S.A. 90(2):720-724, 1993; Sadelain et al., Curr. Opin. Immunol. 21(2): 215-223, 2009; WO 2015/142675; WO 2015/150526; and WO 2014/134165, the disclosures of each of which are incorporated herein by reference in their entirety.

Some embodiments, a chimeric antigen receptors can include one or more (e.g., two, three, four, or five) costimulatory domain(s) (e.g., any combination of any of the exemplary costimulatory domains described herein or known in the art). Some embodiments of these chimeric antigen receptors include one or both of a 4-1BB costimulatory domain and a CD28 costimulatory domain.

Some embodiments, a chimeric antigen receptor can include one or more (e.g., two, three, four, or five) ITAMs (e.g., any of the ITAMs described herein or known in the art). In some embodiments of these chimeric antigen receptors, the ITAM includes a cytoplasmic signaling sequence from CD3ζ (e.g., human CD3ζ).

In some embodiments, one or more amino acids between the extracellular antigen-binding domain and the transmembrane domain is a sequence from the same endogenous single-chain polypeptide from which the transmembrane domain is derived. In some embodiments, one or more amino acids between the extracellular antigen-binding domain and the transmembrane domain is or includes a hinge region sequence of an antibody such as, without limitation, a human antibody (e.g., IgG1, IgG2, IgG3, or IgG4). In some embodiments, one or more amino acids between the extracellular antigen-binding domain and the transmembrane domain is or comprises a linker sequence (e.g., a non-naturally occurring linker sequence, e.g., GS or any of the other linker sequences described herein).

In some examples of any of the CARs described herein, going in the N-terminal to the C-terminal direction, the intracellular portion of the CAR includes a co-stimulatory domain and an intracellular signaling domain. In some examples of any of the CARs described herein, going in the N-terminal to the C-terminal direction, the intracellular portion of the CAR includes an intracellular signaling domain and a co-stimulatory domain.

In some examples of any of the CARs described herein, going in the C-terminal to the N-terminal direction, the intracellular portion of the CAR includes a co-stimulatory domain and an intracellular signaling domain. In some examples of any of the CARs described herein, going in the C-terminal to the N-terminal direction, the intracellular portion of the CAR includes an intracellular signaling domain and a co-stimulatory domain.

In some embodiments of any of the CARs described herein, the transmembrane is or includes a transmembrane domain of CD28, CD3 epsilon, CD4, CD5, CD6, CD8α, CD9, CD16, CD22, CD33, CD37, CD 45, CD64, CD80, CD86, CD134, 4-1BB, or CD154. Additional examples and aspects of transmembrane domains are described herein.

In some embodiments of any of the CARs described herein, the co-stimulatory domain is or includes the co-stimulatory domain of 4-1BB, CD28, CD2, CD4 or CD8. Additional examples and aspects of co-stimulatory domains are described herein.

A variety of methods that can be used to determine the K_{D} values of any of the CARs described herein are known in the art (e.g., an electrophoretic mobility shift assay, a filter binding assay, surface plasmon resonance, and a biomolecular binding kinetics assay, etc.).

Some embodiments of any of the chimeric antigen receptors described herein can further include a dimerization domain and/or a peptide tag.

### Antigen-Binding Domains

In some embodiments of the chimeric antigen receptors, the antigen-binding domain can be selected from a scFv, a scFv-Fc, a V_{H}H domain, a VNAR domain, a (scFv)₂, and a BiTE. Additional examples of antigen-binding domains that can be used the chimeric antigen receptors described herein are known in the art.

A single-chain Fv or scFv fragment includes a V_{H} domain and a V_{L} domain in a single polypeptide chain. The V_{H} and V_{L} are generally linked by a peptide linker. In other examples, the linker can be a single amino acid. In some examples, the linker can be a chemical bond. See, e.g., Pluckthun, Antibodies from E. coli. In Rosenberg M. & Moore G. P. (Eds.), The Pharmacology of Monoclonal Antibodies, Vol. 113, pp. 269-315, Spinger-Verlag, New York, 1994.

Sc-Fv-Fc fragments include an scFv attached to an Fc domain. For example, an Fc domain can be attached, e.g., to the C-terminus of the scFv. The Fc domain can follow the V_{L} or VII, depending on the orientation of the variable domains in the scFv. The Fc domain can be any Fc domain known in the art. In some examples, the Fc domain is an IgG1, IgG2, IgG3, or IgG4 Fc domain (e.g., a human IgG1, IgG2, IgG3, or IgG4 Fc domain).

BiTEs are antigen-binding domains that include two V_{L} and two V_{H} in a single polypeptide that together form two scFvs, which can each bind to different epitopes on the same antigen or each bind to different antigens. See, e.g., Baeuerle et al., Curr. Opin. Mol. Ther. 11:22-30, 2009; Wolf et al., Drug Discovery Today 10:1237-1244, 2005; and Huehls et al., Immunol. Cell Biol. 93:290-296, 2015.

A V_{H}H domain is a single monomeric variable antibody domain found in camelids, and a VNAR domain is a single monomeric variable antibody domain found in cartilaginous fish. V_{H}H domains and VNAR domains are described in, e.g., Van Audenhove et al., EBioMedicine 8:40-48, 2016; Krah et al., Immunopharmacol. Immunotoxicol. 38:21-28, 2016; Cromie et al., Curr. Top. Med. Chem. 15:2543-2557, 2016; Kijanka et al., Nanomedicine 10:161-174, 2015; Kovaleva et al., Expert. Opin. Biol. Ther. 14:1527-1539, 2014; De Meyer et al., Trends Biotechnol. 32:263-270, 2014; Mujic-Delic et al., Trends Pharmacol. Sci. 35:247-255, 2014; Muyldermans, Ann. Rev. Biochem. 82:775-797, 2013; Vincke et al., Methods Mol. Biol. 911:15-26, 2012;Rahbarizadeh et al., Immunol. Invest. 40:299-338, 2011; Van Bockstaele et al., Curr. Opin. Investig. Drugs 10:1212-1224, 2009; Wesolowski et al., Med. Microbiol. Immunol. 198:157-174, 2009; De Genst et al., Dev. Comp. Immunol. 30:187-198, 2006; Muyldermans, J. Biotechnol. 74:277-302, 2001; and Muyldermans et al., Trends Biochem. Sci. 26:230-235, 2001.

Any of the antigen-binding domains described herein can bind to an antigen with a dissociation equilibrium constant (K_{D}) of less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the antigen-binding protein complexes provided herein can bind to a first and/or second antigen with a K_{D} of about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰ M, or about 1×10⁻⁹ M to about 1×10⁻¹¹ M (inclusive). A variety of different methods known in the art can be used to determine the K_{D} value of an antigen-binding domain (e.g., an electrophoretic mobility shift assay, a filter binding assay, surface plasmon resonance, and a biomolecular binding kinetics assay, etc.).

### Antigens

In some embodiments, a chimeric antigen receptor described herein can bind to a single antigen (e.g., any of the exemplary antigens described herein or known in the art). In some embodiments, an antigen-binding domain described herein can bind to two or more different antigens (e.g., two or more of any of the exemplary antigens described herein or known in the art). Non-limiting examples of antigens include: glypican-3, HER2, A33 antigen, 9-0-acetyl-GD3, CA19-9 marker, BhCG, CA-125 marker, carboanhydrase IX (MN/CA IX), calreticulin, CCR5, CCR8, CD2, CD3, CD5, CD16, CD19, CD20, CD22, CD24, CD25, CD27, CD28, CD30, CD33, CD38, CD40L, CD44, CD44V6, CD63, CD70, CD84, CD96, CD100, CC123, CD133, CD137, CD138, CD150, CD152 (CTLA-4), CD160, CRTAM, CS1 (CD319), DNAM-1 (CD226), CD229, CD244, CD272 (BTLA), CD274 (PDL-1, B7H1), CD279 (PD-1), CD319, CD352, CRTAM (CD355), CD358, DR3, GITR (TNFRSF 18), HVEM, ICOS, LIGHT, LTBR, OX40, activating forms of KIR, NKG2C, NKG2D, NKG2E, one or more natural cytotoxicity receptors, NTB-A, PEN-5, carcinoma embryonic antigen (CEA; CD66e), desmoglein 4, E-cadherin neoepitope, endosialin, ephrin A2 (EphA2), epidermal growth factor receptor (EGFR), epithelial cell adhesion molecule (EpCAM), fucosyl GM1, GD2, GD3, GM2, ganglioside GM3, Globo H, glycoprotein 100, HER2/neu, HER3, HER4, insulin-like growth factor receptor 1, Lewis-Y, LG, Ly-6, melanoma-specific chondroitin-sulfate proteoglycan (MCSCP), mesothelin, MUC1, MUC2, MUC3, MUC4, MUC5AC, MUC5b, MUC7, MUC16, Mullerian inhibitory substance (MIS) receptor type II, plasma cell antigen, poly SA, PSCA, PSMA, sonic hedgehog (SHH), SAS, STEAP, sTn antigen, TNF-a precursor, 2B4 (CD244), β2-integrins, KIR, KIR2DL1, KIR2DL2, KIR2DL3, KIR3DL2, KIR-L, KLRGI, LAIR-1, NKG2A, NKR-P IA, Siglec-3, Siglec-7, Siglec-9, TCRa, TCRB, TCR5y, TIM1, LAG3, LAIR1, PD-1H, TIGIT, TIM2, and TIM3. Additional examples of antigens are known in the art.

### CAR Transmembrane Domains

In some embodiments, a chimeric antigen receptor includes a transmembrane domain, or portion thereof, from an endogenous polypeptide, where the endogenous polypeptide is selected from the group g of: an α chain of a T cell receptor, a β chain of the T cell receptor, a ζ chain of the T cell receptor, CD28 (also known as Tp44), CD3ε, CD3δ, CD3γ, CD33, CD37 (also known as GP52-40 or TSPAN26), CD64 (also known as FCGR1A), CD80 (also known as B7, B7-1, B7.1, BB1, CD28LG, CD28LG1, and LAB7), CD45 (also known as PTPRC, B220, CD45R, GP180, L-CA, LCA, LY5, T200, and protein tyrosine phosphatase, receptor type C), CD4, CD5 (also known as LEU1 and T1), CD8α (also known as Leu2, MAL, and p32), CD9 (also known as BTCC-1, DRAP-27, MIC3, MRP-1, TSPAN-29, and TSPAN29), CD16 (also known as FCGR3 and FCG3), CD22 (also known as SIGLEC-2 and SIGLEC2), CD86 (also known as B7-2, B7.2, B70, CD28LG2, and LAB72), CD134 (also known as TNFRSF4, ACT35, RP5-902P8.3, IMD16, OX40, TXGP1L, and tumor necrosis factor receptor superfamily member 4), CD137 (also known as TNFRSF9, 4-1BB, CDw137, ILA, and tumor necrosis factor receptor superfamily member 9), CD27 (also known as S152, S152.LPFS2, T14, TNFRSF7, and Tp55), CD152 (also known as CTLA4, ALPS5, CELIAC3, CTLA-4, GRD4, GSE, IDDM12, and cytotoxic T-lymphocyte associated protein 4), PD1 (also known as PDCD1, CD279, PD-1, SLEB2, hPD-1, hPD-1, hSLE1, and Programmed cell death 1), ICOS (also known as AILIM, CD278, and CVID1), CD272 (also known as BTLA and BTLA1), CD30 (also known as TNFRSF8, DIS166E, and Ki-1), GITR (also known as TNFRSF18, RP5-902P8.2, AITR, CD357, and GITR-D), HVEM (also known as TNFRSF14, RP3-395M20.6, ATAR, CD270, HVEA, HVEM, LIGHTR, and TR2), DAP10, and CD154 (also known as CD40LG, CD40L, HIGM1, IGM, IMD3, T-BAM, TNFSF5, TRAP, gp39, hCD40L, and CD40 ligand). The letters “CD” is the previous sentence stand for “Cluster of Differentiation.” E.g., CD3 stands for “Cluster of Differentiation 3.” In some embodiments, a chimeric antigen receptor includes a transmembrane domain, or portion thereof, from an endogenous mammalian (e.g., human) polypeptide (e.g., a mammalian or human homolog of any of the polypeptides listed above).

Any transmembrane domain, or portion thereof, that serves to anchor an endogenous polypeptide in a lipid bilayer (e.g., plasma membrane) of a mammalian cell is suitable for use in accordance with compositions and methods disclosed herein. In some embodiments, a chimeric antigen receptor includes a transmembrane domain, or portion thereof, from human CD28, e.g., Accession No. P01747, e.g., amino acids 153 to 179 of SEQ ID NO: 48. In some embodiments, a chimeric antigen receptor includes a transmembrane domain that is at least 80% (e.g., at least 82%, at least 84%, at least 85%, at least 86%, at least 88%, at least 90%, at least 92%, at least 94%, at least 95%, at least 96%, at least 98%, or at least 99% identical) to amino acids 153 to 179 of SEQ ID NO: 48, or a portion thereof.

SEQ ID NO: 48
MLRLLLALNLFPSIQVTGNKILVKQSPMLVAYDNAVNLSCKYSYNLFSREF
RASLHKGLDSAVEVCVVYGNYSQQLQVYSKTGFNCDGKLGNESVTFYLQNL
YVNQTDIYFCKIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRS

In some embodiments, a chimeric antigen receptor includes a transmembrane domain, or portion thereof, from human CD3, e.g., Accession No. P20963, e.g., amino acids 31 to 51 of SEQ ID NO: 49. In some embodiments, a chimeric antigen receptor includes a transmembrane domain that is or includes a sequence that is at least 80% (e.g., at least 82%, at least 84%, at least 85%, at least 86%, at least 88%, at least 90%, at least 92%, at least 94%, at least 95%, at least 96%, at least 98%, or at least 99% identical) to amino acids 31 to 51 of SEQ ID NO: 49.

SEQ ID NO: 49
MKWKALFTAAILQAQLPITEAQSFGLLDPKLCYLLDGILFIYGVILTALFL
RVKFSRSADAPAYQQGQNQLYNELNLGRREEYDVLDKRRGRDPEMGGKPQR
RKNPQEGLYNELQKDKMAEAYSEIGMKGERRRGKGHDGLYQGLSTATKDTY
DALHMQALPPR

In some embodiments, a chimeric antigen receptor includes a transmembrane domain, or portion thereof, of any one of SEQ ID Nos. 50-56.

(SEQ ID NO: 50)
LGLLVAGVLVLLVSLGVAIHLCC;
(SEQ ID NO: 51)
VAAILGLGLVLGLLGPLAILLALYLL;
(SEQ ID NO: 52)
ALIVLGGVAGLLLFIGLGIFFCVRC;
(SEQ ID NO: 53)
LCYLLDGILFIYGVILTALFLRV;
(SEQ ID NO: 54)
WVLVVVGGVLACYSLLVTVAFIIFWV;
(SEQ ID NO: 55)
IYIWAPLAGTCGVLLLSLVITLYC;
and
(SEQ ID NO: 56)
ALPAALAVISFLLGLGLGVACVLA.

In some embodiments, a chimeric antigen receptor includes a transmembrane domain that is or includes a sequence that is at least 80% (e.g., at least 82%, at least 84%, at least 85%, at least 86%, at least 88%, at least 90%, at least 92%, at least 94%, at least 95%, at least 96%, at least 98%, or at least 99% identical) to any one of SEQ ID Nos. 50-56.

As will be appreciated by those of ordinary skill in the art, certain endogenous polypeptides have two or more isoforms that differ at least in their primary polypeptide sequence. A chimeric antigen receptor disclosed herein can include a transmembrane domain that includes a sequence of amino acids from any isoform of an endogenous transmembrane protein (e.g., an endogenous mammalian, e.g., human, transmembrane protein) including, e.g., an isoform (e.g., a human isoform) of: an α chain of a T cell receptor, a β chain of the T cell receptor, a ζ chain of the T cell receptor, CD28, CD3ε, CD3δ, CD3γ, CD33, CD37, CD64, CD80, CD45, CD4, CD5, CD8α, CD9, CD16, CD22, CD86, CD134, CD137, CD27, CD152, PD1, or CD154.

In some embodiments, a transmembrane domain, or portion thereof, of a chimeric antigen receptor includes a sequence of amino acids that exhibits at least 85%, 86%, 87%, 88%, 89%, 90%, 91%, 92%, 93%, 94%, 95%, 96%, 97%, 98%, 99% or more sequence identity to the transmembrane domains from one or more of the following endogenous mammalian (e.g., human) transmembrane proteins: an α chain of a T cell receptor, a β chain of the T cell receptor, a ζ chain of the T cell receptor, CD28, CD3ε, CD3δ, CD3γ, CD33, CD37, CD64, CD80, CD45, CD4, CD5, CD8α, CD9, CD16, CD22, CD86, CD134, CD137, CD27, CD152, PD1, or CD154. In some embodiments, a transmembrane domain, or portion thereof, of a chimeric antigen receptor includes a sequence of amino acids having one or more amino acid substitutions, deletions, or additions as compared to the transmembrane domain of an endogenous mammalian (e.g., human) transmembrane protein: an α chain of a T cell receptor, a β chain of the T cell receptor, a ζ chain of the T cell receptor, CD28, CD3ε, CD3δ, CD3γ, CD33, CD37, CD64, CD80, CD45, CD4, CD5, CD8α, CD9, CD16, CD22, CD86, CD134, CD137, CD27, CD152, PD1, or CD154.

In some embodiments, a chimeric antigen receptor includes a synthetic transmembrane domain. In some cases, a synthetic transmembrane domain can include predominantly hydrophobic residues such as, without limitation, leucine and valine. In some embodiments, a synthetic transmembrane domain includes a triplet of phenylalanine, tryptophan, and valine at each end of the domain.

In some embodiments, a chimeric antigen receptor includes a transmembrane domain that is a chimeric transmembrane domain having portions of a transmembrane domain from two or more endogenous mammalian (e.g., human) transmembrane polypeptides such as, without limitation, an α chain of a T cell receptor, a β chain of the T cell receptor, a ζ chain of the T cell receptor, CD28, CD3ε, CD3δ, CD3γ, CD33, CD37, CD64, CD80, CD45, CD4, CD5, CD8α, CD9, CD16, CD22, CD86, CD134, CD137, CD27, CD152, PD1, and CD154, such that the two or more portions of transmembrane domains together constitute a functional transmembrane domain. In some embodiments, such a portion of a chimeric transmembrane domain can include one or more amino acid substitutions, deletions, or additions as compared to a corresponding portion of a wild type transmembrane domain.

A transmembrane domain can include one, two, three, four, five, six, seven, eight, nine, or ten contiguous amino acid sequences that each traverse a lipid bilayer when present in the corresponding endogenous polypeptide when expressed in a mammalian cell. As is known in the art, a transmembrane domain can, e.g., include at least one (e.g., two, three, four, five, six, seven, eight, nine, or ten) contiguous amino acid sequence (that traverses a lipid bilayer when present in the corresponding endogenous polypeptide when expressed in a mammalian cell) that has α-helical secondary structure in the lipid bilayer. In some embodiments, a transmembrane domain can include two or more contiguous amino acid sequences (that each traverse a lipid bilayer when present in the corresponding endogenous polypeptide when expressed in a mammalian cell) that form a β-barrel secondary structure in the lipid bilayer. Additional examples and features of transmembrane domains are known in the art.

### Costimulatory Domains

In normal lymphocytes, T cell activation is mediated by two classes of intracellular signaling domains. Primary signaling is initiated via MHC-mediated antigen-dependent activation via the T cell receptor (e.g., a TCR/CD3 complex). A secondary or costimulatory signal is provided by a different receptor that includes a costimulatory signaling domain, which acts in an antigen-independent manner. Signals generated through the signaling domain of the TCR alone are insufficient for complete T cell activation; a co-stimulatory signal is also required.

In some embodiments, a chimeric antigen receptor includes a costimulatory domain, or portion thereof, from an endogenous mammalian (e.g., human) transmembrane polypeptide selected from the group of: CD27 (also known as S152, S152.LPFS2, T14, TNFRSF7, and Tp55), CD28 (also known as Tp44), 4-1BB (also known as TNFRSF9, CD137, CDw137, ILA, and tumor necrosis factor receptor superfamily member 9), OX40 (also known as TNFRSF4, ACT35, RP5-902P8.3, IMD16, CD134, TXGP1L, and tumor necrosis factor receptor superfamily member 4), CD30 (also known as TNFRSF8, DIS166E, and Ki-1), CD40L (also known as CD40LG, CD154, HIGM1, IGM, IMD3, T-BAM, TNFSF5, TRAP, gp39, hCD40L, and CD40 ligand), CD40 (also known as Bp50, CDW40, TNFRSF5, p50, CD40 (protein), and CD40 molecule), PD-1 (also known as PDCD1, CD279, PD-1, SLEB2, hPD-1, hPD-1, hSLE1, and Programmed cell death 1), PD-LI (also known as CD274, B7-H, B7H1, PD-L1, PDCD1L1, PDCD1LG1, PDL1, CD274 molecule, and Programmed cell death 1 ligand 1), ICOS (also known as AILIM, CD278, and CVID1), LFA-1 (also known as Lymphocyte function-associated antigen 1), CD2 (also known as LFA-2, SRBC, T11, and CD2 molecule), CD7 (also known as GP40, LEU-9, TP41, Tp40, and CD7 molecule), CD160 (also known as BY55, NK1, NK28, and CD160 molecule), LIGHT (also known as TNFSF14, CD258, HVEML, LIGHT, LTg, TR2, THLG1D, and tumor necrosis factor superfamily member 14), BTLA (also known as CD272 and BTLA1), TIM3 (also known as HAVCR2, HAVcr-2, KIM-3, TIM3, TIMD-3, TIMD3, Tim-3, CD366, and hepatitis A virus cellular receptor 2), CD244 (also known as 2B4, NAIL, NKR2B4, Nmrk, SLAMF4, and CD244 molecule), CD80 (also known as B7, B7-1, B7.1, BB1, CD28LG, CD28LG1, LAB7, and CD80 molecule), LAG3 (also known as CD223 and lymphocyte activating 3), NKG2C (also known as CD314, D12S2489E, KLR, NKG2-D, NKG2D, and killer cell lectin like receptor K1), GITR (also known as TNFRSF18, RP5-902P8.2, AITR, CD357, and GITR-D), HVEM (also known as TNFRSF14, RP3-395M20.6, ATAR, CD270, HVEA, HVEM, LIGHTR, and TR2), TLR1, TLR2, TLR3, TLR4, TLR5, TLR6, TLR7, TLR8, TLR9, TLRIO, CARD11, CD54 (ICAM), CD83, DAP10, LAT, SLP76, TRIM, ZAP70, and B7-H3 (also known as CD276, 4Ig-B7-H3, B7H3, B7RP-2, and CD276 molecule). The letters “CD” is the previous sentence stand for “Cluster of Differentiation.” For example, CD3 stands for “Cluster of Differentiation 3.” In some embodiments, a single-chain chimeric polypeptide, a single-chain chimeric antigen receptor, or a multi-chain chimeric antigen receptor includes a costimulatory domain, or portion thereof, from an endogenous mammalian (e.g., human) transmembrane polypeptide (e.g., a mammalian or human homolog of any of the polypeptides listed above).

Any costimulatory domain, or portion thereof, that serves to provide a costimulatory signal is suitable for use in accordance with compositions and methods disclosed herein. In some embodiments, a chimeric antigen receptor includes a costimulatory domain, or portion thereof, from human CD28 (e.g. Accession No. P01747, e.g., from amino acids 180 to 220 of SEQ ID NO: 57). In some embodiments, a costimulatory domain is or includes a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical (or is identical) to amino acids 180 to 220 of SEQ ID NO: 57, or a fragment thereof.

SEQ ID NO: 57
MLRLLLALNLFPSIQVTGNKILVKQSPMLVAYDNAVNLSCKYSYNLFSREF
RASLHKGLDSAVEVCVVYGNYSQQLQVYSKTGFNCDGKLGNEVTFYLQNLY
VNQTDIYFCKIEVMYPPPYLDNEKSNGTIIHVKGKHLCPSPLFPGPSKPFW
VLVVVGGVLACYSLLVTVAFIIFWVRSKRSRLLHSDYMNMTPRRPGPTRKH
YQPYAPPRDFAAYRS

In some embodiments, a chimeric antigen receptor includes a costimulatory domain, or portion thereof, from human 4-1BB (e.g. Accession No. Q07011, e.g., from amino acids 214 to 255 of SEQ ID NO: 58). In some embodiments, a costimulatory domain is or includes a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to amino acids 214 to 255 of SEQ ID NO: 58, or a portion thereof.

SEQ ID NO: 58
MGNSCYNIVATLLLVLNFERTRSLQDPCSNCPAGTFCDNNRNQICSPCPPN
SFSSAGGQRTCDICRQCKGVFRTRKECSSTSNAECDCTPGFHCLGAGCSMC
EQDCKQGQELTKKGCKDCCFGTFNDQKRGICRPWTNCSLDGKSVLVNGTKE
RDVVCGPSPADLSPGASSVTPPAPAREPGHSPQIISFFLALTSTALLFLLF
FLTLRFSVVKRGRKKLLYIFKQPFMRPVQTTQEEDGCSCRFPEEEEGGCEL

A chimeric antigen receptor disclosed herein can include a costimulatory domain that includes a sequence of amino acids from any isoform of an endogenous mammalian (e.g., human) transmembrane polypeptide having a costimulatory domain including, e.g., an isoform of: CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, or B7-H3 (including, without limitation, a mammalian or human homolog of any of these polypeptides).

In some embodiments, a costimulatory domain, or portion thereof, of a chimeric antigen receptor includes a sequence of amino acids that exhibits at least 85%, 86%, 87%, 88%, 89%, 90%, 91%, 92%, 93%, 94%, 95%, 96%, 97%, 98%, 99% or more sequence identity to a costimulatory domain from one or more of a mammalian (e.g., human) CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, or B7-H3. In some embodiments, a costimulatory domain, or portion thereof, of a chimeric antigen receptor includes a sequence of amino acids having one or more amino acid substitutions, deletions, or additions as compared to a costimulatory domain of one or more of an endogenous mammalian (e.g., human) transmembrane polypeptide: an α chain of a T cell receptor, a β chain of the T cell receptor, a ζ chain of the T cell receptor, CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, or B7-H3 (including, without limitation, a mammalian or human homolog of any of these polypeptides).

In some embodiments, a chimeric antigen receptor includes a costimulatory domain that is a chimeric costimulatory domain having portions of a costimulatory domain from two or more endogenous mammalian (e.g., human) transmembrane polypeptides including, without limitation, CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, or B7-H3 (including, without limitation, a mammalian or human homolog of any of these polypeptides), such that the two or more portions of the transmembrane domains together constitute a functional costimulatory domain. In some embodiments, such a portion of a chimeric costimulatory domain can include one or more amino acid substitutions, deletions, or additions as compared to a corresponding portion of a wildtype costimulatory domain.

A costimulatory domain of a chimeric antigen receptor disclosed herein can be of any suitable length. For example, a costimulatory domain can have a length of about 20 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, or about 25 amino acids (inclusive); about 25 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, or about 30 amino acids (inclusive); about 30 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, or about 35 amino acids (inclusive); about 35 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, or about 40 amino acids (inclusive); about 40 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, or about 45 amino acids (inclusive); about 45 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, or about 50 amino acids (inclusive); about 50 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, or about 55 amino acids (inclusive); about 55 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, or about 60 amino acids (inclusive); about 60 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, or about 65 amino acids (inclusive); about 65 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, or about 70 amino acids (inclusive); about 70 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, or about 80 amino acids (inclusive); about 80 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, or about 90 amino acids (inclusive); about 90 amino acids to about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, or about 100 amino acids (inclusive); about 100 amino acids to about 200 amino acids, about 190 amino acids, about 180 amino acids, about 170 amino acids, about 160 amino acids, about 150 amino acids, about 140 amino acids, about 130 amino acids, about 120 amino acids, or about 110 amino acids (inclusive); about 110 amino acids to about 200 amino acids, about 190 amino acids, about 180 amino acids, about 170 amino acids, about 160 amino acids, about 150 amino acids, about 140 amino acids, about 130 amino acids, or about 120 amino acids (inclusive); about 120 amino acids to about 200 amino acids, about 190 amino acids, about 180 amino acids, about 170 amino acids, about 160 amino acids, about 150 amino acids, about 140 amino acids, or about 130 amino acids (inclusive); about 130 amino acids to about 200 amino acids, about 190 amino acids, about 180 amino acids, about 170 amino acids, about 160 amino acids, about 150 amino acids, or about 140 amino acids (inclusive); about 140 amino acids to about 200 amino acids, about 190 amino acids, about 180 amino acids, about 170 amino acids, about 160 amino acids, or about 150 amino acids (inclusive); about 150 amino acids to about 200 amino acids, about 190 amino acids, about 180 amino acids, about 170 amino acids, or about 160 amino acids (inclusive); about 160 amino acids to about 200 amino acids, about 190 amino acids, about 180 amino acids, or about 170 amino acids (inclusive); about 170 amino acids to about 200 amino acids, about 190 amino acids, or about 180 amino acids (inclusive); about 180 amino acids to about 200 amino acids or about 190 amino acids (inclusive); or about 190 amino acids to about 200 amino acids (inclusive).

In some embodiments, a chimeric antigen receptor includes two or more costimulatory domains, e.g., two, three, four, or five, or more costimulatory domains. In some embodiments, the two or more costimulatory domains are identical (e.g., they have the same amino acid sequence). In some embodiments, the costimulatory domains are not identical. For example, the costimulatory domains can be selected from different endogenous mammalian (e.g., human) transmembrane polypeptides including, without limitation, CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, or B7-H3 (including, without limitation, a mammalian or human homolog of any of these polypeptides). In some embodiments, the two or more costimulatory domains can differ from each other by one or more (e.g., two, three, four, or five) amino acid substitutions, deletions, or additions. In some embodiments, the two or more costimulatory domains exhibit at least 85%, 86%, 87%, 88%, 89%, 90%, 91%, 92%, 93%, 94%, 95%, 96%, 97%, 98%, 99% or more sequence identity to each other.

### Immunoreceptor Tyrosine-Based Activation Motifs (ITAMs)

ITAMs include a tyrosine separated from a leucine or isoleucine by any two other amino acids, and can thus be represented as, e.g., Tyr-X-X-Leu/Ile. ITAMs are typically repeated (e.g., two or more times) in the cytoplasmic tails of certain cell surface proteins of the immune system, and are typically separated by between six and eight amino acids.

In some embodiments, a chimeric antigen receptor includes an ITAM, or portion thereof, from an endogenous mammalian (e.g., human) polypeptide, wherein endogenous mammalian (e.g., human) polypeptide is selected from the group of: CD3ζ (also referred to as CD3 zeta), CD3—(CD3 delta), CD3ε (CD3 epsilon), CD3γ (CD3 gamma), DAP12, FCεR1γ (Fc epsilon receptor I gamma chain), FcRy, FcRft, CD35, CD22, CD79A (antigen receptor complex-associated protein alpha chain), CD79B (antigen receptor complex-associated protein beta chain), and CD66d. The letters “CD” is the previous sentence stand for “Cluster of Differentiation.” For example, CD3 stands for “Cluster of Differentiation 3.”

Any ITAM, or portion thereof, that serves to mediate signaling in an endogenous mammalian (e.g., human) transmembrane protein suitable for use in accordance with compositions and methods disclosed herein. In some embodiments, a chimeric antigen receptor includes an ITAM, or portion thereof, from human CD3 zeta (e.g. Accession No. P20963, e.g., an ITAM present in amino acids 52-164 of SEQ ID NO: 59, or a portion thereof; or SEQ ID NO: 60 or a portion thereof). In some embodiments, an ITAM comprises a sequence that is at least 80% (e.g., at least 82%, at least 84%, at least 86%, at least 88%, at least 90%, at least 92%, at least 94%, at least 96%, at least 98%, at least 99%, or 100% identical to: the sequence of amino acids 52-165 of SEQ ID NO: 59 (or a portion thereof), or the sequence of SEQ ID NO: 60 (or a portion thereof).

SEQ ID NO: 59
MKWKALFTAAILQAQLPITEAQSFGLLDPKLCYLLDGILFIYGVILTALFL
RVKFSRSADAPAYQQGQNQLYNELNLGRREEYDVLDKRRGRDPEMGGKPQR
RKNPQEGLYNELQKDKMAEAYSEIGMKGERRRGKGHDGLYQGLSTATKDTY
DALHMQALPPR
(Human CD3 zeta signaling domain)
SEQ ID NO: 60
LRVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDTY
DALHMQALPPR
(cDNA encoding human CD3 zeta signaling domain of
SEQ ID NO: 60)
SEQ ID NO: 61
ctgagagtgaagttcagcaggagcgcagacgcccccgcgtaccagcagggc
cagaaccagctctataacgagctcaatctaggacgaagagaggagtacgat
gttttggacaagagacgtggccgggaccctgagatggggggaaagccgaga
aggaagaaccctcaggaaggcctgtacaatgaactgcagaaagataagatg
gcggaggcctacagtgagattgggatgaaaggcgagcgccggaggggcaag
gggcacgatggcctttaccagggtctcagtacagccaccaaggacacctac
gacgcccttcacatgcaggccctgccccctcgc

As will be appreciated by those of ordinary skill in the art, certain polypeptides have two or more isoforms that differ at least in their primary polypeptide sequence. For example, different isoforms can be generated as a result of alternative splicing. A chimeric antigen receptor disclosed herein can include an ITAM that includes a sequence of amino acids from any isoform of an endogenous mammalian transmembrane polypeptide having an ITAM including, e.g., a mammalian (e.g., human) isoform of: CD3ζ, CD3D, CD3E, CD3G, DAP12, FCERIG, FcRy, FcRft, CD35, CD22, CD79A, CD79B, or CD66d.

In some embodiments, an ITAM, or portion thereof, of a chimeric antigen receptor includes a sequence of amino acids having one or more (e.g., two, three, four, or five) amino acid substitutions, deletions, or additions as compared to an ITAM of one or more of an ITAM in an endogenous mammalian (e.g., human) transmembrane protein, such as, CD3ζ, CD3D, CD3E, CD3G, DAP12, FCER1G, FcRy, FcRft, CD35, CD22, CD79A, CD79B, or CD66d. For example, the tyrosine and leucine or isoleucine of an ITAM could be retained, while the two amino acids separating them could be replaced with different amino acids.

In some embodiments, a chimeric antigen receptor includes an ITAM that is a chimeric ITAM having portions of an ITAM from two or more endogenous mammalian (e.g., human) transmembrane polypeptides including, without limitation, CD3ζ, CD3D, CD3E, CD3G, DAP12, FCER1G, FcRy, FcRft, CD35, CD22, CD79A, CD79B, or CD66d (including, without limitation, a mammalian or human homolog of any of these polypeptides), such that the two or more ITAM portions together constitute a functional ITAM. In some embodiments, such a portion of a chimeric ITAM can include one or more amino acid substitutions, deletions, or additions as compared to a corresponding portion of a wild type ITAM.

In some embodiments, a chimeric antigen receptor includes two or more ITAMs, e.g., two, three, four, or five, or more ITAMs. In some embodiments, the two or more ITAMs are identical (e.g., they have the same amino acid sequence). In some embodiments, the two or more ITAMs are not identical. For example, the ITAMs can be selected from different endogenous mammalian (e.g., human) transmembrane polypeptides including, without limitation, CD3ζ, CD3D, CD3E, CD3G, DAP12, FCER1G, FcRy, FcRft, CD35, CD22, CD79A, CD79B (including, without limitation, a mammalian or human homolog of any of these polypeptides). In some embodiments, the two or more ITAMs can differ from each other by one or more amino acid substitutions, deletions, or additions.

### CAR-Linker Sequences

Any two neighboring domains of a chimeric antigen receptor can be separated by a linker sequence (e.g., any of the exemplary linker sequences described herein or known in the art).

In some embodiments, the linker sequence between the antigen-binding domain and the transmembrane domain can be 1 amino acid to about 250 amino acids, 1 amino acid to about 240 amino acids, 1 amino acid to about 230 amino acids, 1 amino acid to about 220 amino acids, 1 amino acid to about 210 amino acids, 1 amino acid to about 200 amino acids, 1 amino acid to about 190 amino acids, 1 amino acid to about 180 amino acids, 1 amino acid to about 170 amino acids, 1 amino acid to about 160 amino acids, 1 amino acid to about 150 amino acids, 1 amino acid to about 140 amino acids, 1 amino acid to about 130 amino acids, 1 amino acid to about 120 amino acids, 1 amino acid to about 110 amino acids, 1 amino acid to about 100 amino acids, 1 amino acid to about 95 amino acids, 1 amino acid to about 90 amino acids, 1 amino acid to about 85 amino acids, 1 amino acid to about 80 amino acids, 1 amino acid to about 75 amino acids, 1 amino acid to about 70 amino acids, 1 amino acid to about 65 amino acids, 1 amino acid to about 60 amino acids, 1 amino acid to about 55 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 5 amino acids, about 5 amino acids to about 250 amino acids, about 5 amino acids to about 240 amino acids, about 5 amino acids to about 230 amino acids, about 5 amino acids to about 220 amino acids, about 5 amino acids to about 210 amino acids, about 5 amino acids to about 200 amino acids, about 5 amino acids to about 190 amino acids, about 5 amino acids to about 180 amino acids, about 5 amino acids to about 170 amino acids, about 5 amino acids to about 160 amino acids, about 5 amino acids to about 150 amino acids, about 5 amino acids to about 140 amino acids, about 5 amino acids to about 130 amino acids, about 5 amino acids to about 120 amino acids, about 5 amino acids to about 110 amino acids, about 5 amino acids to about 100 amino acids, about 5 amino acids to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 250 amino acids, about 10 amino acids to about 240 amino acids, about 10 amino acids to about 230 amino acids, about 10 amino acids to about 220 amino acids, about 10 amino acids to about 210 amino acids, about 10 amino acids to about 200 amino acids, about 10 amino acids to about 190 amino acids, about 10 amino acids to about 180 amino acids, about 10 amino acids to about 170 amino acids, about 10 amino acids to about 160 amino acids, about 10 amino acids to about 150 amino acids, about 10 amino acids to about 140 amino acids, about 10 amino acids to about 130 amino acids, about 10 amino acids to about 120 amino acids, about 10 amino acids to about 110 amino acids, about 10 amino acids to about 100 amino acids, about 10 amino acids to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 250 amino acids, about 15 amino acids to about 240 amino acids, about 15 amino acids to about 230 amino acids, about 15 amino acids to about 220 amino acids, about 15 amino acids to about 210 amino acids, about 15 amino acids to about 200 amino acids, about 15 amino acids to about 190 amino acids, about 15 amino acids to about 180 amino acids, about 15 amino acids to about 170 amino acids, about 15 amino acids to about 160 amino acids, about 15 amino acids to about 150 amino acids, about 15 amino acids to about 140 amino acids, about 15 amino acids to about 130 amino acids, about 15 amino acids to about 120 amino acids, about 15 amino acids to about 110 amino acids, about 15 amino acids to about 100 amino acids, about 15 amino acids to about 95 amino acids, about 15 amino acids to about 90 amino acids, about 15 amino acids to about 85 amino acids, about 15 amino acids to about 80 amino acids, about 15 amino acids to about 75 amino acids, about 15 amino acids to about 70 amino acids, about 15 amino acids to about 65 amino acids, about 15 amino acids to about 60 amino acids, about 15 amino acids to about 55 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 250 amino acids, about 20 amino acids to about 240 amino acids, about 20 amino acids to about 230 amino acids, about 20 amino acids to 220 amino acids, about 20 amino acids to about 210 amino acids, about 20 amino acids to about 200 amino acids, about 20 amino acids to about 190 amino acids, about 20 amino acids to about 180 amino acids, about 20 amino acids to about 170 amino acids, about 20 amino acids to about 160 amino acids, about 20 amino acids to about 150 amino acids, about 20 amino acids to about 140 amino acids, about 20 amino acids to about 130 amino acids, about 20 amino acids to about 120 amino acids, about 20 amino acids to about 110 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 250 amino acids, about 25 amino acids to about 240 amino acids, about 25 amino acids to about 230 amino acids, about 25 amino acids to about 220 amino acids, about 25 amino acids to about 210 amino acids, about 25 amino acids to about 200 amino acids, about 25 amino acids to about 190 amino acids, about 25 amino acids to about 180 amino acids, about 25 amino acids to about 170 amino acids, about 25 amino acids to about 160 amino acids, about 25 amino acids to about 150 amino acids, about 25 amino acids to about 140 amino acids, about 25 amino acids to about 130 amino acids, about 25 amino acids to about 120 amino acids, about 25 amino acids to about 110 amino acids, about 25 amino acids to about 100 amino acids, about 25 amino acids to about 95 amino acids, about 25 amino acids to about 90 amino acids, about 25 amino acids to about 85 amino acids, about 25 amino acids to about 80 amino acids, about 25 amino acids to about 75 amino acids, about 25 amino acids to about 70 amino acids, about 25 amino acids to about 65 amino acids, about 25 amino acids to about 60 amino acids, about 25 amino acids to about 55 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 250 amino acids, about 30 amino acids to about 240 amino acids, about 30 amino acids to about 230 amino acids, about 30 amino acids to about 220 amino acids, about 30 amino acids to about 210 amino acids, about 30 amino acids to about 200 amino acids, about 30 amino acids to about 190 amino acids, about 30 amino acids to about 180 amino acids, about 30 amino acids to about 170 amino acids, about 30 amino acids to about 160 amino acids, about 30 amino acids to about 150 amino acids, about 30 amino acids to about 140 amino acids, about 30 amino acids to about 130 amino acids, about 30 amino acids to about 120 amino acids, about 30 amino acids to about 110 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 35 amino acids to about 250 amino acids, about 35 amino acids to about 240 amino acids, about 35 amino acids to about 230 amino acids, about 35 amino acids to about 220 amino acids, about 35 amino acids to about 210 amino acids, about 35 amino acids to about 200 amino acids, about 35 amino acids to about 190 amino acids, about 35 amino acids to about 180 amino acids, about 35 amino acids to about 170 amino acids, about 35 amino acids to about 160 amino acids, about 35 amino acids to about 150 amino acids, about 35 amino acids to about 140 amino acids, about 35 amino acids to about 130 amino acids, about 35 amino acids to about 120 amino acids, about 35 amino acids to about 110 amino acids, about 35 amino acids to about 100 amino acids, about 35 amino acids to about 95 amino acids, about 35 amino acids to about 90 amino acids, about 35 amino acids to about 85 amino acids, about 35 amino acids to about 80 amino acids, about 35 amino acids to about 75 amino acids, about 35 amino acids to about 70 amino acids, about 35 amino acids to about 65 amino acids, about 35 amino acids to about 60 amino acids, about 35 amino acids to about 55 amino acids, about 35 amino acids to about 50 amino acids, about 35 amino acids to about 45 amino acids, about 35 amino acids to about 40 amino acids, about 40 amino acids to about 250 amino acids, about 40 amino acids to about 240 amino acids, about 40 amino acids to about 230 amino acids, about 40 amino acids to about 220 amino acids, about 40 amino acids to about 210 amino acids, about 40 amino acids to about 200 amino acids, about 40 amino acids to about 190 amino acids, about 40 amino acids to about 180 amino acids, about 40 amino acids to about 170 amino acids, about 40 amino acids to about 160 amino acids, about 40 amino acids to about 150 amino acids, about 40 amino acids to about 140 amino acids, about 40 amino acids to about 130 amino acids, about 40 amino acids to about 120 amino acids, about 40 amino acids to about 110 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 45 amino acids to about 250 amino acids, about 45 amino acids to about 240 amino acids, about 45 amino acids to about 230 amino acids, about 45 amino acids to about 220 amino acids, about 45 amino acids to about 210 amino acids, about 45 amino acids to about 200 amino acids, about 45 amino acids to about 190 amino acids, about 45 amino acids to about 180 amino acids, about 45 amino acids to about 170 amino acids, about 45 amino acids to about 160 amino acids, about 45 amino acids to about 150 amino acids, about 45 amino acids to about 140 amino acids, about 45 amino acids to about 130 amino acids, about 45 amino acids to about 120 amino acids, about 45 amino acids to about 110 amino acids, about 45 amino acids to about 100 amino acids, about 45 amino acids to about 95 amino acids, about 45 amino acids to about 90 amino acids, about 45 amino acids to about 85 amino acids, about 45 amino acids to about 80 amino acids, about 45 amino acids to about 75 amino acids, about 45 amino acids to about 70 amino acids, about 45 amino acids to about 65 amino acids, about 45 amino acids to about 60 amino acids, about 45 amino acids to about 55 amino acids, about 45 amino acids to about 50 amino acids, about 50 amino acids to about 250 amino acids, about 50 amino acids to about 240 amino acids, about 50 amino acids to about 230 amino acids, about 50 amino acids to about 220 amino acids, about 50 amino acids to about 210 amino acids, about 50 amino acids to about 200 amino acids, about 50 amino acids to about 190 amino acids, about 50 amino acids to about 180 amino acids, about 50 amino acids to about 170 amino acids, about 50 amino acids to about 160 amino acids, about 50 amino acids to about 150 amino acids, about 50 amino acids to about 140 amino acids, about 50 amino acids to about 130 amino acids, about 50 amino acids to about 120 amino acids, about 50 amino acids to about 110 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 55 amino acids to about 250 amino acids, about 55 amino acids to about 240 amino acids, about 55 amino acids to about 230 amino acids, about 55 amino acids to about 220 amino acids, about 55 amino acids to about 210 amino acids, about 55 amino acids to about 200 amino acids, about 55 amino acids to about 190 amino acids, about 55 amino acids to about 180 amino acids, about 55 amino acids to about 170 amino acids, about 55 amino acids to about 160 amino acids, about 55 amino acids to about 150 amino acids, about 55 amino acids to about 140 amino acids, about 55 amino acids to about 130 amino acids, about 55 amino acids to about 120 amino acids, about 55 amino acids to about 110 amino acids, about 55 amino acids to about 100 amino acids, about 55 amino acids to about 95 amino acids, about 55 amino acids to about 90 amino acids, about 55 amino acids to about 85 amino acids, about 55 amino acids to about 80 amino acids, about 55 amino acids to about 75 amino acids, about 55 amino acids to about 70 amino acids, about 55 amino acids to about 65 amino acids, about 55 amino acids to about 60 amino acids, about 60 amino acids to about 250 amino acids, about 60 amino acids to about 240 amino acids, about 60 amino acids to about 230 amino acids, about 60 amino acids to about 220 amino acids, about 60 amino acids to about 210 amino acids, about 60 amino acids to about 200 amino acids, about 60 amino acids to about 190 amino acids, about 60 amino acids to about 180 amino acids, about 60 amino acids to about 170 amino acids, about 60 amino acids to about 160 amino acids, about 60 amino acids to about 150 amino acids, about 60 amino acids to about 140 amino acids, about 60 amino acids to about 130 amino acids, about 60 amino acids to about 120 amino acids, about 60 amino acids to about 110 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 65 amino acids to about 250 amino acids, about 65 amino acids to about 240 amino acids, about 65 amino acids to about 230 amino acids, about 65 amino acids to about 220 amino acids, about 65 amino acids to about 210 amino acids, about 65 amino acids to about 200 amino acids, about 65 amino acids to about 190 amino acids, about 65 amino acids to about 180 amino acids, about 65 amino acids to about 170 amino acids, about 65 amino acids to about 160 amino acids, about 65 amino acids to about 150 amino acids, about 65 amino acids to about 140 amino acids, about 65 amino acids to about 130 amino acids, about 65 amino acids to about 120 amino acids, about 65 amino acids to about 110 amino acids, about 65 amino acids to about 100 amino acids, about 65 amino acids to about 95 amino acids, about 65 amino acids to about 90 amino acids, about 65 amino acids to about 85 amino acids, about 65 amino acids to about 80 amino acids, about 65 amino acids to about 75 amino acids, about 65 amino acids to about 70 amino acids, about 70 amino acids to about 250 amino acids, about 70 amino acids to about 240 amino acids, about 70 amino acids to about 230 amino acids, about 70 amino acids to about 220 amino acids, about 70 amino acids to about 210 amino acids, about 70 amino acids to about 200 amino acids, about 70 amino acids to about 190 amino acids, about 70 amino acids to about 180 amino acids, about 70 amino acids to about 170 amino acids, about 70 amino acids to about 160 amino acids, about 70 amino acids to about 150 amino acids, about 70 amino acids to about 140 amino acids, about 70 amino acids to about 130 amino acids, about 70 amino acids to about 120 amino acids, about 70 amino acids to about 110 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 70 amino acids to about 75 amino acids, about 75 amino acids to about 250 amino acids, about 75 amino acids to about 240 amino acids, about 75 amino acids to about 230 amino acids, about 75 amino acids to about 220 amino acids, about 75 amino acids to about 210 amino acids, about 75 amino acids to about 200 amino acids, about 75 amino acids to about 190 amino acids, about 75 amino acids to about 180 amino acids, about 75 amino acids to about 170 amino acids, about 75 amino acids to about 160 amino acids, about 75 amino acids to about 150 amino acids, about 75 amino acids to about 140 amino acids, about 75 amino acids to about 130 amino acids, about 75 amino acids to about 120 amino acids, about 75 amino acids to about 110 amino acids, about 75 amino acids to about 100 amino acids, about 75 amino acids to about 95 amino acids, about 75 amino acids to about 90 amino acids, about 75 amino acids to about 85 amino acids, about 75 amino acids to about 80 amino acids, about 80 amino acids to about 250 amino acids, about 80 amino acids to about 240 amino acids, about 80 amino acids to about 230 amino acids, about 80 amino acids to about 220 amino acids, about 80 amino acids to about 210 amino acids, about 80 amino acids to about 200 amino acids, about 80 amino acids to about 190 amino acids, about 80 amino acids to about 180 amino acids, about 80 amino acids to about 170 amino acids, about 80 amino acids to about 160 amino acids, about 80 amino acids to about 150 amino acids, about 80 amino acids to about 140 amino acids, about 80 amino acids to about 130 amino acids, about 80 amino acids to about 120 amino acids, about 80 amino acids to about 110 amino acids, about 80 amino acids to about 100 amino acids, about 80 amino acids to about 95 amino acids, about 80 amino acids to about 90 amino acids, about 80 amino acids to about 85 amino acids, about 85 amino acids to about 250 amino acids, about 85 amino acids to about 240 amino acids, about 85 amino acids to about 230 amino acids, about 85 amino acids to about 220 amino acids, about 85 amino acids to about 210 amino acids, about 85 amino acids to about 200 amino acids, about 85 amino acids to about 190 amino acids, about 85 amino acids to about 180 amino acids, about 85 amino acids to about 170 amino acids, about 85 amino acids to about 160 amino acids, about 85 amino acids to about 150 amino acids, about 85 amino acids to about 140 amino acids, about 85 amino acids to about 130 amino acids, about 85 amino acids to about 120 amino acids, about 85 amino acids to about 110 amino acids, about 85 amino acids to about 100 amino acids, about 85 amino acids to about 95 amino acids, about 85 amino acids to about 90 amino acids, about 90 amino acids to about 250 amino acids, about 90 amino acids to about 240 amino acids, about 90 amino acids to about 230 amino acids, about 90 amino acids to about 220 amino acids, about 90 amino acids to about 210 amino acids, about 90 amino acids to about 200 amino acids, about 90 amino acids to about 190 amino acids, about 90 amino acids to about 180 amino acids, about 90 amino acids to about 170 amino acids, about 90 amino acids to about 160 amino acids, about 90 amino acids to about 150 amino acids, about 90 amino acids to about 140 amino acids, about 90 amino acids to about 130 amino acids, about 90 amino acids to about 120 amino acids, about 90 amino acids to about 110 amino acids, about 90 amino acids to about 100 amino acids, about 90 amino acids to about 95 amino acids, about 95 amino acids to about 250 amino acids, about 95 amino acids to about 240 amino acids, about 95 amino acids to about 230 amino acids, about 95 amino acids to about 220 amino acids, about 95 amino acids to about 210 amino acids, about 95 amino acids to about 200 amino acids, about 95 amino acids to about 190 amino acids, about 95 amino acids to about 180 amino acids, about 95 amino acids to about 170 amino acids, about 95 amino acids to about 160 amino acids, about 95 amino acids to about 150 amino acids, about 95 amino acids to about 140 amino acids, about 95 amino acids to about 130 amino acids, about 95 amino acids to about 120 amino acids, about 95 amino acids to about 110 amino acids, about 95 amino acids to about 100 amino acids, about 100 amino acids to about 250 amino acids, about 100 amino acids to about 240 amino acids, about 100 amino acids to about 230 amino acids, about 100 amino acids to about 220 amino acids, about 100 amino acids to about 210 amino acids, about 100 amino acids to about 200 amino acids, about 100 amino acids to about 190 amino acids, about 100 amino acids to about 180 amino acids, about 100 amino acids to about 170 amino acids, about 100 amino acids to about 160 amino acids, about 100 amino acids to about 150 amino acids, about 100 amino acids to about 140 amino acids, about 100 amino acids to about 130 amino acids, about 100 amino acids to about 120 amino acids, about 100 amino acids to about 110 amino acids, about 120 amino acids to about 250 amino acids, about 120 amino acids to about 240 amino acids, about 120 amino acids to about 230 amino acids, about 120 amino acids to about 220 amino acids, about 120 amino acids to about 210 amino acids, about 120 amino acids to about 200 amino acids, about 120 amino acids to about 190 amino acids, about 120 amino acids to about 180 amino acids, about 120 amino acids to about 170 amino acids, about 120 amino acids to about 160 amino acids, about 120 amino acids to about 150 amino acids, about 120 amino acids to about 140 amino acids, about 120 amino acids to about 130 amino acids, about 130 amino acids to about 250 amino acids, about 130 amino acids to about 240 amino acids, about 130 amino acids to about 230 amino acids, about 130 amino acids to about 220 amino acids, about 130 amino acids to about 210 amino acids, about 130 amino acids to about 200 amino acids, about 130 amino acids to about 190 amino acids, about 130 amino acids to about 180 amino acids, about 130 amino acids to about 170 amino acids, about 130 amino acids to about 160 amino acids, about 130 amino acids to about 150 amino acids, about 130 amino acids to about 140 amino acids, about 140 amino acids to about 250 amino acids, about 140 amino acids to about 240 amino acids, about 140 amino acids to about 230 amino acids, about 140 amino acids to about 220 amino acids, about 140 amino acids to about 210 amino acids, about 140 amino acids to about 200 amino acids, about 140 amino acids to about 190 amino acids, about 140 amino acids to about 180 amino acids, about 140 amino acids to about 170 amino acids, about 140 amino acids to about 160 amino acids, about 140 amino acids to about 150 amino acids, about 150 amino acids to about 250 amino acids, about 150 amino acids to about 240 amino acids, about 150 amino acids to about 230 amino acids, about 150 amino acids to about 220 amino acids, about 150 amino acids to about 210 amino acids, about 150 amino acids to about 200 amino acids, about 150 amino acids to about 190 amino acids, about 150 amino acids to about 180 amino acids, about 150 amino acids to about 170 amino acids, about 150 amino acids to about 160 amino acids, about 160 amino acids to about 250 amino acids, about 160 amino acids to about 240 amino acids, about 160 amino acids to about 230 amino acids, about 160 amino acids to about 220 amino acids, about 160 amino acids to about 210 amino acids, about 160 amino acids to about 200 amino acids, about 160 amino acids to about 190 amino acids, about 160 amino acids to about 180 amino acids, about 160 amino acids to about 170 amino acids, about 170 amino acids to about 250 amino acids, about 170 amino acids to about 240 amino acids, about 170 amino acids to about 230 amino acids, about 170 amino acids to about 220 amino acids, about 170 amino acids to about 210 amino acids, about 170 amino acids to about 200 amino acids, about 170 amino acids to about 190 amino acids, about 170 amino acids to about 180 amino acids, about 180 amino acids to about 250 amino acids, about 180 amino acids to about 240 amino acids, about 180 amino acids to about 230 amino acids, about 180 amino acids to about 220 amino acids, about 180 amino acids to about 210 amino acids, about 180 amino acids to about 200 amino acids, about 180 amino acids to about 190 amino acids, about 190 amino acids to about 250 amino acids, about 190 amino acids to about 240 amino acids, about 190 amino acids to about 230 amino acids, about 190 amino acids to about 220 amino acids, about 190 amino acids to about 210 amino acids, about 190 amino acids to about 200 amino acids, about 200 amino acids to about 250 amino acids, about 200 amino acids to about 240 amino acids, about 200 amino acids to about 230 amino acids, about 200 amino acids to 220 amino acids, about 200 amino acids to about 210 amino acids, about 210 amino acids to about 250 amino acids, about 210 amino acids to about 240 amino acids, about 210 amino acids to about 230 amino acids, about 210 amino acids to about 220 amino acids, about 220 amino acids to about 250 amino acids, about 220 amino acids to about 240 amino acids, about 220 amino acids to about 230 amino acids, about 230 amino acids to about 250 amino acids, about 230 amino acids to about 240 amino acids, or about 240 amino acids to about 250 amino acids.

In some embodiments, a linker sequence between the antigen-binding domain and the transmembrane domain can be or can include one or more of an IgG1, IgG2, IgG3, or IgG4 CH1, CH2, and CH3 domain. In some embodiments, the linker between the antigen-binding domain and the transmembrane domain can be or can include CH2-CH3 human IgG1 domains. In some embodiments, the CH2-CH3 human IgG1 domains have a sequence of:

(SEQ ID NO: 62)
AEPKSPDKTHTCPPCPAPPVAGPSVFLFPPKPKDTLMIARTPEVTCWVDVS
HEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQG
NVFSCSVMHEALHNHYTQKSLSLSPGKKD.

In some embodiments, the linker sequence between the antigen-binding domain and the transmembrane domain can be or include a portion of the human CD8 extracellular sequence that is proximal to the human CD8 transmembrane domain. For example, the linker sequence between the antigen-binding domain and the transmembrane domain can be or include human CD8 sequence of TTTPAPRPPTPAPTIASQPLSLRPEACRPAAGGAVHTRGLDFACDI (SEQ ID NO: 63).

In some embodiments, the linker sequence between the antigen-binding domain and the transmembrane domain can be or include a human IgG1 hinge sequence. In some embodiments, the human IgG1 hinge sequence is AEPKSPDKTHTCPPCPKDPK (SEQ ID NO: 64).

In some embodiments, a linker sequence (e.g., any of the linker sequences described herein or known in the art) can be present between the transmembrane domain and a costimulatory domain. In some embodiments, a linker sequence (e.g., any of the linker sequences described herein or known in the art) can be present between the costimulatory domain and the ITAM.

### Nucleic Acids

Also provided herein are nucleic acids that encode any of chimeric transmembrane proteins described herein.

### Vectors

Provided herein are vectors that include any of the nucleic acids that encode any of the chimeric transmembrane proteins provided herein. A “vector” according to the present disclosure is a polynucleotide capable of inducing the expression of a recombinant protein (e.g., a chimeric transmembrane protein and/or a chimeric antigen receptor) in a mammalian cell. A vector provided herein can be, e.g., in circular or linearized form. Non-limiting examples of vectors include plasmids, SV40 vectors, adenoviral viral vectors, and adeno-associated virus (AAV) vectors. Non-limiting examples of vectors include lentiviral vectors or retroviral vectors, e.g., gamma-retroviral vectors. See, e.g., Carlens et al., Exp. Hematol. 28(10:1137-1146, 2000; Park et al., Trends Biotechnol. 29(11):550-557, 2011; and Alonso-Camino et al., Mol. Ther. Nucleic Acids 2:e93, 2013. Non-limiting examples of retroviral vectors include those derived from Moloney murine leukemia virus, myeloproliferative sarcoma virus, murine embryonic stem cell virus, murine stem cell virus, spleen focus forming virus, or adeno-associated virus. Non-limiting examples of retroviral vectors are described in, e.g., U.S. Pat. Nos. 5,219,740 and 6,207,453; Miller et al., BioTechniques 7:980-990, 1989; Miller, Human Gene Therapy 1:5-14, 1990; Scarpa et al., Virology 180:849-852, 1991; Burns et al., Proc. Natl. Acad. Sci. U.S.A. 90:8033-8037, 1993; and Boris-Lawrie et al., Cur. Opin. Genet. Develop. 3:102-109, 1993. Exemplary lentiviral vectors are described in, e.g., Wang et al., J. Immunother. 35(9):689-701, 2003; Cooper et al., Blood 101:1637-1644, 2003; Verhoeyen et al., Methods Mol. Biol. 506:97-114, 2009; and Cavalieri et al., Blood 102(2):497-505, 2003.

Exemplary vectors, in which any of the nucleic acids provided herein can be inserted, are described in, e.g., Ausubel et al., Eds. “Current Protocols in Molecular Biology” Current Protocols, 1993; and Sambrook et al., Eds. “Molecular Cloning: A Laboratory Manual,” 2nd ed., Cold Spring Harbor Press, 1989.

In some embodiments, the vectors further include a promoter sequence and/or enhancer sequence operably linked to any of the nucleic acids described herein. Non-limiting examples of promoters include promoters from human cytomegalovirus (CMV), mouse phosphoglycerate kinase 1, polyoma adenovirus, thyroid stimulating hormone a, vimentin, simian virus 40(SV40), tumor necrosis factor, β-globin, α-fetoprotein, γ-globin, β-interferon, γ-glutamyl transferase, human ubiquitin C (UBC), mouse mammary tumor virus (MMTV), Rous sarcoma virus, glyceraldehyde-3-phosphate dehydrogenase, β-actin, metallothionein II (MT II), amylase, human EF1α, cathepsin, MI muscarinic receptor, retroviral LTR (e.g. human T-cell leukemia virus HTLV), AAV ITR, interleukin-2, collagenase, platelet-derived growth factor, adenovirus E2, stromelysin, murine MX, rat insulin, glucose regulated protein 78, human immunodeficiency virus, glucose regulated protein 94, α-2-macroglobulin, MHC class I, HSP70, proliferin, immunoglobulin light chain, T-cell receptor, HLA DQα, HLA DQβ, interleukin-2 receptor, MHC class II, prealbumin (transthyretin), elastase I, albumin, c-fos, neural cell adhesion molecule (NCAM), H2B histone, rat growth hormone, human serum amyloid (SAA), muscle creatinine kinase, troponin I (TN I), and Gibbon Ape Leukemia Virus (GALV). In some embodiments, the promoter may be an inducible promoter or a constitutive promoter. Additional examples of promoters are known in the art.

In some examples, the vectors provided herein further include a poly(A) sequence, which is operably linked and positioned 3′ to the sequence encoding the chimeric transmembrane protein or a chimeric antigen receptor. Non-limiting examples of a poly(A) sequence include those derived from bovine growth hormone (Woychik et al., Proc. Natl. Acad. Sci. U.S.A. 81(13): 3944-3948, 1984, and U.S. Pat. No. 5,122,458), mouse-β-globin, mouse-α-globin (Orkin et al., EMBO J. 4(2): 453-456, 1985), human collagen, polyoma virus (Batt et al., Mol. Cell Biol. 15(9):4783-4790, 1995), the Herpes simplex virus thymidine kinase gene (HSV TK), IgG heavy chain gene polyadenylation signal (U.S. Patent Application Publication No. 2006/0040354), human growth hormone (hGH) (Szymanski et al., Mol. Therapy 15(7):1340-1347, 2007), SV40 poly(A) site, e.g., SV40 late and early poly(A) site (Schek et al., Mol. Cell Biol. 12(12):5386-5393, 1992). In some embodiments, the poly(A) sequence includes a highly conserved upstream element (AATAAA). The this AATAAA sequence can, e.g., be substituted with other hexanucleotide sequences with homology to AATAAA which are capable of signaling polyadenylation, including, e.g., ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, and AATAAG. See, e.g., WO 06012414 A2).

A poly(A) sequence can, e.g., be a synthetic polyadenylation site. See, e.g., Levitt el al, Genes Dev. 3(7): 1019-1025, 1989). In some examples, a poly(A) sequence can be the polyadenylation signal of soluble neuropilin-1: AAATAAAATACGAAATG (SEQ ID NO: 88). Additional examples of poly(A) sequences are known in the art. Additional examples and aspects of vectors are also known in the art.

In some embodiments of any of the vectors described herein, the vector can further include a sequence encoding a chimeric antigen receptor. In some embodiments, the chimeric antigen receptor can bind specifically to a tumor antigen (e.g., a tumor antigen selected from the group of glypican-3, BCMA, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin). In some embodiments, the chimeric antigen receptor comprises one or more co-stimulatory signaling domains selected from the group of 4-1BB, CD27, OX40, CD40, CD28, GITR, CD2, CD5, ICAM-1, CD11a, Lck, TNFR-I, TNFR-II, FasR, CD30, ICOS, LIGHT, NKG2C, B7-H3, DAP-10, and DAP-12. In some examples of any of the vectors described herein, the vector is a lentiviral or an adenoviral vector.

Also provided herein are sets of vectors that include a first vector that includes a sequence that encodes any of the chimeric transmembrane proteins described herein (e.g., any of the vectors that includes a sequence that encodes any of the chimeric transmembrane proteins described herein), and a second vector that includes a sequence that encodes a chimeric antigen receptor (e.g., any of the chimeric antigen receptors described herein). In some embodiments, one or both of the first vector and the second vector is a lentiviral or an adenoviral vector. In some embodiments, the second vector further includes a promoter sequence and/or an enhancer sequence that is operably linked to the sequence encoding the chimeric antigen receptor. In some embodiments, the second vector further includes a poly(A) sequence operably linked to the sequence encoding the chimeric antigen receptor.

### Methods of Introducing a Nucleic Acid or Vectors into a Mammalian Cell

A variety of different methods known in the art can be used to introduce any of the nucleic acids and vectors disclosed herein into a mammalian cell (e.g., any of the mammalian cells described herein, e.g., any of the T cells (e.g., human T cells) described herein). Non-limiting examples of methods that can be used to introduce a nucleic acid or vector into a mammalian cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection. Additional methods of introducing a nucleic acid or vector into a mammalian cell are known in the art.

### Mammalian Cells

Also provided herein are mammalian cells that include any of the nucleic acids or vectors described herein. Also provided herein are mammalian cells that include any of the sets of vectors described herein.

In some embodiments, the mammalian cell is previously obtained from a subject (e.g., a human subject, e.g., a human subject identified or diagnosed as having a cancer) or is a daughter cell of a mammalian cell that was previously obtained from a subject (e.g., a human subject, e.g., a human subject identified or diagnosed as having a cancer). In some embodiments, the mammalian cell is an immune cell. In some embodiments, the mammalian cell is a human cell.

Non-limiting examples of immune cells include a T cell (e.g., a human T cell). Non limiting examples of T cells (e.g., human T cells) include, e.g., an immature thymocyte, a peripheral blood lymphocyte, a helper T cell, a naïve T cell, a pluripotent T_{H} cell precursor, a lymphoid progenitor cell, a T_{reg} cell, a memory T cell, a T_{H}17 cell, a T_{H}22 cell, a T_{H}9 cell, a T_{H}2 cell, a T_{H}1 cell, a T_{H}3 cell, γδ T cell, an αβ T cell, a regulatory T cell (T_{reg} cell), and a tumor-infiltrating T cell. Additional examples of a T cell (e.g., a human T cell) include a CD8⁺ T cell, a CD4⁺ T cell, a memory T cell, a T_{reg} cell, natural killer cell, B cell, and a monocyte. Additional examples of mammalian cells include a mast cell, a macrophage, a neutrophil, a dendritic cell, a basophil, an cosinophil, and a natural killer cell.

### Compositions and Kits

Also provided herein are compositions (e.g., pharmaceutical compositions) that include any of the nucleic acids, vectors, sets of nucleic acids, sets of vectors, or mammalian cells described herein. For example, provided herein is a composition that includes any of the nucleic acids or sets of nucleic acids described herein, or any of the vectors or sets of vectors provided herein, and a pharmaceutically acceptable solvent or carrier.

In some embodiments, a composition can be any of the mammalian cells described herein (e.g., any of the mammalian cells described herein previously obtained from a subject, e.g., a subject identified or diagnosed as having a cancer) comprising a nucleic acid encoding any of the chimeric transmembrane proteins and/or any of the chimeric antigen receptors described herein. In a composition including any of the mammalian cells described herein, the composition can further include a cell culture medium or a pharmaceutically acceptable buffer (e.g., phosphate-buffered saline). A composition that includes any of the mammalian cells described herein can be formulated for intravenous or intraarterial administration.

Also provided are kits that include one or more of any of the compositions (e.g., pharmaceutical compositions) described herein. In some embodiments, a kit can further include instructions for performing any of the methods described herein.

### Methods of Treating a Cancer in a Subject

Also provided herein are methods of treating a cancer in a subject (e.g., a human, a mouse, a rabbit, a rat, a horse, a dog, a monkey, or an ape) that include administering a therapeutically effective amount of any of the mammalian cells including a nucleic acid encoding any of the chimeric transmembrane proteins described herein (and optionally a nucleic acid including any of the chimeric antigen receptors described herein). In some examples of these methods, the mammalian cell is a T cell (e.g., a CD8+ T cell, a CD4+ T cell, a memory T cell, a T_{reg} cell, and a natural killer T cell). In some examples, the mammalian cell (e.g., any of the mammalian cells described herein) is a mammalian cell previously obtained from a subject (e.g., a subject that has been identified or diagnosed as having a cancer, e.g., any of the cancers described herein). Some embodiments of these methods further include obtaining the mammalian cell from the subject.

Some embodiments of these methods further include introducing a nucleic acid encoding the the single-chain chimeric antigen receptor described herein or the multi-chain chimeric antigen receptor described into a mammalian cell (e.g., any of the mammalian cells described herein or known in the art) to generate the mammalian cell that is administered to the subject.

Non-limiting examples of cancer that can be treated using any of the methods provided herein include: hepatocellular carcinoma, acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

In some embodiments of any of these methods, the methods result in a decrease in the tumor burden (e.g., a decrease in tumor mass and/or volume of a solid tumor) in a subject. For example, any of the methods described herein can result in at least about 1% to about 99% (e.g., about 1% to about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 2% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 3% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 5% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, or about 10% (inclusive); about 10% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, or about 15% (inclusive); about 15% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, or about 20% (inclusive); about 20% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, or about 25% (inclusive); about 25% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, or about 30% (inclusive); about 30% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, or about 35% (inclusive); about 35% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, or about 40% (inclusive); about 40% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, or about 45% (inclusive); about 45% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, or about 50% (inclusive); about 50% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, or about 55% (inclusive); about 55% to about 99%, about 98%, about 96%, about 94%, about 92%, about 90%, about 88%, about 86%, about 84%, about 82%, about 80%, about 78%, about 76%, about 74%, about 72%, about 70%, about 65%, or about 60% (inclusive); about 60% to about 99%, about 98%, about 96%, about 94%, about 92%, about 90%, about 88%, about 86%, about 84%, about 82%, about 80%, about 78%, about 76%, about 74%, about 72%, about 70%, or about 65% (inclusive); about 65% to about 99%, about 98%, about 96%, about 94%, about 92%, about 90%, about 88%, about 86%, about 84%, about 82%, about 80%, about 78%, about 76%, about 74%, about 72%, or about 70% (inclusive); about 70% to about 99%, about 98%, about 96%, about 94%, about 92%, about 90%, about 88%, about 86%, about 84%, about 82%, about 80%, about 78%, about 76%, about 74%, or about 72% (inclusive); about 72% to about 99%, about 98%, about 96%, about 94%, about 92%, about 90%, about 88%, about 86%, about 84%, about 82%, about 80%, about 78%, about 76%, or about 74% (inclusive); about 74% to about 99%, about 98%, about 96%, about 94%, about 92%, about 90%, about 88%, about 86%, about 84%, about 82%, about 80%, about 78%, or about 76% (inclusive); about 76% to about 99%, about 98%, about 96%, about 94%, about 92%, about 90%, about 88%, about 86%, about 84%, about 82%, about 80%, or about 78% (inclusive); about 78% to about 99%, about 98%, about 96%, about 94%, about 92%, about 90%, about 88%, about 86%, about 84%, about 82%, or about 80% (inclusive); about 80% to about 99%, about 98%, about 96%, about 94%, about 92%, about 90%, about 88%, about 86%, about 84%, or about 82% (inclusive); about 82% to about 99%, about 98%, about 96%, about 94%, about 92%, about 90%, about 88%, about 86%, or about 84% (inclusive); about 84% to about 99%, about 98%, about 96%, about 94%, about 92%, about 90%, about 88%, or about 86% (inclusive); about 86% to about 99%, about 98%, about 96%, about 94%, about 92%, about 90%, or about 88% (inclusive); about 88% to about 99%, about 98%, about 96%, about 94%, about 92%, or about 90% (inclusive); about 90% to about 99%, about 98%, about 96%, about 94%, or about 92% (inclusive); about 92% to about 99%, about 98%, about 96%, or about 94% (inclusive); about 94% to about 99%, about 98%, or about 96% (inclusive); or about 96% to about 99% or about 98% (inclusive)) reduction in the tumor burden in a subject (e.g., as compared to the tumor burden in the subject prior to treatment).

In some embodiments, the methods result in a decrease in the rate of progression of a cancer in the subject. For example, any of the methods described herein can result in at least about 1% to about 99% (e.g., about 1% to about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 2% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 3% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 5% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, or about 10% (inclusive); about 10% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, or about 15% (inclusive); about 15% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, or about 20% (inclusive); about 20% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, or about 25% (inclusive); about 25% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, or about 30% (inclusive); about 30% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, or about 35% (inclusive); about 35% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, or about 40% (inclusive); about 40% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, or about 45% (inclusive); about 45% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, or about 50% (inclusive); about 50% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, or about 55% (inclusive); about 55% to about 99%, about 98%, about 96%, about 94%, about 92%, about 90%, about 88%, about 86%, about 84%, about 82%, about 80%, about 78%, about 76%, about 74%, about 72%, about 70%, about 65%, or about 60% (inclusive); about 60% to about 99%, about 98%, about 96%, about 94%, about 92%, about 90%, about 88%, about 86%, about 84%, about 82%, about 80%, about 78%, about 76%, about 74%, about 72%, about 70%, or about 65% (inclusive); about 65% to about 99%, about 98%, about 96%, about 94%, about 92%, about 90%, about 88%, about 86%, about 84%, about 82%, about 80%, about 78%, about 76%, about 74%, about 72%, or about 70% (inclusive); about 70% to about 99%, about 98%, about 96%, about 94%, about 92%, about 90%, about 88%, about 86%, about 84%, about 82%, about 80%, about 78%, about 76%, about 74%, or about 72% (inclusive); about 72% to about 99%, about 98%, about 96%, about 94%, about 92%, about 90%, about 88%, about 86%, about 84%, about 82%, about 80%, about 78%, about 76%, or about 74% (inclusive); about 74% to about 99%, about 98%, about 96%, about 94%, about 92%, about 90%, about 88%, about 86%, about 84%, about 82%, about 80%, about 78%, or about 76% (inclusive); about 76% to about 99%, about 98%, about 96%, about 94%, about 92%, about 90%, about 88%, about 86%, about 84%, about 82%, about 80%, or about 78% (inclusive); about 78% to about 99%, about 98%, about 96%, about 94%, about 92%, about 90%, about 88%, about 86%, about 84%, about 82%, or about 80% (inclusive); about 80% to about 99%, about 98%, about 96%, about 94%, about 92%, about 90%, about 88%, about 86%, about 84%, or about 82% (inclusive); about 82% to about 99%, about 98%, about 96%, about 94%, about 92%, about 90%, about 88%, about 86%, or about 84% (inclusive); about 84% to about 99%, about 98%, about 96%, about 94%, about 92%, about 90%, about 88%, or about 86% (inclusive); about 86% to about 99%, about 98%, about 96%, about 94%, about 92%, about 90%, or about 88% (inclusive); about 88% to about 99%, about 98%, about 96%, about 94%, about 92%, or about 90% (inclusive); about 90% to about 99%, about 98%, about 96%, about 94%, or about 92% (inclusive); about 92% to about 99%, about 98%, about 96%, or about 94% (inclusive); about 94% to about 99%, about 98%, or about 96% (inclusive); or about 96% to about 99% or about 98% (inclusive)) reduction in the rate of progression of a cancer in a subject (e.g., as compared to the rate of progression of a cancer in the subject prior to treatment or in a control subject or a control population of subjects having the same cancer and administered no treatment or a different treatment).

In some embodiments of any of these methods, the methods result in an increase in the time of survival of a cancer in a subject. For example, any of the methods described herein can result in an about 1% to about 800% (e.g., about 1% to about 750%, about 700%, about 650%, about 600%, about 550%, about 500%, about 450%, about 400%, about 350%, about 300%, about 250%, about 200%, about 150%, about 100%, about 80%, about 60%, about 40%, about 20%, about 10%, or about 5% (inclusive); about 5% to about 800%, about 750%, about 700%, about 650%, about 600%, about 550%, about 500%, about 450%, about 400%, about 350%, about 300%, about 250%, about 200%, about 150%, about 100%, about 80%, about 60%, about 40%, about 20%, or about 10% (inclusive); about 10% to about 800%, about 750%, about 700%, about 650%, about 600%, about 550%, about 500%, about 450%, about 400%, about 350%, about 300%, about 250%, about 200%, about 150%, about 100%, about 80%, about 60%, about 40%, or about 20% (inclusive); about 20% to about 800%, about 750%, about 700%, about 650%, about 600%, about 550%, about 500%, about 450%, about 400%, about 350%, about 300%, about 250%, about 200%, about 150%, about 100%, about 80%, about 60%, or about 40% (inclusive); about 40% to about 800%, about 750%, about 700%, about 650%, about 600%, about 550%, about 500%, about 450%, about 400%, about 350%, about 300%, about 250%, about 200%, about 150%, about 100%, about 80%, or about 60% (inclusive); about 60% to about 800%, about 750%, about 700%, about 650%, about 600%, about 550%, about 500%, about 450%, about 400%, about 350%, about 300%, about 250%, about 200%, about 150%, about 100%, about 80% (inclusive); about 80% to about 800%, about 750%, about 700%, about 650%, about 600%, about 550%, about 500%, about 450%, about 400%, about 350%, about 300%, about 250%, about 200%, about 150%, or about 100% (inclusive); about 100% to about 800%, about 750%, about 700%, about 650%, about 600%, about 550%, about 500%, about 450%, about 400%, about 350%, about 300%, about 250%, about 200%, or about 150% (inclusive); about 150% to about 800%, about 750%, about 700%, about 650%, about 600%, about 550%, about 500%, about 450%, about 400%, about 350%, about 300%, about 250%, or about 200% (inclusive); about 200% to about 800%, about 750%, about 700%, about 650%, about 600%, about 550%, about 500%, about 450%, about 400%, about 350%, about 300%, or about 250% (inclusive); about 250% to about 800%, about 750%, about 700%, about 650%, about 600%, about 550%, about 500%, about 450%, about 400%, about 350%, or about 300% (inclusive); about 300% to about 800%, about 750%, about 700%, about 650%, about 600%, about 550%, about 500%, about 450%, about 400%, or about 350% (inclusive); about 350% to about 800%, about 750%, about 700%, about 650%, about 600%, about 550%, about 500%, about 450%, or about 400% (inclusive); about 400% to about 800%, about 750%, about 700%, about 650%, about 600%, about 550%, about 500%, or about 450% (inclusive); about 450% to about 800%, about 750%, about 700%, about 650%, about 600%, about 550%, or about 500% (inclusive); about 500% to about 800%, about 750%, about 700%, about 650%, about 600%, or about 550% (inclusive); about 550% to about 800%, about 750%, about 700%, about 650%, or about 600% (inclusive); about 600% to about 800%, about 750%, about 700%, or about 650% (inclusive); about 650% to about 800%, about 750%, or about 700% (inclusive); about 700% to about 800% or about 750% (inclusive); or about 750% to about 800% (inclusive)) increase in the time of survival of a cancer in a subject (e.g., as compared to the time of survival for a control subject or a population of control subjects having the same cancer and receiving no treatment or a different treatment).

Also provided herein are methods of inducing cell death in a cancer cell in a subject in need thereof that include administering to the subject a therapeutically effective amount of any of the mammalian cells described herein.

Also provided herein are methods of decreasing the risk of developing a metastasis or an additional metastasis in a subject having a cancer that include administering to the subject a therapeutically effective amount of any of the mammalian cells described herein. For example, any of the methods described herein can result in at least about 1% to about 99% (e.g., about 1% to about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 2% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 3% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 5% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, or about 10% (inclusive); about 10% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, or about 15% (inclusive); about 15% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, or about 20% (inclusive); about 20% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, or about 25% (inclusive); about 25% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, or about 30% (inclusive); about 30% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, or about 35% (inclusive); about 35% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, or about 40% (inclusive); about 40% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, or about 45% (inclusive); about 45% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, or about 50% (inclusive); about 50% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, or about 55% (inclusive); about 55% to about 99%, about 98%, about 96%, about 94%, about 92%, about 90%, about 88%, about 86%, about 84%, about 82%, about 80%, about 78%, about 76%, about 74%, about 72%, about 70%, about 65%, or about 60% (inclusive); about 60% to about 99%, about 98%, about 96%, about 94%, about 92%, about 90%, about 88%, about 86%, about 84%, about 82%, about 80%, about 78%, about 76%, about 74%, about 72%, about 70%, or about 65% (inclusive); about 65% to about 99%, about 98%, about 96%, about 94%, about 92%, about 90%, about 88%, about 86%, about 84%, about 82%, about 80%, about 78%, about 76%, about 74%, about 72%, or about 70% (inclusive); about 70% to about 99%, about 98%, about 96%, about 94%, about 92%, about 90%, about 88%, about 86%, about 84%, about 82%, about 80%, about 78%, about 76%, about 74%, or about 72% (inclusive); about 72% to about 99%, about 98%, about 96%, about 94%, about 92%, about 90%, about 88%, about 86%, about 84%, about 82%, about 80%, about 78%, about 76%, or about 74% (inclusive); about 74% to about 99%, about 98%, about 96%, about 94%, about 92%, about 90%, about 88%, about 86%, about 84%, about 82%, about 80%, about 78%, or about 76% (inclusive); about 76% to about 99%, about 98%, about 96%, about 94%, about 92%, about 90%, about 88%, about 86%, about 84%, about 82%, about 80%, or about 78% (inclusive); about 78% to about 99%, about 98%, about 96%, about 94%, about 92%, about 90%, about 88%, about 86%, about 84%, about 82%, or about 80% (inclusive); about 80% to about 99%, about 98%, about 96%, about 94%, about 92%, about 90%, about 88%, about 86%, about 84%, or about 82% (inclusive); about 82% to about 99%, about 98%, about 96%, about 94%, about 92%, about 90%, about 88%, about 86%, or about 84% (inclusive); about 84% to about 99%, about 98%, about 96%, about 94%, about 92%, about 90%, about 88%, or about 86% (inclusive); about 86% to about 99%, about 98%, about 96%, about 94%, about 92%, about 90%, or about 88% (inclusive); about 88% to about 99%, about 98%, about 96%, about 94%, about 92%, or about 90% (inclusive); about 90% to about 99%, about 98%, about 96%, about 94%, or about 92% (inclusive); about 92% to about 99%, about 98%, about 96%, or about 94% (inclusive); about 94% to about 99%, about 98%, or about 96% (inclusive); or about 96% to about 99% or about 98% (inclusive)) decrease in the risk of developing a metastasis or an additional metastasis in the subject (e.g., as compared to the risk of developing a metastasis or an additional metastasis in a control subject or a control population of subjects having the same cancer and administered no treatment or a different treatment).

### EXAMPLES

The invention is further described in the following examples, which do not limit the scope of the invention described in the claims.

### Example 1. Contruction of a Vector Containing a Chimeric Transmembrane Protein and a CAR

The vector shown in FIG. 2 was generated. The amino acid and nucleic acid sequences present in the vector in FIG. 2 are provided below.

mbIL2 (Chimeric Transmembrane Protein of SEQ ID NO:
7) (CSF2RA signal peptide underlined, poly-His tag
in bold)
MLLLVTSLLLCELPHPAFLLIPHHHHHHAPTSSSTKKTQLQLEHLLLDLQM
ILNGINNYKNPKLTRMLTFKFYMPKKATELKHLQCLEEELKPLEEVLNLAQ
SKNFHLRPRDLISNINVIVLELKGSETTFMCEYADETATIVEFLNRWITFC
QSllSTLTSGGGSGGGGSGGGGSGGGGSGGGSLQELCDDDPPEIPHATFKA
MAYKEGTMLNCECKRGFRRIKSGSLYMLCTGNSSHSSWDNQCQCTSSATRN
TTKQVTPQPEEQKERKTTEMQSPMQPVDQASLPGHCREPPPWENEATERIY
HFVVGQMVYYQCVQGYRALHRGPAESVCKMTHGKTRWTQPQLICTGEMETS
QFPGEEKPQASPEGRPESETSCLVTTTDFQIQTEMAATMETSIFTTEYQVA
VAGCVFLLISVLLLSGLTWQRRQRKSRRTI
Nucleic Acid Sequence Encoding mbIL2
(SEQ ID NO: 65)
ATGCTCCTGCTCGTGACTTCACTTCTTCTCTGTGAACTCCCACACCCCGCG
TTTTTGCTTATCCCTcatcatcaccatcaccacGCACCTACTTCAAGTTCT
ACAAAGAAAACACAGCTACAACTGGAGCATTTACTTCTGGATTTACAGATG
ATTTTGAATGGAATTAATAATTACAAGAATCCCAAACTCACCAGGATGCTC
ACATTTAAGTTTTACATGCCCAAGAAGGCCACAGAACTGAAACATCTTCAG
TGTCTAGAAGAAGAACTCAAACCTCTGGAGGAAGTGCTAAATTTAGCTCAA
AGCAAAAACTTTCACTTAAGACCCAGGGACTTAATCAGCAATATCAACGTA
ATAGTTCTGGAACTAAAGGGATCTGAAACAACATTCATGTGTGAATATGCT
GATGAGACAGCAACCATTGTAGAATTTCTGAACAGATGGATTACCTTTTGT
CAAAGCATCATCTCAACACTGACTTCTGGAGGAGGTAGTGGAGGCGGCGGG
AGCGGTGGGGGAGGGTCTGGGGGTGGGGGATCCGGAGGTGGGTCACTGCAG
GAGCTCTGTGACGATGACCCGCCAGAGATCCCACACGCCACATTCAAAGCC
ATGGCCTACAAGGAAGGAACCATGTTGAACTGTGAATGCAAGAGAGGTTTC
CGCAGAATAAAAAGCGGGTCACTCTATATGCTCTGTACAGGAAACTCTAGC
CACTCGTCCTGGGACAACCAATGTCAATGCACAAGCTCTGCCACTCGGAAC
ACAACGAAACAAGTGACACCTCAACCTGAAGAACAGAAAGAAAGGAAAACC
ACAGAAATGCAAAGTCCAATGCAGCCAGTGGACCAAGCGAGCCTTCCAGGT
CACTGCAGGGAACCTCCACCATGGGAAAATGAAGCCACAGAGAGAATTTAT
CATTTCGTGGTGGGGCAGATGGTTTATTATCAGTGCGTCCAGGGATACAGG
GCTCTACACAGAGGTCCTGCTGAGAGCGTCTGCAAAATGACCCACGGGAAG
ACAAGGTGGACCCAGCCCCAGCTCATATGCACAGGTGAAATGGAGACCAGT
CAGTTTCCAGGTGAAGAGAAGCCTCAGGCAAGCCCCGAAGGCCGTCCTGAG
AGTGAGACTTCCTGCCTCGTCACAACAACAGATTTTCAAATACAGACAGAA
ATGGCTGCAACCATGGAGACGTCCATATTTACAACAGAGTACCAGGTAGCA
GTGGCCGGCTGTGTTTTCCTGCTGATCAGCGTCCTCCTCCTGAGTGGGCTC
ACCTGGCAGCGGAGACAGAGGAAGAGTAGAAGAACAATCTG
AA Sequence for T2A and Furin Cleavage site
(SEQ ID NO: 66)
RKRRGKPIPNPLLGLDSTSGSGEGRGSLLTCGDVEENPGPG
Nucleic Acid Sequence for T2A and Furin Cleavage
site
(SEQ ID NO: 67)
cggaagagaagaggcaagcccatccccaacccactgctgggcctggatagc
acctccggctcaggagagggcagaggctctctgctgacctgcggcgacgtg
gaagagaacccgggcccgggc
FMC63-41BB-CD3z CAR Protein Sequence
(SEQ ID NO: 68)
MLLLVTSLLLCELPHPAFLLIPEQKLISEEDLDIQMTQTTSSLSASLGDRV
TISCRASQDISKYLNWYQQKPDGTVKLLIYHTSRLHSGVPSRFSGSGSGTD
YSLTISNLEQEDIATYFCQQGNTLPYTFGGGTKLEITGGGGSGGGGSGGGG
SEVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYYY
GGSYAMDYWGQGTSVTVSSGSTTTPAPRPPTPAPTIASQPLSLRPEACRPA
AGGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYS
EIGMKGERRRGKGHDGLYQGLSTATKDTYDALHMQALPPR
FMC63-41BB-CD3z CAR Nucleic Acid Sequence
(SEQ ID NO: 69)
ATGCTTCTCCTGGTGACAAGCCTTCTGCTCTGTGAGTTACCACACCCAGCA
TTCCTCCTGATTCCTGAACAGAAGCTGATAAGTGAGGAGGACTTGGACATC
CAGATGACCCAGACCACCAGCAGCCTGAGCGCCAGCCTGGGCGATAGAGTG
ACCATCAGCTGCAGAGCCAGCCAGGACATCAGCAAGTACCTGAACTGGTAT
CAGCAGAAACCCGACGGCACCGTGAAGCTGCTGATCTACCACACCAGCAGA
CTGCACAGCGGCGTGCCCAGCAGATTTTCTGGCAGCGGCTCCGGCACCGAC
TACAGCCTGACCATCTCCAACCTGGAACAGGAAGATATCGCTACCTACTTC
TGTCAGCAAGGCAACACCCTGCCCTACACCTTCGGCGGAGGCACCAAGCTG
GAAATCACAGGCGGCGGAGGATCTGGCGGAGGCGGAAGTGGCGGAGGGGGA
TCTGAAGTGAAACTGCAGGAAAGCGGCCCTGGCCTGGTGGCCCCATCTCAG
TCTCTGAGCGTGACCTGTACCGTGTCCGGCGTGTCCCTGCCTGACTATGGC
GTGTCCTGGATCAGACAGCCCCCCAGAAAGGGCCTGGAATGGCTGGGAGTG
ATCTGGGGCAGCGAGACAACCTACTACAACAGCGCCCTGAAGTCCCGGCTG
ACCATCATCAAGGACAACTCCAAGAGCCAGGTGTTCCTGAAGATGAACAGC
CTGCAGACCGACGACACCGCCATCTACTACTGCGCCAAGCACTACTACTAC
GGCGGCAGCTACGCCATGGACTACTGGGGCCAGGGCACAAGCGTGACCGTG
TCTAGCGGATCCACCACGACGCCAGCGCCGCGACCACCAACACCGGCGCCC
ACCATCGCGTCGCAGCCCCTGTCCCTGCGCCCAGAGGCGTGCCGGCCAGCG
GCGGGGGGCGCAGTGCACACGAGGGGGCTGGACTTCGCCTGTGATATCTAC
ATCTGGGCGCCCTTGGCCGGGACTTGTGGGGTCCTTCTCCTGTCACTGGTT
ATCACCCTTTACTGCAAACGGGGCAGAAAGAAACTCCTGTATATATTCAAA
CAACCATTTATGAGACCAGTACAAACTACTCAAGAGGAAGATGGCTGTAGC
TGCCGATTTCCAGAAGAAGAAGAAGGAGGATGTGAACTGAGAGTGAAGTTC
AGCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAGA
CGTGGCCGGGACCCTGAGATGGGGGGAAAGCCGAGAAGGAAGAACCCTCAG
GAAGGCCTGTACAATGAACTGCAGAAAGATAAGATGGCGGAGGCCTACAGT
GAGATTGGGATGAAAGGCGAGCGCCGGAGGGGCAAGGGGCACGATGGCCTT
TACCAGGGTCTCAGTACAGCCACCAAGGACACCTACGACGCCCTTCACATG
CAGGCCCTGCCCCCTCGC
CAR Signal Peptide- Amino Acid Sequence
(SEQ ID NO: 70)
MLLLVTSLLLCELPHPAFLLIP
CAR Signal Peptide- Nucleic Acid Sequence
(SEQ ID NO: 71)
ATGCTTCTCCTGGTGACAAGCCTTCTGCTCTGTGAGTTACCACACCCAGCA
TTCCTCCTGATTCCT
CAR Myc Tag- Amino Acid Sequences
(SEQ ID NO: 72)
EQKLISEEDL
CAR Myc Tag- Nucleic Acid Sequences
(SEQ ID NO: 73)
GAACAGAAGCTGATAAGTGAGGAGGACTTG
FMC63 scFv- Amino Acid Sequence
(SEQ ID NO: 74)
DIQMTQTTSSLSASLGDRVTISCRASQDISKYLNWYQQKPDGTVKLLIYHT
SRLHSGVPSRFSGSGSGTDYSLTISNLEQEDIATYFCQQGNTLPYTFGGGT
KLEITGGGGSGGGGSGGGGSEVKLQESGPGLVAPSQSLSVTCTVSGVSLPD
YGVSWIRQPPRKGLEWLGVIWGSETTYYNSALKSRLTIIKDNSKSQVFLKM
NSLQTDDTAIYYCAKHYYYGGSYAMDYWGQGTSVTVSS
FMC63 scFv- Nucleic Acid Sequence
(SEQ ID NO: 75)
GACATCCAGATGACCCAGACCACCAGCAGCCTGAGCGCCAGCCTGGGCGAT
AGAGTGACCATCAGCTGCAGAGCCAGCCAGGACATCAGCAAGTACCTGAAC
TGGTATCAGCAGAAACCCGACGGCACCGTGAAGCTGCTGATCTACCACACC
AGCAGACTGCACAGCGGCGTGCCCAGCAGATTTTCTGGCAGCGGCTCCGGC
ACCGACTACAGCCTGACCATCTCCAACCTGGAACAGGAAGATATCGCTACC
TACTTCTGTCAGCAAGGCAACACCCTGCCCTACACCTTCGGCGGAGGCACC
AAGCTGGAAATCACAGGCGGCGGAGGATCTGGCGGAGGCGGAAGTGGCGGA
GGGGGATCTGAAGTGAAACTGCAGGAAAGCGGCCCTGGCCTGGTGGCCCCA
TCTCAGTCTCTGAGCGTGACCTGTACCGTGTCCGGCGTGTCCCTGCCTGAC
TATGGCGTGTCCTGGATCAGACAGCCCCCCAGAAAGGGCCTGGAATGGCTG
GGAGTGATCTGGGGCAGCGAGACAACCTACTACAACAGCGCCCTGAAGTCC
CGGCTGACCATCATCAAGGACAACTCCAAGAGCCAGGTGTTCCTGAAGATG
AACAGCCTGCAGACCGACGACACCGCCATCTACTACTGCGCCAAGCACTAC
TACTACGGCGGCAGCTACGCCATGGACTACTGGGGCCAGGGCACAAGCGTG
ACCGTGTCTAGC
CAR CD8 Hinge and Transmembrane Domain- Amino Acid
Sequence
(SEQ ID NO: 76)
GSTTTPAPRPPTPAPTIASQPLSLRPEACRPAAGGAVHTRGLDFACDIYIW
APLAGTCGVLLLSLVITLYC
CAR CD8 Hinge and Transmembrane Domain- Nucleic
Acid Sequence
(SEQ ID NO: 77)
GGATCCACCACGACGCCAGCGCCGCGACCACCAACACCGGCGCCCACCATC
GCGTCGCAGCCCCTGTCCCTGCGCCCAGAGGCGTGCCGGCCAGCGGCGGGG
GGCGCAGTGCACACGAGGGGGCTGGACTTCGCCTGTGATATCTACATCTGG
GCGCCCTTGGCCGGGACTTGTGGGGTCCTTCTCCTGTCACTGGTTATCACC
CTTTACTGC
CAR 4-1BB Costimulatory Domain- Amino Acid Sequence
(SEQ ID NO: 78)
KRGRKKLLYIFKQPFMRPVQTTQEEDGCSCRFPEEEEGGCE
CAR 4-1BB Costimulatory Domain- Nucleic Acid
Sequence
(SEQ ID NO: 79)
AAACGGGGCAGAAAGAAACTCCTGTATATATTCAAACAACCATTTATGAGA
CCAGTACAAACTACTCAAGAGGAAGATGGCTGTAGCTGCCGATTTCCAGAA
GAAGAAGAAGGAGGATGTGAA
CAR CD3z Domain- Amino Acid Sequence
(SEQ ID NO: 80)
LRVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDTY
DALHMQALPPR
CAR CD3z Domain- Nucleic Acid Sequence
(SEQ ID NO: 81)
CTGAGAGTGAAGTTCAGCAGGAGCGCAGACGCCCCCGCGTACCAGCAGGGC
CAGAACCAGCTCTATAACGAGCTCAATCTAGGACGAAGAGAGGAGTACGAT
GTTTTGGACAAGAGACGTGGCCGGGACCCTGAGATGGGGGGAAAGCCGAGA
AGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTAC
GACGCCCTTCACATGCAGGCCCTGCCCCCTCGC

### Example 2. Transduced T-Cells Expressing Both a Chimeric Transmembrane Protein and CAR

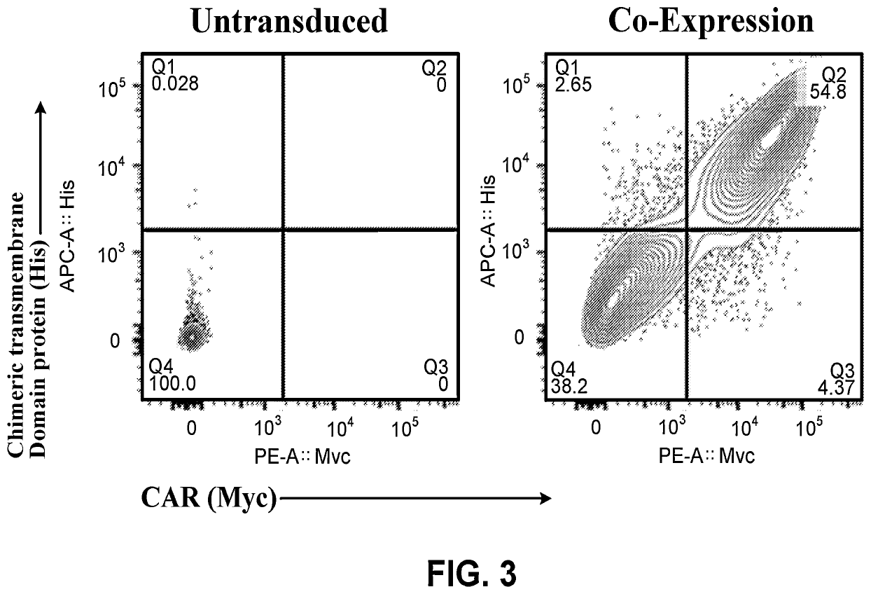

To determine correct cell surface expression of both chimeric proteins (mbIL2 and FMC63-41BB-CD3z CAR) by flow cytometry, T-cells were stained with fluorochrome conjugated antibodies against the His and Myc tags present at the N-terminus of the mbIL2 chimeric transmembrane protein and FMC63-41BB-CD3z CAR, respectively. Briefly, up to 1×10⁶ primary T-cells were harvested 72 hours post-transduction with the lentiviral vector shown in FIG. 2 and incubated with anti-His (APC; 1:100) and anti-Myc (PE; 1:100) antibodies in PBS+2% FBS (FACS Buffer) for 30 minutes at 4° C. Flow cytometry was performed using BD LSRII Fortessa instruments and analyzed using FlowJo V10 software. The double positive (Myc-PE+, His-APC) population demonstrates co-expression of both chimeric proteins (mbIL2 and FMC63-41BB-CD3z CAR) in primary human T-cells (FIG. 3).

### Example 3. Co-Expression of a Chimeric Transmembrane Protein and a CAR Promotes Maintenance of CD62L+ Memory T-Cell Populations

IL-2 signaling through JAK3 and STAT5 will cause changes to the phenotype of primary human T-cells, including skewing the CD4 to CD8 ratio toward CD8⁺ effector cells during ex vivo culture. In addition, IL-2 signaling is typically associated with CD8⁺ T cell differentiation toward terminal effector T cells and augments the cytotoxic response against target cells. To determine the effect of the chimeric transmembrane protein and CAR expression on the phenotype of primary human T-cells, primary human T-cells expressing none, one, or both of mbIL2 and FMC63-41BB-CD3z CAR were stained with fluorchrome-conjugated antibodies against multiple cell surface markers. Briefly, up to 1×10⁶ primary human T-cells were harvested 14 days post activation and incubated with anti-Myc (FITC; 1:100), anti-CD4 (BV785; 1:200), anti-CD8 (PE; 1:200), anti-CD45RO (APC; 1:200), and anti CD62L (BV605; 1:200) antibodies in PBS+2% FBS (FACS Buffer) for 30 minutes at 4° C. Flow cytometry was performed using BD LSRII Fortessa instruments and analyzed using FlowJo V10 software. The CD4⁺ and CD8⁺ T-cell populations shown are gated on Myc⁺ populations, while T-cell memory subset populations (CD62L vs CD45RO) are gated on Myc⁺, CD8⁺ populations.

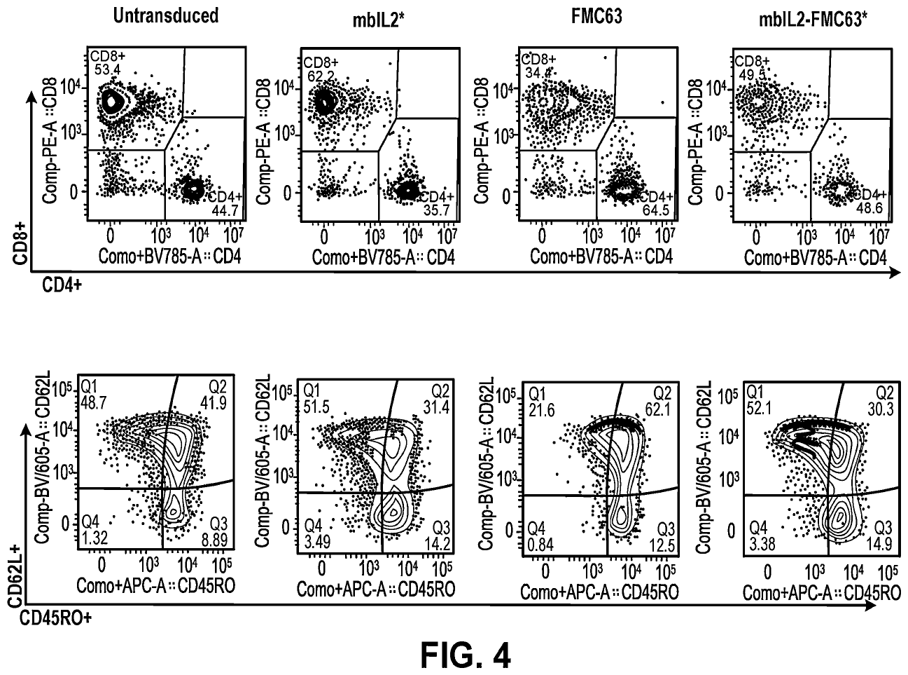

Expression of the mbIL2 and FMC63-41BB-CD3z CAR drives expansion of CD8⁺ T-cells and preserves stem cell memory populations (CD62L⁺, CD45RO⁻), while increasing effector memory populations (CD62L⁻, CD45RO⁺), relative to T-cells that express FMC63-41BB-CD3z CAR alone (FIG. 4).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A chimeric transmembrane protein comprising:
an extracellular IL-2 domain;
an extracellular sushi domain from an alpha chain of interleukin-2 receptor; and
a transmembrane domain of an alpha chain of interleukin-2 receptor.

2. The chimeric transmembrane protein of claim 1, wherein the extracellular IL-2 domain comprises a sequence that is at least 80% identical to the sequence of a wildtype IL-2 protein.

3. The chimeric transmembrane protein of claim 1 or 2, wherein the chimeric transmembrane protein further comprises a linker sequence positioned between the extracellular IL-2 domain and the extracellular sushi domain.

4. The chimeric transmembrane protein of any one of claims 1-3, further comprising an additional linker sequence positioned between the extracellular sushi domain and the transmembrane domain.

5. The chimeric transmembrane protein of any one of claims 1-4, wherein the extracellular sushi domain is at least 80% identical to a sequence of an extracellular sushi domain of a wildtype alpha chain of interleukin-2 receptor.

6. The chimeric transmembrane protein of any one of claims 1-5, wherein the transmembrane domain is at least 80% identical to a sequence of a transmembrane domain of a wildtype alpha chain of interleukin-2 receptor.

7. The chimeric transmembrane protein of any one of claims 1-6, wherein the chimeric transmembrane protein further comprises an intracellular domain of an alpha chain of interleukin-2 receptor.

8. A nucleic acid encoding a chimeric transmembrane protein of any one of claims 1-7.

9. An vector comprising the nucleic acid of claim 8.

10. The vector of claim 9, further comprising a sequence encoding a chimeric antigen receptor.

11. A mammalian cell comprising a nucleic acid of claim 8 or a vector of claim 9 or 10.

12. A pharmaceutical composition comprising:
a vector of claim 9 or 10 and a pharmaceutically acceptable carrier; or
a mammalian cell of claim 11.

13. A set of vectors comprising:
a first vector that is a vector of claim 9; and
a second vector that comprises a sequence encoding a chimeric antigen receptor.

14. A mammalian cell comprising a set of vectors of claim 13.

15. The mammalian cell of claim 11 or 14, wherein the mammalian cell is an immune cell.

16. A pharmaceutical composition comprising:
a mammalian cell of claim 14 or 15; or
a set of vectors of claim 13 and a pharmaceutically acceptable carrier.

17. A method of treating a cancer in a subject in need thereof, the method comprising:
administering a therapeutically effective amount of a mammalian cell of claim 11, 14, or 15 to the subject.

18. A method of reducing the volume of a solid tumor in a subject in need thereof, the method comprising:
administering a therapeutically effective amount of a mammalian cell of claim 11, 14, or 15 to the subject.

19. A method of inducing cell death in a cancer cell in a subject in need thereof, the method comprising:
administering a therapeutically effective amount of a mammalian cell of claim 11, 14, or 15 to the subject.

20. A method of decreasing the risk of developing a metastasis or an additional metastasis in a subject having a cancer, the method comprising:
administering a therapeutically effective amount of a mammalian cell of claim 11, 14, or 15 to the subject.


SEQUENCE LISTING (81 sequences)

SEQ ID NO: 1 - protein, 133 aa, Homo sapiens [sequence omitted]

SEQ ID NO: 2 - protein, 23 aa, synthetic construct
  REGION 1..23: note = synthetic linker sequence
GGGSGGGGSGGGGSGGGGSGGGS

SEQ ID NO: 3 - protein, 63 aa, Homo sapiens
ELCDDDPPEIPHATFKAMAYKEGTMLNCECKRGFRRIKSGSLYMLCTGNSSHSSWDNQCQCTS

SEQ ID NO: 4 - protein, 64 aa, Homo sapiens
GHCREPPPWENEATERIYHFVVGQMVYYQCVQGYRALHRGPAESVCKMTHGKTRWTQPQLICTG

SEQ ID NO: 5 - protein, 19 aa, Homo sapiens
VAVAGCVFLLISVLLLSGL

SEQ ID NO: 6 - protein, 13 aa, Homo sapiens
TWQRRQRKSRRTI

SEQ ID NO: 7 - protein, 438 aa, synthetic construct
  REGION 1..438: note = synthetic mbIL2 sequence
MLLLVTSLLLCELPHPAFLLIPHHHHHHAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGSGGGGSGGGGSGGGGSGGGSLQELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 8 - protein, 410 aa, synthetic construct
  REGION 1..410: note = synthetic chimeric transmembrane protein
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGSGGGGSGGGGSGGGGSGGGSLQELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 9 - protein, 22 aa, synthetic construct
  REGION 1..22: note = synthetic signal sequence
MLLLVTSLLLCELPHPAFLLIP

SEQ ID NO: 10 - DNA, 822 bp, Homo sapiens
  source 1..822: mol_type = genomic DNA
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 11 - protein, 134 aa, Macaca mulatta
APTSSSTKKTQLQLEHLLLDLQMILNGINNYKNPKLTRMLTFKFYMPKKATELKHLQCLEEELKPLEEVLNLAQSKNFHLRDTKDLISNINVIVLELKGSETTLMCEYADETATIVEFLNRWITFCQSIISTLT

SEQ ID NO: 12 - DNA, 497 bp, Macaca mulatta
  source 1..497: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaattgaaacatcttcagtgtctagaagaagaactcaaacctctggaggaagtgctaaatttagctcaaagcaaaaactttcacttaagagataccaaggacttaatcagcaatatcaacgtaatagttctggaactaaagggatctgaaacaacactgatgtgtgaatatgctgatgagacagcaaccattgtagaatttctgaacagatggattaccttttgtcaaagcatcatctcaacactgacctgataattaagtgcttcccacttaaaacatatcag

SEQ ID NO: 13 - protein, 121 aa, Gallus gallus
ASLSSEKWKTLQTLIKDLEILENIKNKIHLELYTPTETQECTQQTLQCYLGEVVTLKKETEDDTEIKEEFVTAIQNIEKNLKSLTGLNHTGSECKICEANNKKKFPDFLHELTNFVRYLQK

SEQ ID NO: 14 - DNA, 798 bp, Gallus gallus
  source 1..798: mol_type = genomic DNA
atcacctcacattgtacatcacaatttgaataccagcatacagataactgggacactgccatgatgtgcaaagtactgatctttggctgtatttcggtagcaatgctaatgactacagcttatggagcatctctatcatcagaaaaatggaaaactcttcaaacattaataaaggatttagaaatattggaaaatatcaagaataagattcatctcgagctctacacaccaactgagacccaggagtgcacccagcaaactctgcagtgttacctgggagaagtggttactctgaagaaagaaactgaagatgacactgaaattaaagaagaatttgtaactgctattcaaaatatcgaaaagaacctcaagagtcttacgggtctaaatcacaccggaagtgaatgcaagatctgtgaagctaacaacaagaaaaaatttcctgattttctccatgaactgaccaactttgtgagatatctgcaaaaataagcaactaatcatttttattttactgctatgttatttatttaattatttaattacagataatttatatattttatcccgtggctaactaatctgctgtccattctgggaccactgtatgctcttagtctgggtgatatgacgtctgttctaagatcatatttgatcctttctgtaagccctacgggctcaaaatgtacgttggaaaactgattgattctcactttgtcggtaaagtgatatgtgtttactgaaagaatttttaaaagtcacttctagatgacatttaataaatttcagtaatatatg

SEQ ID NO: 15 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 16 - DNA, 955 bp, Mus musculus
  source 1..955: mol_type = genomic DNA
tatcacccttgctaatcactcctcacagtgacctcaagtcctgcaggcatgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataactatgtacctcctgcttacaacacataaggctctctatttatttaaatatttaactttaatttatttttggatgtattgtttactatcttttgtaactactagtcttcagatgataaatatggatctttaaagattctttttgtaagccccaagggctcaaaaatgttttaaactatttatctgaaattatttattatattgaattgttaaatatcatgtgtaggtagactcattaataaaagtatttagatgattcaaatataaataagctcagatgtctgtcatttttaggacagcacaaagtaagcgctaaaataacttctcagttattcctgtgaactctatgttaatcagtgttttcaagaaataaagctctcctctaaaaaaaaaaaaaaa

SEQ ID NO: 17 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 18 - DNA, 3216 bp, Homo sapiens
  source 1..3216: mol_type = genomic DNA
ggcagtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagatgagcataccagcagtccttgaacattatcaaaaagttcagcatattagaatcaccgggaggccttgttaaaagagttcgctgggcccatcttcagagtctctgagttgttggtctggaatagagccaaatgttttgtgtgtctaacaattcccaggtgctgttgctgctgctactattccaggaacacactttgagaaccattgtgttattgctctgcacgcccacccactctcaactcccacgaaaaaaatcaacttccagagctaagatttcggtggaagtcctggttccatatctggtgcaagatctcccctcacgaatcagttgagtcaacattctagctcaacaacatcacacgattaacattaacgaaaattattcatttgggaaactatcagccagttttcacttctgaaggggcaggagagtgttatgagaaatcacggcagttttcagcagggtccagattcagattaaataactattttctgtcatttctgtgaccaaccacatacaaacagactcatctgtgcactctccccctcccccttcaggtatatgttttctgagtaaagttgaaaagaatctcagaccagaaaatatagatatatatttaaatcttacttgagtagaactgattacgacttttgggtgttgaggggtctataagatcaaaacttttccatgataatactaagatgttatcgaccatttatctgtccttctctcaaaagtgtatggtggaattttccagaagctatgtgatacgtgatgatgtcatcactctgctgttaacatataataaatttattgctattgtttataaaagaataaatgatatttttt

SEQ ID NO: 19 - protein, 244 aa, Pan troglodytes
ELCDDDPPEITHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQTVYYQCVQGYRALHRGPAESVCKMTHGKTRWTQPQLICTGEMETSQFPGEEKPQASPEGRPESETSCLITTTDFQIQTEMAATMETFIFTTEYQVAVAGCVFLLISVLLLSGLTWQRRQ

SEQ ID NO: 20 - DNA, 798 bp, Pan troglodytes
  source 1..798: mol_type = genomic DNA
atggattcatacctgctgatgtggggactgctcacgctcatcatggtgcctggctgctttgcagagctctgtgacgatgacccgccagagatcacacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccgtgggaaaatgaagccacagagagaatttatcatttcgtggtggggcagacggtttactaccagtgcgtccagggatacagggctctacacagaggtcctgctgagagcgtctgcaaaatgacccatgggaagacaaggtggacccagccccagctcatatgcacaggtgaaatggagaccagtcagtttccaggtgaagagaagcctcaggcaagccccgaaggccgtcctgagagtgagacttcctgcctcatcacaacaacagattttcaaatacagacagaaatggctgcaaccatggagacgttcatatttacaacagagtaccaggtagcagtggccggctgtgttttcctgctgatcagcgtcctcctcctgagtgggctcacctggcagcggagacagtaa

SEQ ID NO: 21 - protein, 251 aa, Macaca mulatta
ELCDDDPPKITHATFKAVAYKEGTMLNCECKRGFRRIKSGSPYMLCTGNSSHSSWDNQCQCTSSAARNTTKQVTPQPEEQKERKTTEMQSQMQLADQVSLPGHCREPPPWENEATERIYHFVVGQMVYYQCVQGYRALHRGPAESICKMTHGKTRWTQPQLICTGETEPSQFPGEEEPQASPDGLPESETSRLVTTTDFRIQTEVAATMETFIFTTEYQVAVAGCVFLLISVLLLSGLTWQRRQRKNRRTI

SEQ ID NO: 22 - DNA, 934 bp, Macaca mulatta
  source 1..934: mol_type = genomic DNA
atggatccatacctgctcatgtggggactgctcacgttcatcacggtgcctggctgccaggcagagctctgtgacgatgacccgccaaaaatcacacatgccacattcaaagccgtggcctacaaggaaggaaccatgttgaactgtgaatgcaagagaggtttccgcagaataaaaagcgggtcaccctatatgctctgtacaggaaactctagccactcgtcctgggacaaccaatgtcaatgcacaagctctgctgctcggaacacaacaaaacaagtgacacctcaacctgaagaacagaaagaaagaaaaaccacagaaatgcaaagtcaaatgcagctggcggaccaagtgagccttccaggtcactgcagggaacctccaccgtgggaaaatgaagccacagaaagaatttatcatttcgtggtggggcagatggtttactaccagtgcgtccagggatacagggctctacacagaggtcctgctgagagcatctgcaaaatgacccacgggaagacaagatggacccagccccagctcatatgcacaggtgaaacggagcccagtcagtttccaggtgaagaggagcctcaggcaagccccgacggccttcctgagagtgagacttcccgcctcgtcacaacaacagattttcgaatacagacagaagtggctgcaaccatggaaacgttcatatttacaacagagtaccaagtagcagtggccggctgtgttttcctgctgatcagcgtcctcctgctgagtgggctcacctggcagcggagacagaggaagaatagaagaacaatctagaaaaccaaaagaacaagaacttcttggtaagaagccgagaacagacaacagaagtcatgaagcccaagcgaaatcaaaggtgctaaatgcttgcccaggagacatccgttgtgct

SEQ ID NO: 23 - protein, 247 aa, Mus musculus
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKVAVASCLFLLISILLLSGLTWQHRWRKSRRTI

SEQ ID NO: 24 - DNA, 4428 bp, Mus musculus
  source 1..4428: mol_type = genomic DNA
aagttcctgctgagcagatcagcctaatgcttaaatagaacaactcctggctgtcattgacattgtctaaaagccaagatgacagactgagaggcctgagcccttgttctggcattctcccaggaagatgcagtaaaggggttgacccaatatactgcagagaatttcatccagttccctcctccatcctgatcccatgtgccaggaagatggagccacgcttgctgatgttggggtttctctcattaaccatagtacccagttgtcgggcagaactgtgtctgtatgacccacccgaggtccccaatgccacattcaaagccctctcctacaagaacggcaccatcctaaactgtgaatgcaagagaggtttccgaagactaaaggaattggtctatatgcgttgcttaggaaactcctggagcagcaactgccagtgcaccagcaactcccatgacaaatcgagaaagcaagttacagctcaacttgaacaccagaaagagcaacaaaccacaacagacatgcagaagccaacacagtctatgcaccaagagaaccttacaggtcactgcagggagccacctccttggaaacatgaagattccaagagaatctatcatttcgtggaaggacagagtgttcactacgagtgtattccgggatacaaggctctacagagaggtcctgctattagcatctgcaagatgaagtgtgggaaaacggggtggactcagccccagctcacatgtgtagatgaaagagaacaccaccgatttctggctagtgaggaatctcaaggaagcagaaattcttctcccgagagtgagacttcctgccccataaccaccacagacttcccacaacccacagaaacaactgcaatgacggagacatttgtgctcacaatggagtataaggtagcagtggccagctgcctcttcctgctcatcagcatcctcctcctgagcgggctcacctggcaacacagatggaggaagagcagaagaaccatctagcaagctagaaaagtcagagcccaggcaagcggatgggaatcacaaagctcaagccaaatctgagacgccaagcattcacctaacggctgtttccttctgatccctgggtttctagaacattctgaagtcacaggacataacagcaactctatcactaaactggactttgccattgaagaataggatctaaccacttcagcacagcagttctaaagctttaatgggagagagggcccaacagtgctctgtgtgttttgtttttgtgtatatctgttgatgggagctgagatggtgtggtcacttttcatgtaacatatagtatagaaaaagtagctttaggttgacttcattgttacaacccagtttggaaagcccaagtaaaactcagcactaatgtaaataattcctcctcctcctcctctctcttttcatcctccgctccatcttcctcttcttcctcctccttttccacctcctctgtccctacccacccccacccatccactttccttcttcctttctgctctcacaagctcatcctagctacacgtgcatggctggctcctttttcaacctctgtttgcctaactggctcttctgatttcatcacttactgatcagcctttaaaactctgagctggcaaagatgactctatctatgttcttggctcagtcccagaaggaaacccccttttcatgaagcttcagttttgacatcctgaagaacagaaactgtggcagaacaatcttcagataacatcaaaacaaagtggagaagccacgggaactgtggagctctggtattcagaagcctgtgtctagggtctgcgccaggagcagaaggctgaaggaagtcccaggacgtggacttagatgctttcccagcaggccactctaagcgctggtttctttgggacagctgtcaattgtacgctcaatttagcctgcactaatctgatgcttacaggtgaacactcaaggcacaggtatggacttggtacataccgtgaaaacactggaaagaaaagaatactttcaagtttacagaaggaaggaaggaaaaaggaagcagaggtggtgattatacaaaagattagctgtagactggatatcccaggcatcctcggataatgcccccgccccagcaccctgatccaggtcaccaaagccttgtgagatcagactgcagagccagtctgtctctgagtcagtaaatgtagaatttggatttctcacaagttcctggcggtgtcttttttttttttaatattttttattaggtattttcctcatttacatttccaatgctatcccaaaagtcccccataccctcccccacactcccctacccacccactcccactttttggccctggtgttcccctgtactggggcatataaagtttgcaagtcctggtggtgtctttatgctgatctctagcccacactttgtgaggcactgggctatcccagtgtgctctcctcttccacagataccaaaagcacctgggtttgatgctcagacttctgagcacgttcttgttcaatctcttgcgtaagatttcctctcagatgagttgagtcagattctcatgtttaacagtgttttaggggattcacagaagcccaaactatcagttttcatttctgaaaaggctggaaaattttatgaaaaactttcaaaggtcagacagagccattttgagtcttttatgtgaccaagtatgaaccagatctttcctatctatggtctcccctttccaaaatatatcttttgtggggacacggcaaggaggaaagttaaatagaatctcaagctactaattttagaaaagaaaaaaatattaaactcttactaaagagctgtgggtagtggtacacacctgtaagcccagctctcaggagactgaggcaggaggattgcagtgagtcagagatcagcttcacctacaagcaaaaccctgccttacccctcaacctttccataaaaacagtcttacttgtgtaaaattaatttttaatacatatttgtgcacgatgtgtgtgcctggtgcccagggaagccaaaaaaatatgttcaatgtccctggaataggtggttatgagctgccatggaggtgatgggatttaaacccctgttctctgaaaaagcagctagttctcttaaccactgagccatctctacagccccattaaattgaattttattgtcattactcaatatgggagatggggtaatgataacaattttttttttataatactaagatgtttagctattttactctccttcacaagtgtagagtagaattttctagaagctacatgcatgatattatcgctctggtgttaacacataatggatttatcttgttaataaaagaattagtaaataatattttaaattttttctttgttttagttttaagatgattaatatctatagatactagtgtacattaagaaagcctttgggatcctcaatcattttgcatggttttagtaattttttaataacataaagaaggtctgacagattatgctaaagagctattgtggtatggattagaaatggcccccacaggctcctgtgttcaaacatcagctagcagtgctgctttgggttttggaacctttaagaggtggggccttgcttaaggagataggtcactggaggtgaaccagacttgcttccagtctccctctctgctgtcccaggaagccatcatatgaggagtttcacaacatacttctgctgccacagagtccctccagtaaggcacgtactggccactgtgtagtgtaccctctaaaactgagctagagctcccctctcctccctacactatctctgttgatgctttgtcacggtgatgaagaacataagtaaggcaaagacaagacatagtttggagactcacgtgagcatctcagccagactcaggcacagctgcgatgtgggaattatcaagcatgagatgcaaagcaatggaaaatgaatcgttatgacagaagcctacatctagtcttcccttcttcccattagtaataatagcccgtgttttagaagaacacatctttttggtgttctaggtagcttatattgcaaatgtggcacaatctaagagaaatctgggatgagggaacctcagtgaaagattctccttgatcagattgtcccactgatgattgatatgggaaggcccaacccactgtgaaaggcaccacccattggcaggatgactggggttatagaagcaaacaggctgagcatgagccagtgagcaagccagtaagcagtggcttctctttggaagaagcatggctttcttccgtggcttctgtttgatctctctcaatgatagattgtgacctagaagtataagctaaaataaaccatttcttacccata

SEQ ID NO: 25 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 26 - protein, 63 aa, Pan troglodytes
ELCDDDPPEITHATFKAMAYKEGTMLNCECKRGFRRIKSGSLYMLCTGNSSHSSWDNQCQCTS

SEQ ID NO: 27 - protein, 64 aa, Pan troglodytes
GHCREPPPWENEATERIYHFVVGQTVYYQCVQGYRALHRGPAESVCKMTHGKTRWTQPQLICTG

SEQ ID NO: 28 - protein, 63 aa, Macaca mulatta
ELCDDDPPKITHATFKAVAYKEGTMLNCECKRGFRRIKSGSPYMLCTGNSSHSSWDNQCQCTS

SEQ ID NO: 29 - protein, 64 aa, Macaca mulatta
GHCREPPPWENEATERIYHFVVGQMVYYQCVQGYRALHRGPAESICKMTHGKTRWTQPQLICTG

SEQ ID NO: 30 - protein, 58 aa, Mus musculus
ELCLYDPPEVPNATFKALSYKNGTILNCECKRGFRRLKELVYMRCLGNSWSSNCQCTS

SEQ ID NO: 31 - protein, 64 aa, Mus musculus
GHCREPPPWKHEDSKRIYHFVEGQSVHYECIPGYKALQRGPAISICKMKCGKTGWTQPQLTCVD

SEQ ID NO: 32 - protein, 19 aa, Pan troglodytes
VAVAGCVFLLISVLLLSGL

SEQ ID NO: 33 - protein, 19 aa, Macaca mulatta
VAVAGCVFLLISVLLLSGL

SEQ ID NO: 34 - protein, 21 aa, Mus musculus
VAVASCLFLLISILLLSGLTW

SEQ ID NO: 35 - protein, 6 aa, Pan troglodytes
TWQRRQ

SEQ ID NO: 36 - protein, 13 aa, Macaca mulatta
TWQRRQRKNRRTI

SEQ ID NO: 37 - protein, 11 aa, Mus musculus
QHRWRKSRRTI

SEQ ID NO: 38 - protein, 15 aa, synthetic construct
  REGION 1..15: note = synthetic linker sequence
SGGGGSGGGGSGGGG

SEQ ID NO: 39 - protein, 19 aa, synthetic construct
  REGION 1..19: note = synthetic linker sequence
ASTKGPSVFPLAPSSSGSG

SEQ ID NO: 40 - protein, 15 aa, synthetic construct
  REGION 1..15: note = synthetic linker sequence
GGGGSGGGGSGGGGS

SEQ ID NO: 41 - protein, 18 aa, synthetic construct
  REGION 1..18: note = synthetic whitlow linker sequence
GSTSGSGKPGSGEGSTKG

SEQ ID NO: 42 - DNA, 54 bp, synthetic construct
  misc_feature 1..54: note = synthetic whitlow linker sequence
  source 1..54: mol_type = other DNA
ggcagcaccagcggcagcggcaaaccgggcagcggcgaaggcagcaccaaaggc

SEQ ID NO: 43 - protein, 25 aa, synthetic construct
  REGION 1..25: note = synthetic linker sequence
GGGGSGGGGSGGGGSGGGGSGGGGS

SEQ ID NO: 44 - DNA, 75 bp, synthetic construct
  misc_feature 1..75: note = synthetic linker sequence
  source 1..75: mol_type = other DNA
ggcggtggtggttctggaggcggtggcagcggtggaggtggctcaggaggaggaggtagcggcggcggagggagt

SEQ ID NO: 45 - protein, 233 aa, Homo sapiens
AEPKSPDKTHTCPPCPAPPVAGPSVFLFPPKPKDTLMIARTPEVTCWVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 46 - protein, 46 aa, Homo sapiens
TTTPAPRPPTPAPTIASQPLSLRPEACRPAAGGAVHTRGLDFACDI

SEQ ID NO: 47 - protein, 20 aa, Homo sapiens
AEPKSPDKTHTCPPCPKDPK

SEQ ID NO: 48 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 49 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 50 - protein, 23 aa, Homo sapiens
LGLLVAGVLVLLVSLGVAIHLCC

SEQ ID NO: 51 - protein, 26 aa, Homo sapiens
VAAILGLGLVLGLLGPLAILLALYLL

SEQ ID NO: 52 - protein, 25 aa, Homo sapiens
ALIVLGGVAGLLLFIGLGIFFCVRC

SEQ ID NO: 53 - protein, 23 aa, Homo sapiens
LCYLLDGILFIYGVILTALFLRV

SEQ ID NO: 54 - protein, 26 aa, Homo sapiens
WVLVVVGGVLACYSLLVTVAFIIFWV

SEQ ID NO: 55 - protein, 24 aa, Homo sapiens
IYIWAPLAGTCGVLLLSLVITLYC

SEQ ID NO: 56 - protein, 24 aa, Homo sapiens
ALPAALAVISFLLGLGLGVACVLA

SEQ ID NO: 57 - protein, 220 aa, Homo sapiens; identical to SEQ ID NO: 48

SEQ ID NO: 58 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 59 - protein, 164 aa, Homo sapiens; identical to SEQ ID NO: 49

SEQ ID NO: 60 - protein, 113 aa, Homo sapiens
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - DNA, 339 bp, Homo sapiens
  source 1..339: mol_type = genomic DNA
ctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 62 - protein, 233 aa, Homo sapiens; identical to SEQ ID NO: 45

SEQ ID NO: 63 - protein, 46 aa, Homo sapiens; identical to SEQ ID NO: 46

SEQ ID NO: 64 - protein, 20 aa, Homo sapiens
AEPKSPDKTHTCPPCPKDPK

SEQ ID NO: 65 - DNA, 1316 bp, synthetic construct
  misc_feature 1..1316: note = synthetic mbIL2 sequence
  source 1..1316: mol_type = other DNA
atgctcctgctcgtgacttcacttcttctctgtgaactcccacaccccgcgtttttgcttatccctcatcatcaccatcaccacgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttctggaggaggtagtggaggcggcgggagcggtgggggagggtctgggggtgggggatccggaggtgggtcactgcaggagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctg

SEQ ID NO: 66 - protein, 41 aa, synthetic construct
  REGION 1..41: note = synthetic T2A and purin cleavage site sequence
RKRRGKPIPNPLLGLDSTSGSGEGRGSLLTCGDVEENPGPG

SEQ ID NO: 67 - DNA, 123 bp, synthetic construct
  misc_feature 1..123: note = synthetic T2A and purin cleavage site sequence
  source 1..123: mol_type = other DNA
cggaagagaagaggcaagcccatccccaacccactgctgggcctggatagcacctccggctcaggagagggcagaggctctctgctgacctgcggcgacgtggaagagaacccgggcccgggc

SEQ ID NO: 68 - protein, 499 aa, synthetic construct
  REGION 1..499: note = Synthetic FMC63-41BB-CD3z CAR sequence
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - DNA, 1497 bp, synthetic construct
  misc_feature 1..1497: note = synthetic FMC63-41BB-CD3z CAR sequence
  source 1..1497: mol_type = other DNA
atgcttctcctggtgacaagccttctgctctgtgagttaccacacccagcattcctcctgattcctgaacagaagctgataagtgaggaggacttggacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 70 - protein, 22 aa, synthetic construct
  REGION 1..22: note = Synthetic signal peptide sequence
MLLLVTSLLLCELPHPAFLLIP

SEQ ID NO: 71 - DNA, 66 bp, synthetic construct
  misc_feature 1..66: note = Synthetic signal peptide sequence
  source 1..66: mol_type = other DNA
atgcttctcctggtgacaagccttctgctctgtgagttaccacacccagcattcctcctgattcct

SEQ ID NO: 72 - protein, 10 aa, synthetic construct
  REGION 1..10: note = synthetic Myc tag sequence
EQKLISEEDL

SEQ ID NO: 73 - DNA, 30 bp, synthetic construct
  misc_feature 1..30: note = synthetic Myc tag sequence
  source 1..30: mol_type = other DNA
gaacagaagctgataagtgaggaggacttg

SEQ ID NO: 74 - protein, 242 aa, synthetic construct
  REGION 1..242: note = synthetic FMC63 scFv sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 75 - DNA, 726 bp, synthetic construct
  misc_feature 1..726: note = synthetic FMC63 scFv sequence
  source 1..726: mol_type = other DNA
gacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagc

SEQ ID NO: 76 - protein, 71 aa, synthetic construct
  REGION 1..71: note = synthetic hinge and transmembrane domain sequence
GSTTTPAPRPPTPAPTIASQPLSLRPEACRPAAGGAVHTRGLDFACDIYIWAPLAGTCGVLLLSLVITLYC

SEQ ID NO: 77 - DNA, 213 bp, synthetic construct
  misc_feature 1..213: note = synthetic hinge and transmembrane domain sequence
  source 1..213: mol_type = other DNA
ggatccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 78 - protein, 41 aa, synthetic construct
  REGION 1..41: note = synthetic costimulatory domain sequence
KRGRKKLLYIFKQPFMRPVQTTQEEDGCSCRFPEEEEGGCE

SEQ ID NO: 79 - DNA, 123 bp, synthetic construct
  misc_feature 1..123: note = synthetic costimulatory domain sequence
  source 1..123: mol_type = other DNA
aaacggggcagaaagaaactcctgtatatattcaaacaaccatttatgagaccagtacaaactactcaagaggaagatggctgtagctgccgatttccagaagaagaagaaggaggatgtgaa

SEQ ID NO: 80 - protein, 113 aa, synthetic construct; identical to SEQ ID NO: 60
  REGION 1..113: note = synthetic CAR CD3z domain sequence

SEQ ID NO: 81 - DNA, 339 bp, synthetic construct; identical to SEQ ID NO: 61
  misc_feature 1..339: note = synthetic CAR CD3z domain sequence
  source 1..339: mol_type = other DNA